(19) United States
(12) Patent Application Publication
(10) Pub. No.: US 2014/0017268 A1
(43) Pub. Date: Jan. 16, 2014

(54) Composition and Methods for Detecting or Preventing Lawsonia intracellularis Infections

(71) Applicant: REGENTS OF THE UNIVERSITY OF MINNESOTA, St. Paul, MN (US)

(72) Inventors: Connie Gebhart, St. Paul, MN (US); Fabio Vannucci, St. Paul, MN (US)

(73) Assignee: REGENTS OF THE UNIVERSITY OF MINNESOTA, St. Paul, MN (US)

(21) Appl. No.: 13/910,861

(22) Filed: Jun. 5, 2013

Related U.S. Application Data
(60) Provisional application No. 61/655,793, filed on Jun. 5, 2012.
(60) Provisional application No. 61/792,648, filed on Mar. 15, 2013.

(51) Int. Cl. C07K 14/195; G01N 33/53

(52) U.S. Cl. CPC C07K 14/195; G01N 33/5308
USPC 424/185.1; 435/7.1; 530/300; 530/387.9

(57) ABSTRACT

The invention relates to vaccines for preventing Lawsonia intracellularis infections and tests for the detection of the presence of L. intracellularis antibodies in biological samples. The present invention also relates to nucleic acid sequences encoding novel L. intracellularis proteins.

### PRIORITY OF INVENTION

This application claims priority from U.S. Provisional Application No. 61/655,793, filed Jun. 5, 2012, and U.S. Provisional Application No. 61/792,648, filed on Mar. 15, 2013. The entire contents of these provisional applications are hereby incorporated herein by reference.

### BACKGROUND OF THE INVENTION

Lawsonia intracellularis is the causative agent of proliferative enteropathy (PE). This disease affects various animal species, including nonhuman primates, and is endemic in pigs and an emerging concern in horses. Non-pathogenic variants obtained through multiple passages in cell culture do not induce disease, but bacterial isolates at low-passage induce clinical and pathological changes typical of PE.

L. intracellularis is a fastidious intracellular bacterium that causes an intestinal hyperplasic disease characterized by thickening of the mucosa of the intestine due to enterocyte proliferation. Cell proliferation is directly associated with bacterial infection and replication in the intestinal epithelium. As a result, mild to severe diarrhea is the major clinical sign described in infected animals. Since the 1990s, PE has been endemic in swine herds and has been occasionally reported in various other species, including nonhuman primates, wild mammalians and ratite birds. Outbreaks among foals began to be reported on breeding farms worldwide within the last decade. Therefore, PE is now considered an emerging disease in horses.

Although porcine PE was first reported in 1931, the causative bacterium was isolated only in 1993 using rat small intestinal cells (IEC-18) in strict microaerophilic environmental conditions. Since then, various cell lines have supported L. intracellularis growth in vitro, including insect and avian cells lines. To date, bacterial growth in axenic (cell-free media) has not been reported. Regardless of the cell type, the dynamics of the infection in vitro requires actively dividing cells in a microaerophilic atmosphere with the peak of the infection at six to seven days post-inoculation. McOrist et al (1995) chronologically described the dynamics of the infection and bacterial replication in porcine intestinal epithelial cells (IPEC-J2). Most events closely resembled events observed at cellular level in infected animals, including multiplication of the bacteria free in the cell cytoplasm. The dynamics of the infection has been well-characterized, but, so far, little is known about the genetic basis for the virulence, pathogenesis or physiology of L. intracellularis (Jacobson M, Fellstrom C, Jensen-Waern M (2010) Porcine proliferative enteropathy: an important disease with questions remaining to be solved. Vet J 184: 264-268).

Spontaneously attenuated isolates obtained through multiple passages in cell culture have not been successful at inducing typical PE lesions or attenuating its virulence in experimentally-infected pigs. Conversely, bacterial isolates at low-passage induce clinical and pathological changes typical of PE (Guedes R M, Gebhart C J (2003) Onset and duration of fecal shedding, cell-mediated and humoral immune responses in pigs after challenge with a pathogenic isolate or attenuated vaccine strain of Lawsonia intracellularis. Vet Microbiol 91: 135-145). Various standard DNA-based typing techniques, such as pulsed field gel electrophoresis (PFGE), multilocus sequence typing (MLST) and variable number tandem repeat (VNTR) have shown identical genotypes in both pathogenic (low-passage) and non-pathogenic (high-passage) variants (Oliveira A R, Gebhart C J. Differentiation of Lawsonia intracellularis isolates from different animal species using Pulsed field gel electrophoresis (PFGE); 2008; Saint Paul; Kelley M F, Gebhart C J, Oliveira S. Multilocus sequencing typing system for Lawsonia intracellularis; 2010; Chicago, Ill. CRWAD; Beckler D C, Kapur V, Gebhart C J. Molecular epidemiologic typing of Lawsonia intracellularis. In: CRWAD, editor; 2004; Chicago, Ill. pp. 124). As a result, it is believed that their phenotypic properties occur at the transcriptional level.

The current understanding of L. intracellularis infection, treatment, control and detection of the disease has been hampered by the fact that L. intracellularis cannot be cultivated in cell-free media. Thus, there is an ongoing need for methods of detecting and preventing L. intracellularis infections.

### SUMMARY OF THE INVENTION

It was surprisingly found that pathogenic L. intracellularis produces at a high expression level certain genes encoding proteins to which an infected animal may generate antibodies. The detection of these specific antibodies can be diagnostic for the disease. Further, these high expression level proteins can be used to induce protective immunity against L. intracellularis.

In certain embodiments, the present invention provides a method for detecting the presence of a L. intracellularis biomarker in an animal, comprising identifying in a physiological sample from the animal an antibody specific for an amino acid of SEQ ID NO:2 (LI0447), LI0461, LI0267, LI0809, LI1158, LI1159 or LIC060 (Table 13).

LI0447 Amino Acid SEQ ID NO: 2
(NCBI Reference Sequence: YP_594823.1)
MGDCHSSRNI VTYILKQPTQ EHWPGQPVPL FGLASNGACL
ARYITITTGR LLLHRFTLTT IINNGGLLSV ALAEDHSSWV
LPSVLPYEAR TFLSINP

In certain embodiments, the LI0447 is encoded by a nucleic acid of SEQ ID NO:1 (NCBI Reference Sequence: NC_{—}008011.1):

atgggcgact gtcattcatc taggaatata gttacctata
ttctcaagca acctacccag gaacattggc cgggtcagcc
tgttccccta tttggtcttg cttcaaacgg ggcttgccta
gctcgctata tcactataac gactggtaga ctcttactcc
accgtttcac ccttaccact attattaata atggcggttt
actttctgtt gcgcttgccg aagatcactc ttcctgggtg
ttacccagcg ttttacccta tgaagcccgg actttcctct
ccataaatcc ataa

As used herein, the phrase “physiological sample” is meant to refer to a biological sample obtained from a mammal that contains antibodies. For example, a physiological sample can be a sample collected from an individual pig or horse, such as fluid sample (e.g., blood). The term “biomarker” is generally defined herein as a biological indicator, such as a particular molecular feature, that may affect or be related to diagnosing or predicting an individual's health. For example, in certain embodiments of the present invention, the biomarker comprises a LI0447 gene.

As used herein, an antibody that is “specific for LI0447” means that the antibody binds with high affinity to LI0447, but does not significantly bind to other proteins from L. intracellularis.

In certain embodiments, the present invention provides a method for detecting the presence of L. intracellularis in an animal, comprising identifying in an amino acid sample from the animal an amino acid of SEQ ID NO:2, LI0461, LI0267, LI0809, LI1158, LI1159 or LIC060 wherein the presence of LI0447, LI0461, LI0267, LI0809, LI1158, LI1159 or LIC060 is indicative of a pathogenic L. intracellularis infection in the animal. In certain embodiments, the animal is a pig or a horse.

In certain embodiments, the present invention provides a method for diagnosing a pathogenic L. intracellularis infection in an animal comprising detecting the presence of a biomarker in a physiological sample from the animal, wherein the presence of the biomarker is indicative of the disease. In certain embodiments, the sample comprises an antibody. In certain embodiments, the biomarker is an amino acid of SEQ ID NO:2, LI0461, LI0267, LI0809, LI1158, LI1159 or LIC060.

In certain embodiments, the present invention provides an immunogenic composition comprising a polypeptide encoded by a nucleic acid sequence having at least 90% identity to a nucleic acid sequence SEQ ID NO:1, or a nucleic acid encoding LI0461, LI0267, LI0809, LI1158, LI1159 or LIC060.

In certain embodiments, the present invention provides an immunogenic composition comprising a polypeptide encoded by a nucleic acid sequence consisting of nucleic acid sequence SEQ ID NO:1 or a nucleic acid encoding LI0461, LI0267, LI0809, LI1158, LI1159 or LIC060.

In certain embodiments, the immunogenic composition further comprises a physiologically-acceptable vehicle. In certain embodiments, the immunogenic composition further comprises an effective amount of an adjuvant. In certain embodiments, the polypeptide is capable of generating an antibody that specifically recognizes the polypeptide, and wherein the amount of the immunogenic composition is effective to ameliorate colonization or infection by L. intracellularis in a susceptible mammal.

In certain embodiments, the present invention provides a method of treating a susceptible animal against colonization or infection by L. intracellularis comprising administering to the animal an effective amount of the immunogenic composition described above, wherein the polypeptide is capable of generating an antibody specific to the polypeptide, and wherein the amount of the immunogenic composition is effective to ameliorate colonization or infection by L. intracellularis in the susceptible animal. In certain embodiments, the immunogenic composition is administered by subcutaneous injection, by intramuscular injection, by oral ingestion, intranasally, or combinations thereof. In certain embodiments, the animal is a pig or a horse.

In certain embodiments, the present invention provides a vaccine comprising an immunogenic amount of the composition described above, which amount is effective to inhibit in an animal an infection by L. intracellularis, in combination with a physiologically-acceptable, non-toxic vehicle.

In certain embodiments, the present invention provides an isolated L. intracellularis protein having the amino acid sequence of SEQ ID NO:2, LI0461, LI0267, LI0809, LI1158, LI1159 or LIC060.

In certain embodiments, the present invention provides a purified antibody that binds specifically to the amino acid of SEQ ID NO:2, LI0461, LI0267, LI0809, LI1158, LI1159 or LIC060.

### BRIEF DESCRIPTION OF THE FIGURES AND TABLES

FIG. 1. Schematic representation of the L. intracellularis genome. Distribution of genes expressed by the pathogenic (black circles) and the non-pathogenic (gray circles) variants. Overlapping zones represent genes expressed in both variants.

FIG. 2. Functional categories of genes expressed by the pathogenic and non-pathogenic homologous L. intracellularis isolate of PHE/MN1-00. Black and gray bars represent the number of genes expressed by the pathogenic and non-pathogenic variants, respectively.

FIG. 3. Average log-transformed RPKM (log₂ [RPKM]) of the 319 genes commonly expressed by the pathogenic (y-axis) and non-pathogenic (x-axis) variant. The trend line represent a linear regression model (p-value <0.05; r²=0.809).

FIG. 4. Correlation between RNA-seq and qRT-PCR. Plot demonstrating the relative quantification of 10 unlinked genes by qRT-PCR (y-axis) and the transcript levels generated by RNA-seq (x-axis). Genes commonly expressed by the pathogenic (♦) and non-pathogenic (♦) variants. Genes exclusively expressed by the pathogenic (⋄) and non-pathogenic (⋄) variants.

FIG. 5. Reproducibility of biological replicates. RPKM of replicate 1 plotted on the y-axis and the replicate 2 on the x-axis. Each spot represents a single gene. Black circles represent genes expressed by the pathogenic isolate (PHE/MN1-00 at passage 10) and the linear regression (solid trendline−r2=0.862). Gray squares represent genes expressed by the non-pathogenic isolate (PHE/MN1-00 at passage 60) and the linear regression (dashed trendline−r2=0.813).

FIGS. 6A-6B. RPKM representing the transcription levels (y-axis) and the number of mapped genes (x-axis) onto the L. intracellularis reference genome. (A) Pathogenic variant showing 720 expressed genes. (B) Non-pathogenic variant showing 330 expressed genes. The locus tags of the four highest expressed genes are described.

FIGS. 7A-7D. Laser capture microdissection of an intestinal crypt infected with L. intracellularis (A-B) and evaluation of RNA quality from microdissected cells (C-D). (A) Hematoxylin stained cryosection of infected ileal mucosa. (B) Microdissected intestinal crypt captured in the thermoplastic film of the LCM cap prior to RNA isolation. (C) Agilent Bioanalyzer data showing bacterial and eukaryotic ribosomal RNA in infected and non-infected cells. (D) One-step RT-PCR products of three protein-encoding genes of L. intracellularis.

FIGS. 8A-8B. Correlation between RNA-seq and qRT-PCR expression data. (A) Plot of the relative fold-change in gene expression of 16 porcine genes from infected enterocytes by RNA-seq (x-axis) and qRT-PCR (y-axis). R²=0.92 (p<0.05). (B) Plot of the relative quantification of ten L. intracellularis genes by RNA-seq (x-axis) and the transcript levels generated by qRT-PCR (y-axis). R²=0.81 (p<0.05).

FIGS. 9A-9B. Cellular network and canonical pathway analysis of genes differentially expressed by enterocytes infected with L. intracellularis. (A) Molecular interaction representing part of the protein biosynthesis network which was most correlated with the set of genes significantly up-regulated in infected cells. Strong interaction between the main canonical pathway (CP: EIF2 signaling) identified in the differentially expressed genes and the protein biosynthesis network. (B) Canonical pathways (x-axis) most associated with genes differentially expressed based on the −log of p-value calculated by Fisher's exact test (y-axis).

FIGS. 10A-10B. Proposed model for host-pathogen interaction in porcine enterocytes infected with L. intracellularis. (A) Infected enterocyte. Apical membrane exhibiting down-regulation of genes involved in nutrient acquisition and electrolyte secretion (CUBN, SLC10A2, SLC5A1, SLC7A3, IS, PPAP2A and CLCA1) and up-regulation of copper uptake protein (SLC31A1, SLC2A1 and MHC-1). Basolateral membrane exhibiting up-regulation of glucose transporter (SLC2A1) and MHC class I genes. (B) Intracellular bacterium. High expression (rubY, rubA, PcrH, SseC and Sse?) of genes included in the oxidative stress protection system: redox enzymes, Cu—Zn superoxide dismutase (sodC) and rubrerythrin-rubredoxin (rubY-rubA) operon (Adapted from Lumppio et al. 2001). Moderate expression genes encoding the basal body of the type III secretion system (TIIISS) and high expression of TIIISS effector proteins (PcrH-SseC-Sse? operon). Genetic organization and gene expression (log_{2 [RPKM]}) of the rubY-rubA and PcrH-SseC-Sse operons.

Table 1. Chromosomal genes showing highest transcript levels exclusively expressed by the pathogenic variant of the L. intracellularis isolate PHE/MN1-00 according to the putative biological function.

Table 2. Genes exclusively expressed by the non-pathogenic variant of the L. intracellularis isolate PHE/MN1-00.

Table 3. Plasmid-encoded genes with highest transcript levels exclusively expressed by the pathogenic variant of the L. intracellularis isolate PHE/MN1-00.

Table 4. Comparison between the transcription levels of genes commonly expressed by the pathogenic and non-pathogenic homologous L. intracellularis isolate PHE/MN1-00.

Table 5. Primers used for validation of RNA-seq expression data by quantitative reverse transcriptase PCR assay.

Table 6. Top 20 highly expressed genes by L. intracellularis in the enterocyte cytoplasm.

Table 7. Four cell networks most associated with the genes differentially expressed in Lawsonia-infected enterocytes.

Table 8. Functional clustering associated with genes differentially expressed in Lawsonia-infected enterocytes.

Table 6. The top 20 highly expressed genes by L. intracellularis in the enterocyte cytoplasm.

Table 7. The four cell networks most associated with the genes differentially expressed in Lawsonia-infected enterocytes.

Table 8. Functional clustering associated with genes differentially expressed in Lawsonia-infected enterocytes.

Table 9. Primers used for one-step RT-PCR and for validation of RNA-seq expression data by quantitative reverse transcriptase PCR assay.

Table 10. Sequencing and mapping of each biological replicate.

Table 11. Genes differentially expressed by enterocytes infected with L. intracellularis.

Table 12. Predictive analysis of the highest expressed L. intracellularis genes encoding hypothetical proteins.

Table 13. Highly expressed Lawsonia gene transcripts.

### DETAILED DESCRIPTION OF THE INVENTION

### Detection Assays

The present invention provides methods for detecting antibodies specific for LI0447 (“LI0447 antibodies”) in a sample or in vivo. In certain embodiments, Western blots may be used to determine the presence and/or quantity of LI0447 antibodies in the sample. In certain embodiments or a competitive assay (for example, radioimmunoassay) may be used to determine the presence and/or quantity of LI0447 antibodies in the sample. For example, one can contact a sample suspected of containing LI0447 antibodies with a binding ligand (i.e., LI0447 protein) and detecting the presence or the quantity of bound LI0447 protein/LI0447 antibody complex. In certain embodiments, the complex is detected by means of nuclear magnetic resonance, fluorescent capillary electrophoresis, lateral flow devices, colorimetry, chemiluminescence, fluorescence, western blots, microarrays, enzyme linked immunosorbent assay (ELISA), radioHPLC, single photon emission computed tomography (SPECT), or positron emission tomography (PET), radioimmunoassay (RIA), immunoradiometric assay, fluoroimmunoassay, chemiluminescent assay, and bioluminescent assay, though several others are well known to those of ordinary skill. The steps of various useful immunodetection methods have been described in the scientific literature.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

Further methods include the detection of primary immune complexes by a two-step approach. A second binding ligand such as an antibody that has binding affinity for the LI0447 protein/LI0447 antibody complex is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different ligands. A first step biotinylated, LI0447 antigen is used to detect the target LI0447 antibodies, and a second step antibody is then used to detect the biotin attached to the complexed binding ligand. In this method the sample to be tested is first incubated in a solution containing the first step antigen. If the target antibody is present, some of the antigen binds to the antibody to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

The diagnostic assay format that may be used in the present invention could take any conventional format such as ELISA or other platforms such as luminex or biosensors. The present invention detects the presence of a LI0447 protein/LI0447 antibody complex. This sequence can readily be modified to facilitate diagnostic assays, for example a tag (such as GFP) can be added to the targeting antigen to increase sensitivity. In one exemplary ELISA, LI0447 antigen is immobilized onto a selected surface exhibiting protein affinity, such as a well in a polystyrene microtiter plate. Then, a test composition suspected of containing the LI0447 antibodies, such as a clinical sample (e.g., a biological sample obtained from the subject), is added to the wells. After binding and/or washing to remove non-specifically bound immune complexes, the bound antibodies may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antibodies are immobilized onto the well surface and/or then contacted with binding agents (i.e., LI0447 antigen). After binding and/or washing to remove non-specifically bound immune complexes, the bound anti-binding agents are detected. Where the initial binding agents are linked to a detectable label, the immune complexes may be detected directly. Again, the immune complexes may be detected using a second antibody that has binding affinity for the first binding agents, with the second antibody being linked to a detectable label.

Another ELISA in which the antibodies are immobilized, involves the use of antigen competition in the detection. In this ELISA, labeled antigens are added to the wells, allowed to bind, and/or detected by means of their label. The amount of an antibody in an unknown sample is then determined by mixing the sample with the labeled antigens during incubation with coated wells. The presence of an antibody in the sample acts to reduce the amount of antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Other detection assays can be used such as lateral flow devices and magnetic bead-based methods. These methods are generally known in the art.

Irrespective of the format employed, these detection assays have certain features in common, such as coating, incubating and binding, washing to remove non-specifically bound species, and detecting the bound immune complexes.

In coating a plate with LI0447 antigens, one will generally incubate the wells of the plate with a solution of the antigen, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of the antigen to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions include diluting the immune complex with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

A “control” sample is a cell, tissue, sample, or subject of the same type as a test sample. The control may, for example, be examined at precisely or nearly the same time the test sample is examined. The control may also, for example, be examined at a time distant from the time at which the test sample is examined, and the results of the examination of the control may be recorded so that the recorded results may be compared with results obtained by examination of a test sample. The control may also be obtained from another source or similar source other than the test sample, where the test sample is obtained from an animal suspected of having L. intracellularis.

A “test” sample is one being examined.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or is an atom or a molecule that permits the specific detection of a compound comprising the marker in the presence of similar compounds without a marker. Detectable markers include, but are not limited to, radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

The detectable labels used in the assays of the present invention to diagnose L. intracellularis, these labels are attached to the LI0447 antigen, can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue Published by Molecular Probes, Inc., Eugene, Oreg. Patents that described the use of such labels include U.S. Pat. Nos. 3,817,837; 3,850,752; 3,939,350; 3,996,345; 4,277,437; 4,275,149; and 4,366,241.

Primary and secondary labels can include undetected elements as well as detected elements. Useful primary and secondary labels in the present invention can include spectral labels such as green fluorescent protein, fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes.™., and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g. polystyrene, polypropylene, latex, etc.) beads. The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Exemplary labels that can be used include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate (kits available from Life Technologies/Gibco BRL, and Boehringer-Mannheim)); 3) fluorescence using, e.g., an enzyme such as alkaline phosphatase, together with the substrate AttoPhos (Amersham) or other substrates that produce fluorescent products, 4) fluorescence (e.g., using Cy-5 (Amersham), fluorescein, and other fluorescent tags); 5) radioactivity. Other methods for labeling and detection will be readily apparent to one skilled in the art.

Where the LI0447 antibodies are contemplated to be detected in a clinical setting, the labels are preferably non-radioactive and readily detected without the necessity of sophisticated instrumentation. In certain embodiments, detection of the labels will yield a visible signal that is immediately discernible upon visual inspection. One example of detectable secondary labeling strategies uses an antigen linked to an enzyme (typically by recombinant or covalent chemical bonding). The antibody/antigen complex is detected when the enzyme reacts with its substrate, producing a detectable product. In certain embodiments, enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a fluorescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer, and 3,3,5,5′-tetramethylbenzidine (TMB), 3,3′ diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures known to those skilled in the art and kits for performing enzyme immunoassays are available as described above.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

### Nucleic Acids Encoding Proteins

It is well-known in the art that different nucleic acid sequences can encode one and the same protein due to the degeneracy of the genetic code. This phenomenon can result in a heterology of about 30% for two nucleic acid sequences still encoding the same protein. One embodiment of the present invention relates to nucleic acid sequences encoding a L. intracellularis protein and to parts of that nucleic acid sequence that encode an immunogenic fragment of that protein, wherein those nucleic acid sequences or parts thereof have a level of homology with the nucleic acid sequence of SEQ ID NO: 1 of at least 70%.

LI0447 Nucleic Acid SEQ ID NO: 1
(NCBI Reference Sequence: NC_008011.1)
atgggcgact gtcattcatc taggaatata gttacctata
ttctcaagca acctacccag gaacattggc cgggtcagcc
tgttccccta tttggtcttg cttcaaacgg ggcttgccta
gctcgctata tcactataac gactggtaga ctcttactcc
accgtttcac ccttaccact attattaata atggcggttt
actttctgtt gcgcttgccg aagatcactc ttcctgggtg
ttacccagcg ttttacccta tgaagcccgg actttcctct
ccataaatcc ataa

The nucleic acid sequence encoding this L. intracellularis protein or the part of the nucleic acid sequence has at least 80%, 90%, or even 95% homology with the nucleic acid sequence of SEQ ID NO: 1. In certain embodiments, the homology level is at 98% or even 100%.

In certain embodiments, the nucleic acid encodes the protein of SEQ ID NO:2, or an immunogenic fragment of this polypeptide:

LI0447 Amino Acid SEQ ID NO: 2
(NCBI Reference Sequence: YP_594823.1)
MGDCHSSRNI VTYILKQPTQ EHWPGQPVPL FGLASNGACL
ARYITITTGR LLLHRFTLTT IINNGGLLSV ALAEDHSSWV
LPSVLPYEAR TFLSINP

In certain embodiment of the invention, DNA fragments comprising a nucleic acid can be engineered into a vector, such as a plasmid. Such vectors are useful for enhancing the amount of DNA for various uses, such as an immunogenic antigen or vaccine.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res.,19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The terms “protein,” “peptide” and “polypeptide” are used interchangeably herein.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

As discussed above, the terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule that is complementary or hybridizes to a sequence in a gene of interest and remains stably bound under stringent conditions (as defined by methods well known in the art). Thus, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Synthetic” polynucleotides are those prepared by chemical synthesis. As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, LI0477, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ 81.5° C.+16.6 (log M)+0.41 (% GC)−0.61 (% form)−500/L

where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. Guidance as to appropriate amino acid substitutions that do not affect biological activity of the protein of interest are well known in the art. Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally-occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations.”

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

A “host cell” is a cell which has been transformed, or is capable of transformation, by an exogenous nucleic acid molecule. Thus, “transformed,” “transgenic,” and “recombinant” refer to a host cell or organism into which a heterologous nucleic acid molecule has been introduced. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically includes sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will have the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source.

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of single-stranded mutagenesis. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Genome” refers to the complete genetic material of an organism.

“Coding sequence” refers to a DNA or RNA sequence that codes for a specific amino acid sequence and excludes the non-coding sequences. For example, a DNA “coding sequence” or a “sequence encoding” a particular polypeptide, is a DNA sequence which is transcribed and translated into a polypeptide in vitro or in vivo when placed under the control of appropriate regulatory elements. The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. It may constitute an “uninterrupted coding sequence,” i.e., lacking an intron, such as in a cDNA or it may include one or more introns bounded by appropriate splice junctions. An “intron” is a sequence of RNA that is contained in the primary transcript but that is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The term “RNA transcript” refers to the product resulting from RNA polymerase catalyzed transcription of a DNA sequence. When the RNA transcript is a perfect complimentary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA—box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

A control element, such as a promoter, “directs the transcription” of a coding sequence in a cell when RNA polymerase binds the promoter and transcribes the coding sequence into mRNA, which is then translated into the polypeptide encoded by the coding sequence.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones having a population of daughter cells containing the exogenous DNA.

“Operably-linked” refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” or “translation stop codon” or “stop codon” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. The change of at least one nucleotide in a nucleic acid sequence can result in an interruption of the coding sequence of the gene, e.g., a premature stop codon.

Sources of nucleotide sequences from which the present nucleic acid molecules can be obtained include any prokaryotic or eukaryotic source. For example, they can be obtained from a mammalian cellular source. Alternatively, nucleic acid molecules of the present invention can be obtained from a library.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

Target L. intracellularis Proteins

One embodiment of the present invention provides an isolated and purified L. intracellularis protein of Table 13 (e.g., LI0447) that is at least 70% homologous to the amino acid sequence as depicted in Table 13 and to immunogenic fragments of this protein. In certain embodiments the L. intracellularis proteins that have a sequence homology of at least 80%, 90%, 95%, 98% or even 100% homology to the amino acid sequence as depicted in Table 13 and to immunogenic fragments of such proteins.

One of skill in the art would recognize that natural variations can exist between individual L. intracellularis strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities have been described previously. Such amino acid substitutions of the exemplary embodiments of this invention, as well as variations having deletions and/or insertions are within the scope of the invention as long as the resulting proteins retain their immune reactivity. L. intracellularis proteins according to the invention, when isolated from different field isolates, may have homology levels of about 70%, while still representing the same protein with the same immunological characteristics. Those variations in the amino acid sequence of a certain protein according to the invention that still provide a protein capable of inducing an immune response against infection with L. intracellularis or at least against the clinical manifestations of the infection are considered as “not essentially influencing the immunogenicity”.

One of skill in the art would also recognize that when a protein is used in certain embodiments (e.g. vaccination purposes or for raising antibodies), it is not necessary to use the whole protein, but instead an immunogenic fragment of the protein can be used. An “immunogenic fragment” is understood to be a fragment of the full-length protein that still has retained its capability to induce an immune response in the host, i.e. comprises a B- or T-cell epitope. A variety of techniques is available to easily identify DNA fragments encoding antigenic fragments (determinants). The method described by Geysen et al (Patent Application WO 84/03564, Patent Application WO 86/06487, U.S. Pat. No. 4,833,092, Proc. Natl. Acad. Sci. 81: 3998-4002 (1984), J. Imm. Meth. 102, 259-274 (1987)). Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and U.S. patent application NTIS U.S. Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzowsky for HIV-epitopes; The FASEB Journal 5:2412-2418 (1991).

### Complexes of Target Proteins and Other Molecules

In certain embodiments, the target protein can be conjugated or linked to a peptide or to a polysaccharide. For example, immunogenic proteins well-known in the art, also known as “carriers,” may be employed. Useful immunogenic proteins include keyhole limpet hemocyanin (KLH), bovine serum albumin (BSA), ovalbumin, human serum albumin, human gamma globulin, chicken immunoglobulin G and bovine gamma globulin. Useful immunogenic polysaccharides include group A Streptococci polysaccharide, C-polysaccharide from group B Streptococci, or the capsular polysaccharide of Streptococci pnuemoniae. Alternatively, polysaccharides of other pathogens that are used as vaccines can be conjugated or linked to the target protein as depicted in Table 13.

### Vaccines of the Invention

The present invention provides a vaccine for use to protect mammals against the colonization and/or infection of L. intracellularis bacteria. In one embodiment of this invention, a target protein as depicted in Table 13 (e.g., LI0447) can be delivered to a mammal in a pharmacologically acceptable vehicle. As one skilled in the art will appreciate, it is not necessary to use the entire target protein.

As one skilled in the art will also appreciate, it is not necessary to use target protein that is identical to native target protein. The modified target protein can correspond essentially to the corresponding native target protein. As used herein “correspond essentially to” refers to a target protein epitope that will elicit a immunological response at least substantially equivalent to the response generated by a native target protein. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest. Vaccines of the present invention can also include effective amounts of immunological adjuvants, known to enhance an immune response.

To immunize a subject, the target protein as depicted in Table 13, or an immunologically active fragment, variant or mutant thereof, is administered parenterally, usually by intramuscular or subcutaneous injection in an appropriate vehicle. Other modes of administration, however, such as oral, intranasal or intradermal delivery, are also acceptable.

Vaccine formulations will contain an effective amount of the active ingredient in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the biofilm peptide or fragment thereof in one or more doses. Multiple doses may be administered as is required to maintain a state of immunity to the bacterium of interest, e.g., L. intracellularis.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

To prepare a vaccine, the purified target protein as depicted in Table 13, fragment, or variant thereof, can be isolated, lyophilized and stabilized, as described above. The target protein as depicted in Table 13 may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane diamine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine formulation, or may be conjugated to proteins such as keyhole limpet hemocyanin (KLH) or human serum albumin (HSA) or other polymers.

The application of LI0447 or variant thereof, for vaccination of a mammal against colonization of L. interacellularis offers advantages over other vaccine candidates.

### Antibodies Specific for Target Proteins as Depicted in Table 13

Antibodies can be raised in animals (e.g., rabbits or other rodents), in eggs of hyperimmunized poultry, can be obtained from antibody-producing cell lines, or by other antibody-producing methods generally known in the art. Such antibodies can then be administered to the host animal. This method of passive immunization is the method of choice when an animal is already infected, and there is no time to allow the natural immune response to be triggered. It also can be beneficial to use this method in immune-compromised animals. Administered antibodies against L. intracellularis can in these cases bind directly to the bacteria. This has the advantage that it immediately decreases or stops L. intracellularis growth.

### Formulations and Methods of Administration

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

The L. intracellularis immunogenic compositions and vaccines of the invention may be formulated as pharmaceutical compositions and administered to a mammalian host, such as a pig or horse, in a variety of forms adapted to the chosen route of administration, i.e., orally, intranasally, intradermally or parenterally, by intravenous, intramuscular, topical or subcutaneous routes.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the animal's diet. For oral therapeutic administration, the active composition may be combined with one or more excipients and used in the form of ingestible tablets, buccal tablets, troches, capsules, elixirs, suspensions, syrups, wafers, and the like. Such compositions and preparations should contain at least 0.1% of active composition. The percentage of the compositions and preparations may, of course, be varied and may conveniently be between about 2 to about 60% of the weight of a given unit dosage form. The amount of active composition in such therapeutically useful compositions is such that an effective dosage level will be obtained.

When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. Of course, any material used in preparing any unit dosage form should be pharmaceutically acceptable and substantially non-toxic in the amounts employed. In addition, the active composition may be incorporated into sustained-release preparations and devices.

The active composition may also be administered intravenously or intraperitoneally by infusion or injection. Solutions of the active composition or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active composition in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following examples are intended to illustrate but not limit the invention.

### EXAMPLE 1

### Comparative Transcriptional Analysis of Homologous Pathogenic and Non-Pathogenic Lawsonia intracellularis Isolates in Infected Porcine Cells

Lawsonia intracellularis is the causative agent of proliferative enteropathy. This disease affects various animal species, including nonhuman primates, has been endemic in pigs, and is an emerging concern in horses. Non-pathogenic variants obtained through multiple passages in cell culture do not induce disease, but bacterial isolates at low passage induce clinical and pathological changes. We hypothesize that genes differentially expressed between pathogenic (passage 10) and non-pathogenic (passage 60) homologous L. intracellularis isolates encode potential bacterial virulence factors. The present study used high-throughput sequencing technology to characterize the transcriptional profiling of a pathogenic and a non-pathogenic homologous L. intracellularis variant during in vitro infection. A total of 401 genes were exclusively expressed by the pathogenic variant. Plasmid-encoded genes and those involved in membrane transporter synthesis (e.g., ATP-binding cassette), adaptation and stress response (e.g., transcriptional regulators) were the categories mostly responsible for this wider transcriptional landscape. The entire gene repertoire of plasmid A was repressed in the non-pathogenic variant suggesting its relevant role in the virulence phenotype of the pathogenic variant. Of the 319 genes which were commonly expressed in both pathogenic and non-pathogenic variants, no significant difference was observed by comparing their normalized transcription levels (fold change±2; p<0.05). Unexpectedly, these genes demonstrated a positive correlation (r²=0.81; p<0.05), indicating the involvement of gene silencing (switching off) mechanisms to attenuate virulence properties of the pathogenic variant during multiple cell passages. Following the validation of these results by reverse transcriptase-quantitative PCR using ten selected genes, the present study represents the first report characterizing the transcriptional profile of L. intracellularis. The complexity of the virulence phenotype was demonstrated by the diversity of genes exclusively expressed in the pathogenic isolate. The results support our hypothesis and provide the basis for prospective mechanistic studies regarding specific roles of target genes involved in the pathogenesis, diagnosis and control of proliferative enteropathy.

Introduction

Lawsonia intracellularis is a fastidious intracellular bacterium and the etiologic agent of proliferative enteropathy (PE), an intestinal hyperplasic disease characterized by thickening of the mucosa of the intestine due to enterocyte proliferation. Cell proliferation is directly associated with bacterial infection and replication in the intestinal epithelium. As a result, mild to severe diarrhea is the major clinical sign described in infected animals. Since the 1990s, PE has been endemic in swine herds and has been occasionally reported in various other species, including nonhuman primates, wild mammalians and ratite birds. Outbreaks among foals began to be reported on breeding farms worldwide within the last decade. Therefore, PE is now considered an emerging disease in horses.

Although PE was first reported in 1931, the causative bacterium was primarily isolated only in 1993 using rat small intestinal cells (IEC-18) in strict microaerophilic environmental conditions. Since then, various cell lines have supported L. intracellularis growth in vitro, including insect and avian cell lines. To date, growth of the bacteria in axenic (cell-free) media has not been reported. Regardless of the cell type, the dynamics of the infection in vitro requires actively dividing cells in a microaerophilic atmosphere with the peak of infection at six to seven days post-inoculation. McOrist et al (1995) chronologically described the dynamics of the infection and bacterial replication in intestinal porcine epithelial cells (IPEC-J2). Most events closely resembled those observed at the cellular level in infected animals, including multiplication of the bacteria freely in the cell cytoplasm. While the dynamics of the infection have been well-characterized, little is known so far about the genetic basis for the virulence, pathogenesis or physiology of L. intracellularis.

Spontaneous attenuated isolates obtained through multiple passages in cell culture have not been successful at inducing typical PE lesions or reversing its virulence in experimentally-infected pigs. Conversely, bacterial isolates at low passage induce clinical and pathological changes typical of PE. Various standard DNA-based typing techniques, such as pulsed field gel electrophoresis (PFGE), multilocus sequence typing (MLST) and variable number tandem repeat (VNTR) have shown identical genotypes in both pathogenic (low passage) and non-pathogenic (high passage) variants. As a result, we believe their phenotypic properties occur at the transcriptional level. Bacterial genes differentially expressed between pathogenic (low passage) and non-pathogenic (high passage) homologous isolates have not been reported and this information may help to elucidate genes encoding the major bacterial virulence factors involved in the pathogenesis of PE.

We hypothesize that genes differentially expressed between pathogenic (passage 10) and non-pathogenic (passage 60) homologous L. intracellularis isolates encode potential bacterial virulence factors. High-throughput technology (RNA-seq) was used to characterize and compare the transcriptional profile of a pathogenic and a non-pathogenic variant. Plasmid-encoded genes, regulatory factors and ATP-binding cassette (ABC) transporters associated genes were important for contributing to the wider transcriptional landscape observed in the pathogenic isolate. Additionally, the present study provided novel information for studying specific mechanisms of target genes and their potential usefulness for the diagnosis and control of PE.

Results and Discussion

Whole-transcriptome profiling of bacterial organisms has been widely studied to understand global changes in gene expression in vitro and in vivo. Hybridization-based approaches have been successfully applied for years, but it has limited use in obligate intracellular bacteria since it needs a reference purified sample from cultivated bacteria. High-throughput technologies have overcome these limitations by providing high resolution data to describe the bacterial transcripts in different experimental conditions. The present study used RNA-seq to qualitatively and quantitatively characterize the transcriptional profile of pathogenic and non-pathogenic homologous L. intracellularis isolates during in vitro infection. Since this is the first comprehensive gene expression analysis regarding this organism, the findings are presented and discussed in a comparative pathogenomic approach based on the information available from other related bacterial organisms.

Mapping and Differential Expression

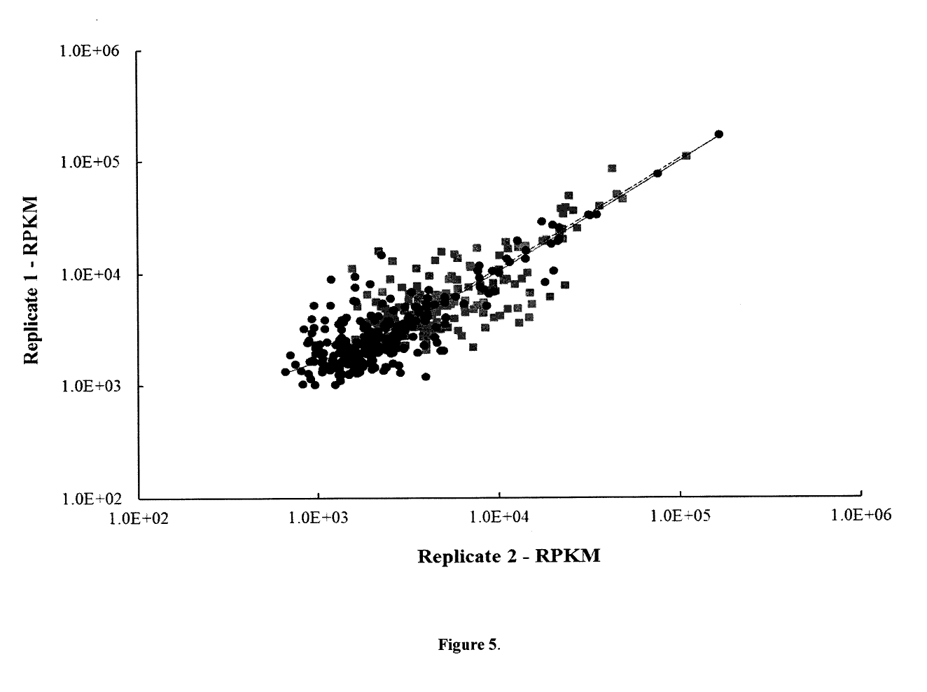

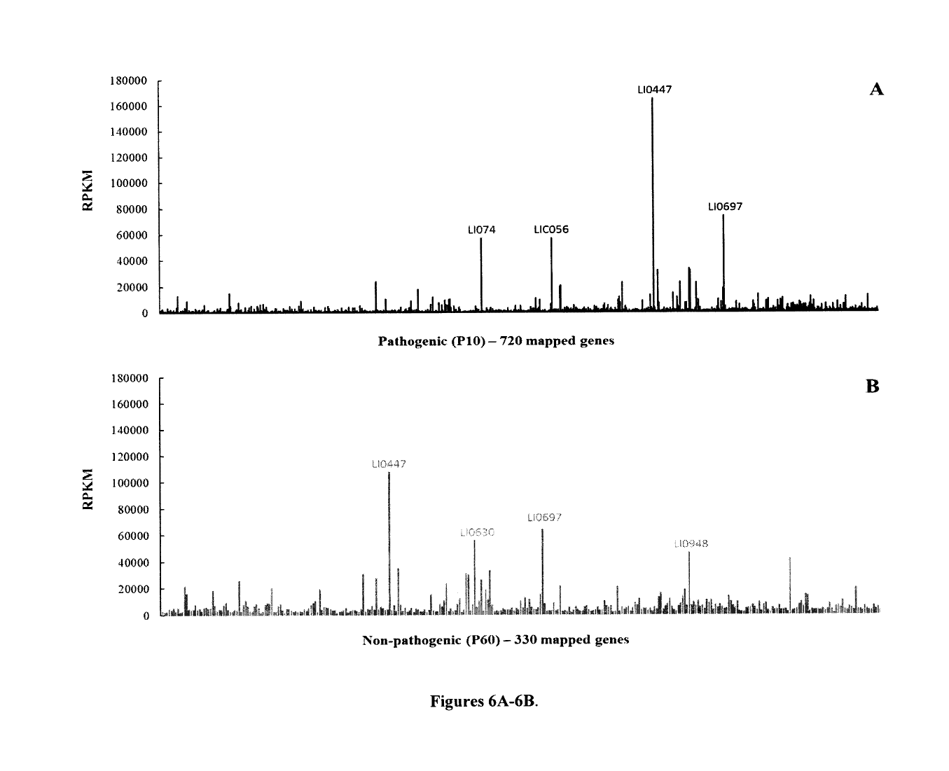

The sequence reads representing the RNA transcripts derived from the pathogenic and non-pathogenic homologous L. intracellularis PHE/MN1-00 isolates were mapped onto the complete genome sequence of the same bacterial strain available at the National Center for Biotechnology Information (NCBI) (accession: NC_{—}008011). The circular L. intracellularis genome has 1,719,014 base pairs (bp) comprised of one chromosome (1,457,619 bp) and three plasmids (plasmid A: 27,048 bp, plasmid B: 39,794 by and plasmid C: 194,553 bp). From a total of 1,391 computationally predicted genes in the annotated PHE/MN1-00 genome, 1,340 are protein coding. Combining the pathogenic and non-pathogenic transcript reads, 731 protein coding genes were mapped onto the reference DNA sequence of the L. intracellularis PHE/MN1-00 isolate. Sequence reads mapped against the bacterial genome were used to quantify the gene expression levels based on the number of reads per kilobase of coding sequence per million mapped reads (RPKM). The expression data were sufficiently reproducible by showing a positive correlation between the two biological replicates of the pathogenic (r²=0.86) and non-pathogenic (r²=0.81) variants which was obtained using a linear regression model (p<0.05). Genes with low expression levels showed lesser agreement between replicates (FIG. 5). This observation was reported using Illumina® RNA-seq data from human brain RNA and using ABI SOLiD™ platform in a transcriptome study of Neisseria gonorrhoeae. Following the default parameters of the CuffDiff software (see Material and Methods), our study used ten sequenced reads as the minimum number of alignments in a locus needed to conduct agreement and comparison testing within and between the biological replicates.

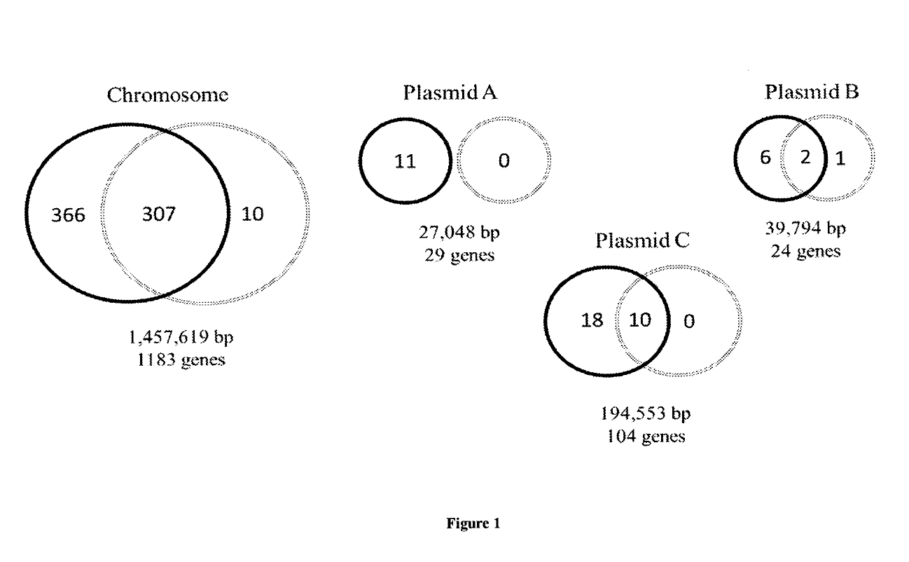

A total of 720 and 330 genes were expressed by the pathogenic and non-pathogenic variants, respectively (FIGS. 6A-6B). The wider transcriptional landscape observed in the pathogenic variant was characterized by 401 genes uniquely expressed by this variant. Genes with the highest transcription levels according to their biological function categories are shown in Table 1. Only 11 genes were expressed exclusively by the non-pathogenic variant (Table 2). Differential mapping and distribution of the expressed genes into the L. intracellularis chromosome and its three plasmids are summarized in FIG. 1. Plasmid-encoded genes significantly contributed to this broader profile of gene expression exhibited by the pathogenic isolate. The entire transcriptional repertoire of the plasmid A was suppressed in the non-pathogenic isolate suggesting its potential contribution in the L. intracellularis virulence. Transcription factors located in bacterial chromosomes have been shown to positively regulate virulence factors in the plasmid of Shigella flexneri and Pseudomonas syringae. Our study also identified many regulatory factors exclusively expressed by the pathogenic L. intracellularis variant which potentially regulate plasmid-encoded genes. Specific findings related to regulatory factors are discussed later.

A correlation between infectivity and loss of DNA contents from linear and/or circular plasmids during serial passages in vitro has been well established in Borrelia burgdorferi infections. Since there are a large number of different plasmids found in Borreliae genomes, their stability and loss in vitro vary between and within species. Although our study did not identify any transcriptional activities in the plasmid A of the non-pathogenic variant, the DNA contents of this plasmid were identical in both pathogenic and non-pathogenic variants (data not shown). Additionally, transcription levels were detected in three and ten genes present in the plasmid B and C of the non-pathogenic variant, respectively (FIG. 1). These findings demonstrated the occurrence of transcriptional activities and, consequently, the presence of these two plasmids in this variant. These observations support the hypothesis that global changes in gene expression are able to drive the loss of the virulence phenotype in cultivated L. intracellularis without altering the genomic DNA.

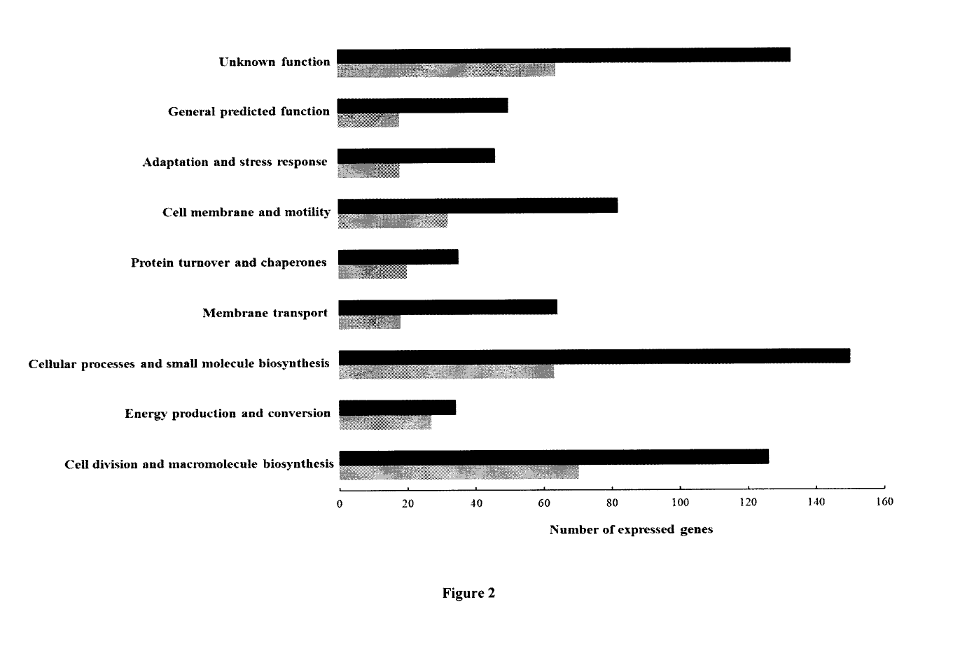

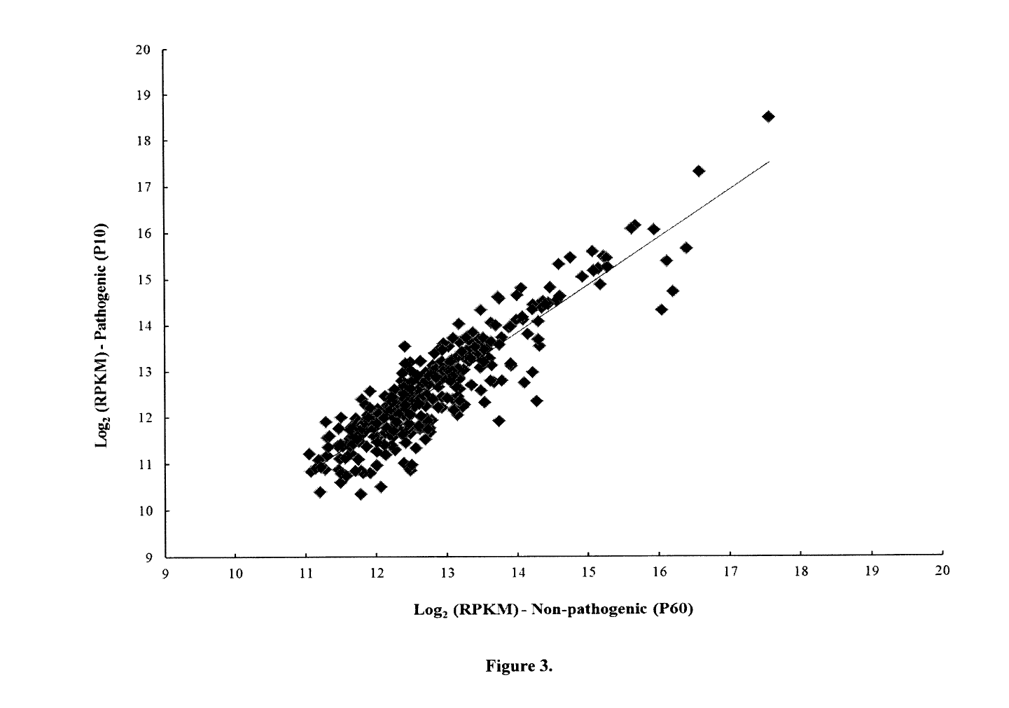

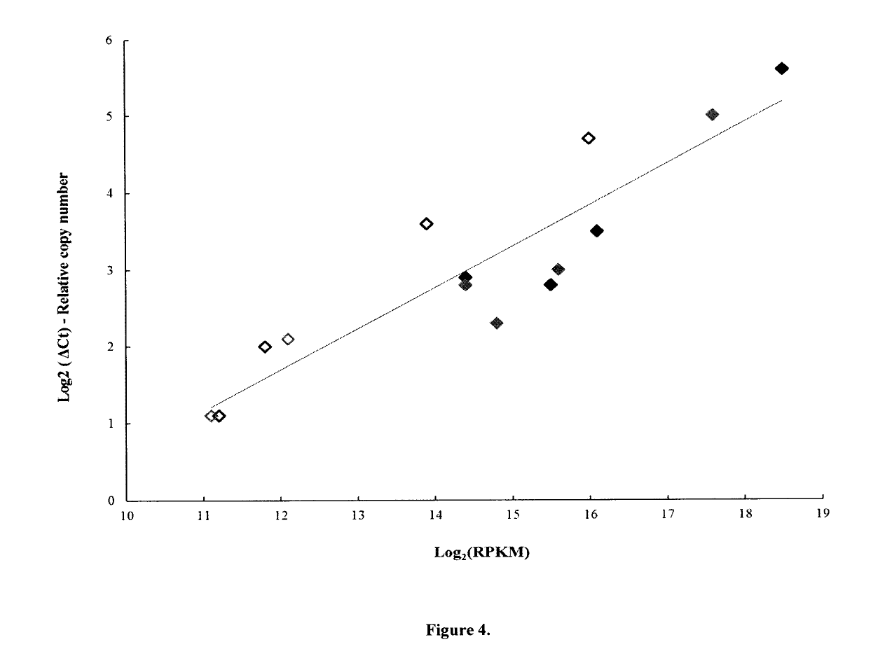

Narrowing the analysis to the putative biological gene functions, the number of genes expressed by the pathogenic variant was consistently higher throughout the functional categories (FIG. 2). The transcriptional landscape was most significantly reduced in those genes involved in the membrane transport (72%), general predicted function (64%), cell membrane and motility (61%) and adaptation and stress response (61%) categories. In comparing the transcription levels of all 319 genes commonly expressed in both the pathogenic and non-pathogenic variants, there was no significant difference (fold change ±2; adjusted p-value <0.05—Table 4). When the expression levels from both variants were plotted against each other (FIG. 3), log₂ (RPKM) values unexpectedly showed a positive correlation (0.809) in a linear regression model. These results revealed the importance of gene silencing (switching off) mechanisms to attenuate virulence properties of L. intracellularis throughout passages in vitro.

Regulation of gene expression during in vitro cultivation has been widely reported in various bacterial organisms. Bordetella pertussis switches off the expression of type III secretion system (TTSS) proteins in laboratory-adapted strains. One of the TTSS components, protein Bsp22, ceases to be expressed between passages three and four. Interestingly, these authors also showed the reversible re-expression of this protein after contact with the host in vivo. The expression of TTSS proteins has been demonstrated during L. intracellularis infection. However, our study failed to demonstrate consistent differences in the expression of TTSS proteins between pathogenic and non-pathogenic variants. Additionally, reversibility of virulence has not been reported in animals infected with laboratory-adapted L. intracellularis.

Transcriptional regulation has also been associated with mutations characterized by non-synonymous substitutions at the DNA level in Escherichia coli cultivated in glucose-limited medium for 20,000 generations. Regulatory changes in gene expression following by mutations in 12 lines of E. coli were driven by the specific environment in vitro evolving an ecological specialization in laboratory-adapted strains. Narrower transcriptional profiling of the non-pathogenic L. intracellularis passed 60 times in vitro was observed in our study; this could represent earlier stages of adaptation to a specialized in vitro environment. As in the case of E. coli, unnecessary functions that are costly to fitness in the in vitro condition might then be eliminated after hundreds or thousands of generations. According to ecological specialization models, organisms genetically adapted to one environment may lose fitness in other environments. Corroborating with this principle, the lack of reversible virulence in animals infected with laboratory-adapted L. intracellularis suggests that improving fitness in a specialized environment in vitro adversely affects performance in other complex substrates in vivo.

Gene expression data generated by RNA-seq were validated using quantitative reverse transcriptase PCR (qRT-PCR). The reliability of RNA-seq results was confirmed based on the relative quantification of 10 unlinked genes: four expressed in both variants, four expressed by the pathogenic and two expressed by the non-pathogenic (see Materials and Methods). The average log-transformed from two qRT-PCR replicates was plotted against the log₂ (RPKM) (FIG. 4). The transcript levels were positively correlated on a linear regression model (p-value <0.05; r²=0.827). Therefore, the RNA-seq data were consistent in quantitatively estimating the transcription levels of L. intracellularis during infection in vitro. The validation of RNA-seq data using qRT-PCR has also been previously reported in yeast and other bacterial transcriptomes.

Cell Division and Macromolecule Biosynthesis

Synthesis of proteins involved in cell division and ribosome biogenesis has been closely related to bacterial growth rate. From a total of 43 ribosomal protein-encoding genes present in the L. intracellularis reference genome, we identified 31 genes expressed by both the pathogenic and non-pathogenic variants and eight additional genes exclusively expressed by the pathogenic one. Furthermore, the operon responsible for orchestrating bacterial cell division that contained the FtsA and FtsZ genes was also identified in both variants (Table 4). The expression of RNA polymerase α and β subunits has also been associated with growth rate in that their synthesis increases in response to a rich nutrient medium (nutritional shift-up). All L. intracellularis genes encoding RNA polymerase subunits previously predicted from the annotated genome were expressed in both variants. Regardless of the pathogenic or non-pathogenic phenotype, these observations demonstrated that both variants grew at the same rate and were harvested during exponential phase at time of RNA harvesting (five days post-infection). Likewise, the results corroborate previous studies which described an increase in the number of Lawsonia-infected cells from two to seven days after infection. Adverse effects in bacterial growth have been related to the reduced expression of RNA polymerase subunits and ribosomal proteins in Neisseria gonorrhoeae cultivated in an oxygen-limited environment. The microaerophilic environment provided in our study was first described by Lawson et al (1993) and it has been shown to be an optimal atmosphere for cultivation of L. intracellularis.

Energy Production and Conversion

Pathogenic bacteria are frequently able to couple virulence pathways with general metabolic functions such as energy production, cellular signaling and molecular biosynthesis. The operon comprising F₀-F₁ ATP synthase subunits, which was expressed in both pathogenic and non-pathogenic L. intracellularis variants (Table 4), is essential for life in many bacterial species since it maintains pH homeostasis of the cell. Concomitantly, it plays a critical role in the acid stress response, specifically in enteric bacteria which encounter low pH conditions transiting through stomach and in the phagosome of host cells. Although we intended to harvest L. intracellularis freely multiplying in the cell cytoplasm (five days post-infection) which would fully represent its exponential growth phase, the identification of the F₀-F₁ operon in both variants suggests that a fraction of harvested intracellular bacteria was in the transient phagosome phase.

Along with these observations, the expression data also indicated the presence of free intracellular bacteria in the cell cytoplasm by detecting high transcriptional levels (Table 4) of rickettsia-like ATP/ADP translocase gene in both variants. This transmembrane protein was first identified in a few obligate intracellular bacteria, including Chlamydiales, Rickettsiales and amoebal symbionts, and more recently in L. intracellularis. A remarkable adaptation of these organisms to an intracellular microenvironment allows them to import cytoplasmic ATP generated in their hosts into the prokaryotic cell across the bacterial cell membrane. In an exchange mode, the bacterial ADP is exported back into the host cytosol. This exploitation of the host's energy pool is also referred to as energy parasitism. In agreement with the exponential growth phase of L. intracellularis, we also identified the expression of cytochrome bd operon in the pathogenic and non-pathogenic variant (Table 4). This oxygen reductase is used in the bioenergetic pathway in a variety of bacterial organisms under O₂-limited conditions.

Cellular Processes and Small Molecule Biosynthesis

Although L. intracellularis and other obligate intracellular bacteria depend on their hosts for certain nutrients (e.g. cytoplasmic ATP, see above), a wide spectrum of biosynthetic pathway-encoding genes are critical for fulfilling their essential functions. The 2-C-methyl-D-erythritol 4-phosphate (MEP) pathway is primarily involved in the biosynthesis of structural and functional isoprenoid molecules but the genes encoding the final two enzymes, ispG and lytB, have also been related to intracellular survival and induction of cellular immune response. Six genes of the Lawsonia MEP pathway previously predicted in the Kyoto encyclopedia of gene and genomes (KEGG) database were exclusively expressed by the pathogenic variant. However, the final remaining two enzymes (ispG and lytB) were transcribed by both the pathogenic and non-pathogenic variants. We suggest that ispG and lytB may be essential genes in L. intracellularis, as previously reported in several other bacterial pathogens. Based on this vital characteristic of the MEP pathway, it has recently been used as a drug target. Fosmidomicyn showed the ability to inhibit the second enzyme of the MEP pathway (Dxr) and has been used in clinical trials. Interestingly, our study identified expression levels of the Dxr-encoding gene (log₂ [RPKM]=10.1) exclusively in the pathogenic variant, suggesting its potential use for drug targeting.

Despite the similarities in the expression of ribosomal proteins and RNA polymerase subunits between pathogenic and non-pathogenic variants described previously, our study also identified the expression of relA gene exclusively in the pathogenic variant. This gene encodes an enzyme responsible for synthesizing guanosine tetraphosphate (ppGpp). This small signal molecule, also called stringent factor, is involved in various cellular processes and affects the control of growth rates by binding to RNA polymerase and reduces synthesis of ribosomal proteins and tRNA molecules during nutritional stress. Although the transcript reads of relA gene were exclusively identified in the pathogenic variant, its expression levels were relatively low (log₂ [RPKM]=9.7) suggesting that it was not sufficient to affect the expression of genes involved in macromolecule biosynthesis.

Based on the empirical observation that high-passage variants of L. intracellularis have higher growth rates than low passages in vitro, we believe that relA-dependent accumulation of ppGpp may control the bacterial growth rate at some point during the course of the infection in vitro. However, chronological transcriptional analysis is needed in order to elucidate this question. Supporting this speculation, Cooper et al (2003) demonstrated an increased fitness of laboratory-adapted E. coli (after 20,000 generations in vitro) associated with reduction in the concentration of ppGpp. These authors did not observe any mutation in the DNA sequence of relA gene. Similarly, the suppression of Lawsonia relA gene in the non-pathogenic variant was not associated with any mutations in the relA DNA sequence (data not shown).

The stringent response mediated by ppGpp also plays an important role in bacterial virulence, especially to adapt to conditions encountered in the intracellular host environment. A relA mutant strain of Listeria monocytogenes showed no virulence in vivo using murine infection models. However, the same study shows no difference between the wild type and ΔrelA mutant strain in their ability to escape from the phagosome and polymerize host cell actin in vitro using Caco-2 cells. Comparable phenotypic differences between in vitro and in vivo infections have also been observed in L. intracellularis. Low passage and high passage variants have similar abilities to infect cells in vitro but they demonstrated differences in pathogenic and non-pathogenic phenotypes in vivo.

Membrane Transport

The majority of genes encoding ABC transporters identified in the L. intracellularis genome were shown to be expressed in the pathogenic variant. From a total of 14 ABC transporter operons, 10 cassettes were specifically expressed by the pathogenic variant, including those involved in resistance to organic solvents, membrane transport of polyamines (spermidine), phosphates, nitrates, amino acids (glutamine and branched-chain), metallic cations (cobalt and nickel), lipopolysaccharides and lipoproteins. We specifically observed the highest expression levels in ABC uptake systems for amino acids (Table 1). Elevated transcription levels of the glutamine transport system (glnHPQ operon) were shown to be essential for virulence in Salmonella enterica Serovar Typhimurium and Streptococcus pneumoniae. Both studies demonstrated significant attenuation of the virulence in vivo using mouse models infected with mutant strains (ΔglnHPQ). Despite the attenuated phenotype, the mutant strain of S. enterica Typhimurium provided a protective immune response against challenge with wild-type. Additionally, the authors showed that glnHPQ operon is positively regulated by the sigma factor σ⁵⁴ (RpoN). This regulatory factor was also exclusively expressed by the pathogenic L. intracellularis variant in our study (Table 1). The expression of σ⁵⁴ and other transcription factors are discussed later in the subsection related to bacterial adaptation and stress response.

The in vitro growth kinetics of S. enterica Typhimurium and S. pneumoniae glnHPQ mutants were not affected in either broth or agar supplemented with glutamine or peptides. On the other hand, both studies showed reduced intracellular survival of these mutants in macrophage cell lines. There is no information regarding the intracellular survival of pathogenic or non-pathogenic L. intracellularis in infected macrophages and no impairment of intracellular survival has been reported in L. intracellularis continually cultivated using epithelial or fibroblastic-like cells. Although the IPEC-J2 cell culture media used in our study was not supplemented with glutamine, the glutamine ABC transporters were consistently expressed only by the pathogenic variant in the two glnHPQ operons present in the L. intracellularis genome.

The ABC transporter involving polyamine uptake (PotABCD operon) and uniquely expressed in the pathogenic variant at moderate levels (log₂ [RPKM]=11.1) has also been important for virulence in other bacterial organisms. Corresponding with the glutamine ABC transporters discussed above, mutations in the PotABCD operon of S. pneumoniae showed no effect on the growth rates in vitro, but the mutant strain showed significant attenuation of virulence within murine models regardless of the inoculation route.

The differential mapping of other bacterial transport systems including TTSS and general secretory (Sec) pathways was not consistently different between the pathogenic and non-pathogenic variants. The role of TTSS proteins has been well established at earlier stages of infection and has been required for the invasion of various bacterial organisms. Our study detected the intracellular expression of five of a total of 15 L. intracellularis genes involved in the synthesis of TTSS previously identified in the KEGG database. This unexpected intracellular expression of TTSS proteins was reported in S. enterica Typhimurium and plays a role in the survival of this organism within the Salmonella-containing vacuole.

The expression of genes encoding structural components of TTSS (YscN, YscO and YscQ) was previously identified by RT-PCR in three L. intracellularis isolates infecting rat small intestinal cells (IEC-18). However, the study did not provide information about the number of cell passages the isolates had undergone or the time points in which the bacterial RNA was harvested from the infected cell monolayers. Our study identified two (YscQ and YscN) of the three major TTSS components cited above. The YscQ gene was expressed in both the pathogenic and non-pathogenic variant and YscN was uniquely identified in the non-pathogenic variant (Table 2). These variable results regarding the expression of TTSS proteins suggest that further studies are required in order to elucidate the role of TTSS in L. intracellularis infection in vitro.

Protein Turnover and Chaperones

Chaperone-encoded genes (DnaK, DnaJ, GroEL and GroES) were highly expressed by the pathogenic and non-pathogenic variants. This observation corroborates the up-regulation of this gene category identified in a variety of bacterial organisms growing inside eukaryotic cells. Furthermore, bacterial chaperones are known to be essential for overcoming bacterial stress by ensuring the proper folding of proteins. Three additional genes (CbpA, CrpE and ClpA) involving the synthesis of chaperone molecules were exclusively expressed by the pathogenic variant and their specific biological functions remain to be determined.

Cell Membrane and Motility

A broader diversity of genes encoding cell wall molecules were observed in the pathogenic L. intracellularis variant, suggesting more extensive options for remodeling of the bacterial envelope compared with the non-pathogenic variant. This characteristic is important for altering the bacterial cell surface during different steps of the infectious process. The wider variety of this gene category in the pathogenic variant was predominantly comprised of genes encoding glycosyltransferase enzymes which have been implicated in posttranslational processing of proteins responsible for modifying the lipopolysaccharide composition. Modifications of the glycosylation composition of the cell wall were depicted in Campylobacter jejuni during its intestinal life cycle. These events were then considered part of the strategy used by the bacterium to resist and evade the host immune responses.

In the mapping of genes involved in the flagellar assembly pathway, there were no consistent differences between the pathogenic and non-pathogenic variants. From a total of 30 predicted genes in the KEGG database, ten were expressed in both variants (Table 4), eight were exclusively identified in the pathogenic and one was unique from the non-pathogenic variant (Table 2). Our study detected similar expression levels of the FITC gene in both variants (Table 4). The flagellin encoded by this gene has been well studied regarding its ability to induce an immune response. S. enterica Typhimurium expressed this protein in the membrane-bound compartment and it was then translocated into the host cytoplasm to be detected by cytosolic Nod-like receptors which are able to mount an innate immune response. Studies characterizing the immune response of cells infected with pathogenic and non-pathogenic L. intracellularis would be important to detect specific host factors associated with these two variants.

Adaptation and Stress Response

The number of genes encoding proteins involved in adaptation and stress response was one of the gene categories with significant reduction (61%) in the non-pathogenic variant (FIG. 2). Ten of the 28 genes uniquely expressed by the pathogenic variant in this category were transcriptional regulatory factors. These molecules regulate bacterial gene expression by acting globally or at specific DNA regions to active or repress transcription or modulate DNA topology. The σ⁷⁰ is the “housekeeping” sigma factor and has been required for cell growth in the majority of bacterial organisms. In agreement with this, we observed similar expression levels of σ⁷⁰ in the pathogenic and non-pathogenic variants (Table 4). Conversely, the global regulator σ⁵⁴ was uniquely expressed in the pathogenic variant (Table 1) and has been reported to control the transcription of virulence genes in a variety of bacteria species. Specifically in S. enterica Typhimurium, the role of σ⁵⁴ is to coordinate the transcription of the glutamine ABC transporter, glnHPQ operon, during the intracellular life cycle. The coincidental and exclusive co-expression of σ⁵⁴ and glnHPQ by the pathogenic variant supports the hypothesis that this sigma factor may also coordinate the glutamine uptake operon in L. intracellularis to ensure an adequate supply of this crucial amino acid during its intracellular life. Additionally, the consistently higher number of ABC transporter-encoding genes expressed by the pathogenic, but not the non-pathogenic, variant begs the question whether σ⁵⁴ is also able to positively regulate other ABC transporters (e.g. polyamines and branched-chain amino acid transporters). The obligate intracellular nature of L. intracellularis has imposed considerable limitations in elucidating this and other specific regulatory mechanisms. To date, the construction of recombinant plasmids appears to be an alternative model for overcoming these barriers.

A gene encoding the major transcription factor involved in the iron homeostasis (fur regulator) was uniquely expressed by the pathogenic variant (Table 1) and its transcription levels were confirmed by qRT-PCR (FIG. 4; Table 5). The fur regulator can act as either a repressor or an activator in a variety of bacterial organisms. The wider gene expression profile identified in the pathogenic variant of L. intracellularis suggests the role of fur as an activator in our experimental conditions. Supporting this hypothesis, fur-activated genes have been reported in other enteric pathogens (e.g. S. enterica Typhimurium) and bacteria that replicate freely in the host cytoplasm (e.g. L. monocytogenes). Although this transcription factor is typically related to the iron metabolism in response to iron availability, its role in the stress response and virulence in vivo has also been well established. For instance, fur mutants of L. monocytogenes and C. jejuni showed reduced virulence in experimental models. Furthermore, the in vitro growth rates in a fur mutant strain of Desulfovibrio vulgaris, one of the closest bacterial species genetically related to L. intracellularis, were not affected.

In addition to its role in virulence, fur acts in the regulation of genes involved in oxidative stress, such as superoxide dismutase (sod). However, the unique sod gene previously annotated in the L. intracellularis genome (sodC gene) was expressed at high levels by both pathogenic and non-pathogenic variants (Table 4). Its transcription levels were validated by qRT-PCR (FIG. 4; Table 5). This finding indicates that a fur-independent mechanism potentially regulates sodC expression in vitro. In agreement with this speculation, the literature describes fur regulation of sodA (Mn²⁺-containing sod) and sodB (Fe²⁺-containing sod) but not sodC (Cu—Zn²⁺-containing sod). In addition, fur mutation had no effect on the regulation of sodC in E. coli. Regardless of specific regulatory mechanisms, the expression of the sodC gene is critical for intracellular survival of pathogenic bacteria by catalyzing reactive host-derived superoxide radicals (O₂⁻) to hydrogen peroxide (H₂O₂) and oxygen (O₂). In our study, this mechanism also appears to be essential for both pathogenic and non-pathogenic L. intracellularis during in vitro infection. The gene (rubA) encoding rubredoxin 2 (Rb-2) was computationally predicted in the L. intracellularis genome and has been described to complement the catalytic activity of sodC against oxidative stress in Desulfovibrio species. Our study identified rubA as the second most commonly expressed gene by both pathogenic and non-pathogenic variants (Table 4). According to a model proposed by Lumppio et al (2001), the superoxide dismutase acts in the periplasm fraction and the Rb-2 neutralizes superoxide radicals in the bacterial cytoplasm.

Predicted/Unknown Function

Genes encoding hypothetical proteins comprise approximately 27.2% of the 1340 protein encoding genes previously predicted in the reference DNA sequence of the L. intracellularis PHE/MN1-00 isolate. This significant number becomes more evident within the plasmid A (55.2%), plasmid B (58.3%) and plasmid C (63.5%). Our study identified four genes encoding hypothetical proteins in the ten most highly expressed genes commonly identified in the pathogenic and non-pathogenic variants. Among all commonly expressed genes, the LI0447 gene demonstrated the highest transcription levels (Table 4) which was confirmed by qRT-PCR (FIG. 4; Table 5). The protein encoded by the LI0447 gene was computationally predicted to be a transmembrane protein with 50.7% identity at the amino acid level to other hypothetical proteins identified in the Mannheimia haemolytica genome (MHA_{—}1476—accession: ZP_{—}04978003.1). An autotransporter protein (LatA) was recently characterized as a prominent antigen during infection of IEC-18 cells with the L. intracellularis isolate LR189/5/83(Watson E, Clark E M, Alberdi M P, Inglis N F, Porter M, et al. (2011) A novel Lawsonia intracellularis autotransporter protein is a prominent antigen. Clin Vaccine Immunol.). However, the authors did not specify the number of in vitro passages that bacterial isolate had. Our study identified the gene encoding this protein, referred to as LI0649, in both the pathogenic and non-pathogenic variants (Table 4). Regarding the genes uniquely expressed by the pathogenic variant, the hypothetical protein encoded by the LI0259 gene was the third most highly expressed chromosomal gene (Table 1). This protein (accession: YP_{—}594636) has a conserved putative domain of 41 amino acids found in a wide range of bacteria and is noted as a regulatory factor included in the FmdB family.

From a total of 35 plasmid-encoded genes uniquely expressed by the pathogenic variant (FIG. 1), 19 genes were predicted to encode hypothetical proteins. Genes showing the highest transcription levels in the plasmids are summarized in Table 3. The LIC056 gene was the most highly expressed gene identified uniquely in the pathogenic variant and its transcription levels were confirmed by qRT-PCR (FIG. 4; Table 5). This gene encodes a predicted transmembrane protein containing a conserved autotransporter beta-domain. This class of proteins has been implied to mediate secretion by translocation of bacterial proteins across the outer membrane. Another intriguing observation was the expression of the LIC091 gene exclusively in the pathogenic variant (Table 3). This gene has been predicted to encode one of the largest proteins among all bacterial organisms containing 8746 amino acids. Eight different protein families have been proposed for this molecule including a transmembrane protein with 99.5% of its amino acid sequence belonging to an extracellular domain. Our study provides evidence regarding the potential role of genes encoding hypothetical proteins during L. intracellularis infection in vitro, but their specific biological functions remain to be elucidated. Additionally, the substantial number of these genes previously identified in the L. intracellularis genome and now associated with its transcriptional profiling suggests that this organism may adopt unique mechanisms of survival and pathogenesis among bacterial pathogens.

Conclusions

The present study is the first report characterizing the transcriptional profile of L. intracellularis and comparing the gene expression levels of a pathogenic and a non-pathogenic homologous isolate. The wider transcriptional landscape identified in the pathogenic variant was consistent throughout the gene categories and had significant contributions of plasmid-encoded genes. High expression levels of genes encoding ABC transporters and specific transcriptional regulators were uniquely identified in the pathogenic variant and suggest specific metabolic adaptation of L. intracellularis, including substrate acquisition that allows its efficient proliferation in the infected host. The dynamics of the genetic changes in laboratory-adapted bacterial organisms have developed a new ecological specialization which results in different bacterial phenotypes. In our study, the lack of selective pressure during multiple cell passages in vitro might be the reason for the narrower transcriptional profile observed in the non-pathogenic variant and loss of pathogenicity in vivo by gene silencing (switching off) mechanisms.

The diversity of genes exclusively expressed in the pathogenic variant and repressed in the non-pathogenic, including those involved in membrane transport, adaptation and stress response, justifies the complexity of the virulence phenotype which was attenuated probably due to a combination of mechanisms. In this regard, the wide gene expression analysis in our study was important to globally characterize both pathogenic and non-pathogenic phenotypes and to provide the basis for future mechanistic studies. Finally, the results support our hypothesis and open a new research field for studying target genes involved in the pathogenesis, diagnosis and control of PE.

Materials and Methods

Cell Culture and Infection in Vitro

The intestinal piglet epithelial cell line IPEC-J2 is a non-transformed columnar cell type derived from neonatal piglet mid jejunum (Berschneider H M (1989) Development of normal cultured small intestinal epithelial cell lines which transport Na and Cl. Gastroenterology 96: A41). The cells were maintained in T₇₅ cell culture flasks with Dulbecco's MEM/F12 nutrient mix (1:1) supplemented with 5% Fetal Bovine Serum, 5 ng/ml Epidermal Growth Factor (Sigma-Aldrich) and 5 ng/ml Insulin-Transferrin-Selenium mixture (BD Biosciences) without antibiotics at 37° C. in a humidified atmosphere of 5% CO₂, as previously described (McOrist S, Jasni S, Mackie R A, Berschneider H M, Rowland A C, et al. (1995) Entry of the bacterium ileal symbiont intracellularis into cultured enterocytes and its subsequent release. Res Vet Sci 59: 255-260).

L. intracellularis isolate PHE/MN1-00 (ATCC PTA-3457) previously isolated from a pig with the hemorrhagic form of PE was used for passage 6 (pathogenic variant) and 56 (non-pathogenic variant) in cell culture. The pathogenic and non-pathogenic properties of these two variants were confirmed by experimental inoculation in pigs, performed in a previous study (Vannucci F A, Beckler D, Pusterla N, Mapes S M, Gebhart C J (2012) Attenuation of virulence of Lawsonia intracellularis after in vitro passages and its effects on the experimental reproduction of porcine proliferative enteropathy. (Accepted) [In press] Veterinary Microbiology). Pure culture of the bacteria was thawed and grown in IPEC-J2 for three continuous passages in order to allow the bacteria to recover from frozen storage. Cell culture flasks T₇₅ containing 30% confluent IPEC-J2 cell monolayer were infected with 10³ pathogenic (passage 10) and non-pathogenic (passage 60) L. intracellularis organisms derived from the isolate (PHE/MN1-00). Inoculated cultures were placed in an anaerobic chamber, which was evacuated to 500 mmHg and refilled with hydrogen gas. Infected cultures were then incubated for seven days in a Tri-gas incubator with 83.2% nitrogen gas, 8.8% carbon dioxide, 8% oxygen gas and a temperature of 37° C. (Lawson G H, McOrist S, Jasni S, Mackie R A (1993) Intracellular bacteria of porcine proliferative enteropathy: cultivation and maintenance in vitro. J Clin Microbiol 31: 1136-1142).

The infection was monitored daily by counting the number of heavily infected cells (HIC) using immunoperoxidase staining with polyclonal antibody specific for L. intracellularis, as previously described (Guedes R M, Gebhart C J, Deen J, Winkelman N L (2002) Validation of an immunoperoxidase monolayer assay as a serologic test for porcine proliferative enteropathy. J Vet Diagn Invest 14: 528-530). A parallel infection was also monitored using 16-well tissue culture plates, as described in our previous study (Vannucci F A, Wattanaphansak S, Gebhart C J (2012) An Alternative Method for Cultivation of Lawsonia intracellularis. J Clin Microbiol 50: 1070-1072). The inoculated doses in this parallel monitoring system were proportional to those used in the T₇₅ flasks according to the number of IPEC-J2 cells previously passed on the day before the infection. The monitoring was achieved by counting the number of HIC daily in eight wells (replicates) infected with the pathogenic and eight with the non-pathogenic variant. Cells containing 30 or more intracellular bacteria were considered to be HIC (McOrist S, Mackie R A, Lawson G H (1995) Antimicrobial susceptibility of ileal symbiont intracellularis isolated from pigs with proliferative enteropathy. J Clin Microbiol 33: 1314-1317; Wattanaphansak S, Singer R S, Gebhart C J (2009) In vitro antimicrobial activity against 10 North American and European Lawsonia intracellularis isolates. Vet Microbiol 134: 305-310). Additionally, quantitative PCR (qPCR) based on the copy numbers of aspartate ammonia lyase gene of L. intracellularis was performed daily as a second parameter of monitoring, as described elsewhere (Vannucci F A, Wattanaphansak S, Gebhart C J (2012) An Alternative Method for Cultivation of Lawsonia intracellularis. J Clin Microbiol 50: 1070-1072). Direct counting of infected cells and qPCR were used to confirm the exponential phase of the bacterial growth and to standardize the amount of L. intracellularis used as starting material for RNA isolation. All procedures used in the present study were approved by the Institutional Sponsored Projects Administration of the University of Minnesota.

RNA Isolation and Enrichment

On the fourth continuous passage, the infection was passed using two replicates from each variant (pathogenic and non-pathogenic) and a total of four infected monolayers were harvested. A negative control using non-infected IPEC-J2 cells was conducted by treating the monolayers with sterile complete media. On day five post-inoculation the supernatants were removed and the infected monolayers were washed with RNAprotect® Bacterial Reagent (Qiagen). The infected cells were scraped and passed through a 20-gauge needle five times. Total RNA were extracted using RNeasy® Mini Kit (Qiagen) with an additional step for removing residual DNA using DNase I (Qiagen). Total RNA were assessed by a NanoDrop ND-8000 Spectrophotometer and Agilent Bioanalyzer for quality and integrity. Only samples with RNA Integrity Numbers (RIN)≧8.0 were used in subsequent mRNA purification steps.

The bacterial mRNA was enriched from the total extracted RNA (mixture of cells and bacterial RNA) by subtractive hybridization using MicrobEnrich™ and MicrobExpress™ kits (Ambion Inc.). Briefly, oligonucleotides specific to the mammalian RNAs (18S rRNA, 28S rRNA and polyadenylated mRNAs) and to the bacterial ribosomal RNA (16S and 23S) were hybridized and captured by magnetic beads. Equal amounts of input RNA were used following the manufacturer's instructions for both pathogenic and non-pathogenic L. intracellularis variants. Internal controls provided in both kits were performed in all enrichments. The effectiveness of rRNA depletions was evaluated using an Agilent Bioanalyzer 2100 (mRNA Nano Series Assay).

Library Preparation and Sequencing

The library preparation and sequencing were conducted in the core facility of the Biomedical Genomics Center at the University of Minnesota. Briefly, 100 ng of the enriched mRNAs from both pathogenic and non-pathogenic L. intracellularis variants were fragmented and the first and second strand sDNAs were synthetized and ends repaired. The cDNA template was enriched by PCR and validated using High Sensitivity Chip on the Agilent2100 Bioanalyzer. Following quantification of the cDNA generated for the library using PicoGreen Assay, the samples were clustered and loaded on the Illumina® Genome Analyzer GA IIx platform which generated on average 6,190,522 single reads with 76 bp. Base calling and quality filtering were performed following the manufacturer's instructions (Illumina® GA Pipeline).

Analysis and Mapping the Sequence Reads

Data analysis including quality control, trimming and mapping were performed in the Galaxy platform (Giardine B, Riemer C, Hardison R C, Burhans R, Elnitski L, et al. (2005) Galaxy: a platform for interactive large-scale genome analysis. Genome Res 15: 1451-1455). Initially, FastQC tool was applied on the raw sequence data followed by FastQ Trimmer (Blankenberg D, Gordon A, Von Kuster G, Coraor N, Taylor J, et al. (2010) Manipulation of FASTQ data with Galaxy. Bioinformatics 26: 1783-1785). Using these tools, ten base pairs were trimmed from the 5′end and six from the 3′end. As a result, trimmed sequences containing 60 by were used in the gene expression analysis. The sequence reads showed more than 25 phred quality score (Ewing B, Hillier L, Wendl M C, Green P (1998) Base-calling of automated sequencer traces using phred. I. Accuracy assessment. Genome Res 8: 175-185) and were then mapped onto the L. intracellularis isolate PHE/MN1-00 reference genome obtained from the NCBI database using Bowtie short read aligner (Langmead B, Trapnell C, Pop M, Salzberg S L (2009) Ultrafast and memory-efficient alignment of short DNA sequences to the human genome. Genome Biol 10: R25), with no more than two mismatches.

The number of reads that were mapped within each annotated coding sequence (CDS) was calculated in order to estimate the level of transcription for each gene. The Cufflinks tool was used to estimate the relative abundances of the transcript reads in each gene (Trapnell C, Williams B A, Pertea G, Mortazavi A, Kwan G, et al. (2010) Transcript assembly and quantification by RNA-Seq reveals unannotated transcripts and isoform switching during cell differentiation. Nat Biotechnol 28: 511-515). For comparison of the expression levels between the pathogenic and non-pathogenic L. intracellularis variants, the read counts were normalized based on the number of reads per kilobase of coding sequence per million mapped reads (RPKM).

Differential Expression Analysis

Following the quantitative analysis of expressed genes, the differential expression between pathogenic and non-pathogenic variants was assessed using the CuffDiff tool (Trapnell C, Williams B A, Pertea G, Mortazavi A, Kwan G, et al. (2010) Transcript assembly and quantification by RNA-Seq reveals unannotated transcripts and isoform switching during cell differentiation. Nat Biotechnol 28: 511-515). The total read count was determined for each gene by combining data from the two replicate sequencing runs. RPKM values were expressed in log₂ (RPKM) to allow for the statistical comparison of transcription levels. As a result, log₂-fold change in abundance of each transcript was obtained by log₂ (RPKM_{[pathogenic]}/RPKM_{[non-pathogenic]}). P-values were calculated and adjusted for multiple comparisons using false discovery rate (FDR) (Benjamini Y, Hochberg Y (1995) Controlling the False Discovery Rate: A Practical and Powerful Approach to Multiple Testing. Journal of the Royal Statistical Society Series B 57: 289-300). Significant differential expression was determined in genes with FDR-adjusted p-values<0.05 and fold change±2 in the comparison of transcription levels between pathogenic and non-pathogenic variants.

Quantitative Reverse Transcriptase PCR

The validation of the expression data identified by RNA-seq was performed by qRT-PCR from a specific set of genes: LI0005 (superoxide dismutase); LI0035 (fur regulator); LI0447 (hypothetical protein); LI0614 (thioredoxin); LI0902 (outer membrane protein); LIA017 (Fe—S oxidoreductase); LIC056 (hypothetical protein); LIB024 (chromosome partitioning ATPase); LI0825 (lipid A core-O-antigen ligase) and LI0959 (30S ribosomal protein S10). Specific primers were designed using Roche Universal Probe Library (UPL) generating a UPL probe number (Table 5). RNA samples were synthesized to first-strand cDNA using SuperScript® II RT (Invitrogen, Carlsbad, Calif.). Duplicate qRT-PCR reactions from each primer probe set were validated by five serial dilutions of cDNA on the ABI7900HT instrument (Applied Biosystems, Foster City, Calif.). After validation, quantitative PCR was performed in duplicates using 15 ng of cDNA per sample with the following conditions: 60° C. for 2 min, 95° C. for 5 min and 45 cycles (95° C./10 sec and 60° for 1 min). Averages of relative transcriptional levels were calculated and log₂ transformed in order to be compared to the RNA-seq expression levels. Linear regression model was used to evaluate the correlation between average RPKM and qRT-PCR data.

### EXAMPLE 2

### Laser Microdissection Coupled with RNA-seq Analysis of Porcine Enterocytes Infected with an Obligate Intracellular Pathogen (Lawsonia intracellularis)

Abstract

Background & Aims: Lawsonia intracellularis is an obligate intracellular bacterium and the etiologic agent of proliferative enteropathy. The disease is endemic in pigs, emerging in horses and has been described in various other species including nonhuman primates. Cell proliferation is associated with bacterial replication in enterocyte cytoplasm, but the molecular basis of the host-pathogen interaction is unknown. We used laser capture microdissection coupled with RNA-seq technology to characterize the transcriptional responses of infected enterocytes and the bacterial interactome.

Methods: Three thousand each of Lawsonia-infected and non-infected enterocytes were microdissected. Amplified cDNA prepared by random priming was sequenced using an Illumina® platform. Sequence reads were mapped against porcine and L. intracellularis reference genomes to characterize the host transcriptome and the bacterial interactome.

Results: Proliferative enterocytes was associated with activation of transcription, protein biosynthesis and genes acting on the G₁ phase of the host cell cycle (Rho family). The lack of differentiation in infected enterocytes was demonstrated by the repression of membrane transporters related to nutrient acquisition. The activation of the copper uptake transporter by infected enterocytes was associated with high expression of the Zn/Cu superoxide dismutase by L. intracellularis. This suggests that the intracellular bacteria incorporate intracytoplasmic copper and express a sophisticated mechanism to cope with oxidative stress.

Conclusions: The feasibility of coupling microdissection and RNA-seq was demonstrated by characterizing the host-bacterial interactome from a specific cell type in a heterogeneous tissue. High expression of L. intracellularis genes encoding hypothetical proteins and activation of host Rho genes infers the role of unrecognized bacterial cyclomodulins in the pathogenesis of proliferative enteropathy.

Introduction

Cell proliferation concomitant with bacterial infections has been associated with carcinogenesis in chronic diseases caused by Helicobacter pylori, Salmonella typhi and Citrobacter rodentium. Proliferative changes resulting in a hyperplastic but non-carcinogenic process is induced in Lawsonia intracellularis and Bartonella spp. infections. The inflammatory mediators generated during the chronic gastritis in H. pylori infections have been related with oxidative DNA damage and cell transformation. However, other gram negative pathogenic bacteria (e.g. L. intracellularis and C. rodentium) are able to promote enterocyte proliferation with minimal inflammatory responses. C. rodentium induces proliferation of mouse colonic enterocytes when it attaches to the apical membrane. L. intracellularis escapes from the endosome after internalization and multiplies freely in the cytoplasm of undifferentiated intestinal crypts promoting their proliferation and progressive replacement of the differentiated intestinal epithelium by immature infected enterocytes. These unusual pathological changes characterized by the presence of a large number of intracellular bacteria and proliferation of enterocytes suggest that L. intracellularis has adopted mechanisms of survival and pathogenesis that are unique among bacterial pathogens. To date, hypotheses and speculations have been discussed regarding the pathogenesis of this infection but, the underlying mechanisms by which L. intracellularis induces proliferative changes have not been addressed.

Lawsonia intracellularis is an obligate intracellular bacterium and the etiologic agent of proliferative enteropathy (PE). Mild to severe diarrhea is the major clinical sign described in infected animals and is directly associated with cell proliferation and replication of the bacteria in the intestinal epithelium. PE is endemic in swine herds, an emerging disease in horses and has been reported in various other species, including non-human primates, wild mammals and ratite birds.

The disease was first reported in 1931 but, due to its fastidious properties, porcine L. intracellularis was first isolated in 1993 using rat small intestinal cells cultured in a strictly defined microaerophilic environment. Since then, the dynamics of infection in vitro and in vivo have been well-characterized. While proliferative changes in vivo follow an increase in the number of intracellular bacteria, the bacterium is not able to induce proliferation in infected cells in vitro. This intriguing observation associated with the fastidious properties of this bacterium has limited the studies on the pathogenesis of L. intracellularis. Additionally, the adaptation of this microaerophilic, but obligate intracellular organism to grow freely in the cytoplasm of metabolically active enterocytes suggests that this organism has properties that are novel and unique among bacterial pathogens.

In the present study, we hypothesized that genes expressed by L. intracellularis in the host cytoplasm are capable of inducing proliferation and preventing differentiation of immature enterocytes by altering cell cycle-associated pathways. We established a method integrating laser capture microdissection (LCM) and RNA-seq technology to characterize the host transcriptome and constructed the bacterial interactome in vivo. Activation of transcription, protein synthesis and Rho family genes in infected enterocytes characterized the transcriptional mechanisms involved in the cell proliferation. The ability of L. intracellularis in preventing enterocyte differentiation and maturation was proved by the consistent down-regulation of apical membrane transporters related to nutrient acquisition by infected enterocytes. The bacterial interactome showed a high level of expression of a sophisticated oxidative protection mechanism which involves redox enzymes and a rubrerythrin-rubredoxin operon (rubY-rub). Rho genes expressed by the host and associated with the high expression of bacterial genes encoding hypothetical proteins implies a potential role of unrecognized bacterial effector proteins that modulate eukaryotic cell cycle (cyclomodulins) in the pathogenesis of PE.

Materials and Methods

Experimental Infection

Twelve Duroc-Landrace cross pigs at 3-weeks-of-age were divided into two groups: infected and control (n=6/group). The animals were obtained from a herd with no history of PE and each treatment group was housed in a different pen. Prior to the study, blood and fecal samples from all animals were collected and tested for L. intracellularis-specific antibodies by immunoperoxidase monolayer assay (IPMA) and for the presence of Lawsonia DNA in the feces in order to confirm their negative status. The animals were allocated in the isolation barns at the College of Veterinary Medicine of the University of Minnesota and fed with non-medicated nursery feed and water ad libitum. All procedures were approved by the Institutional Animal Care and Use Committee of the University of Minnesota. The infected group was orally inoculated with 30 ml of L. intracellularis culture at 10⁸ organisms per ml. The non-infected group was orally treated with sodium-phosphate-glutamate (SPG) solution. Fecal samples were collected every other day and analyzed by qPCR for the presence L. intracellularis DNA (Pusterla N, Mapes S, Rejmanek D, et al. Detection of Lawsonia intracellularis by real-time PCR in the feces of free-living animals from equine farms with documented occurrence of equine proliferative enteropathy. J Wildl Dis 2008;44:992-8).

Laser Capture Microdissection

Five serial 8-μm frozen sections were cut at −20° C. using RNAse-free blades and mounted on StarFrost® RNAse-free slides (Fisher Scientific). The first and fifth cryosections (reference slides) were evaluated by immunohistochemistry (IHC) using the streptavidin method with polyclonal antibodies specific for L. intracellularis (Guedes R M, Gebhart C J. Preparation and characterization of polyclonal and monoclonal antibodies against Lawsonia intracellularis. J Vet Diagn Invest 2003; 15:438-46). In these two reference slides the levels of infection were assessed based on the amount of positively labeled antigen present in the intestinal sections: Grade 0 (−)=no positive antigen labeled; Grade 1 (+)=one isolated focal area of antigen labeled; Grade 2 (++)=multi-focal areas of antigen labeled; Grade 3 (+++)=majority of the mucosa has positive antigen labeled; and Grade 4 (++++)=all of the mucosa has positive antigen labeled (Guedes R M, Gebhart C J. Comparison of intestinal mucosa homogenate and pure culture of the homologous Lawsonia intracellularis isolate in reproducing proliferative enteropathy in swine. Vet Microbiol 2003; 93:159-66). The series of slides containing the two flanking pairs of reference slides most severely infected (grade 3 or 4) was selected for LCM from each animal. Similar anatomic portions of the ileum were collected from the control group and stained by IHC to confirm the negative status of the samples. One non-infected series of cryosections was selected for LCM from the similar anatomic portion of those selected in the infected group.

### Results

Technical Characterization of the LCM Coupled with RNA-seq Analysis

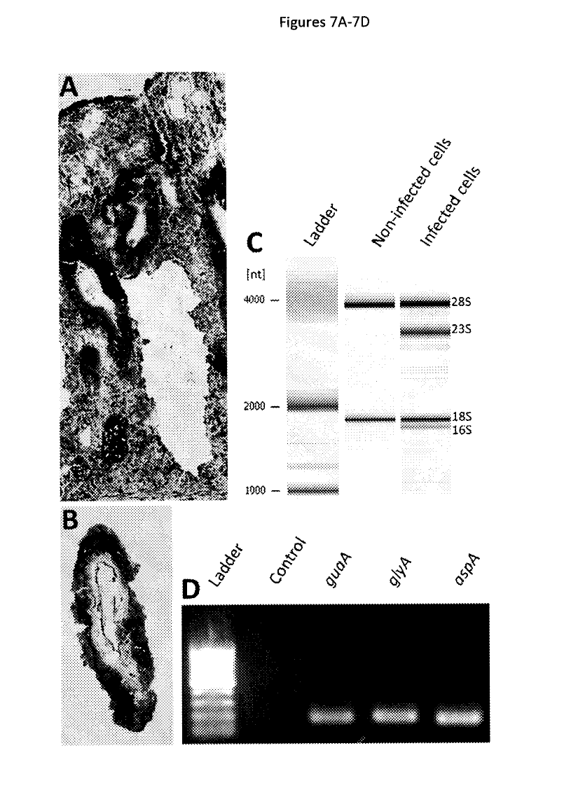

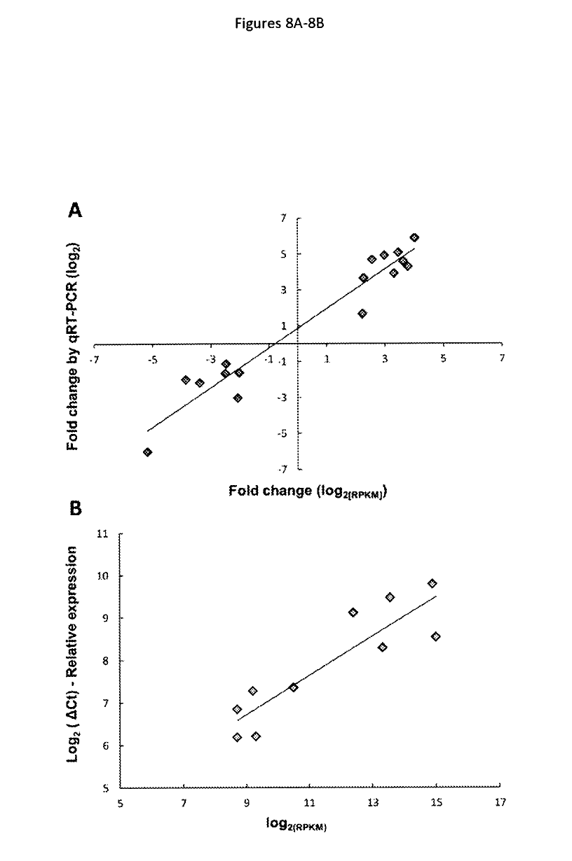

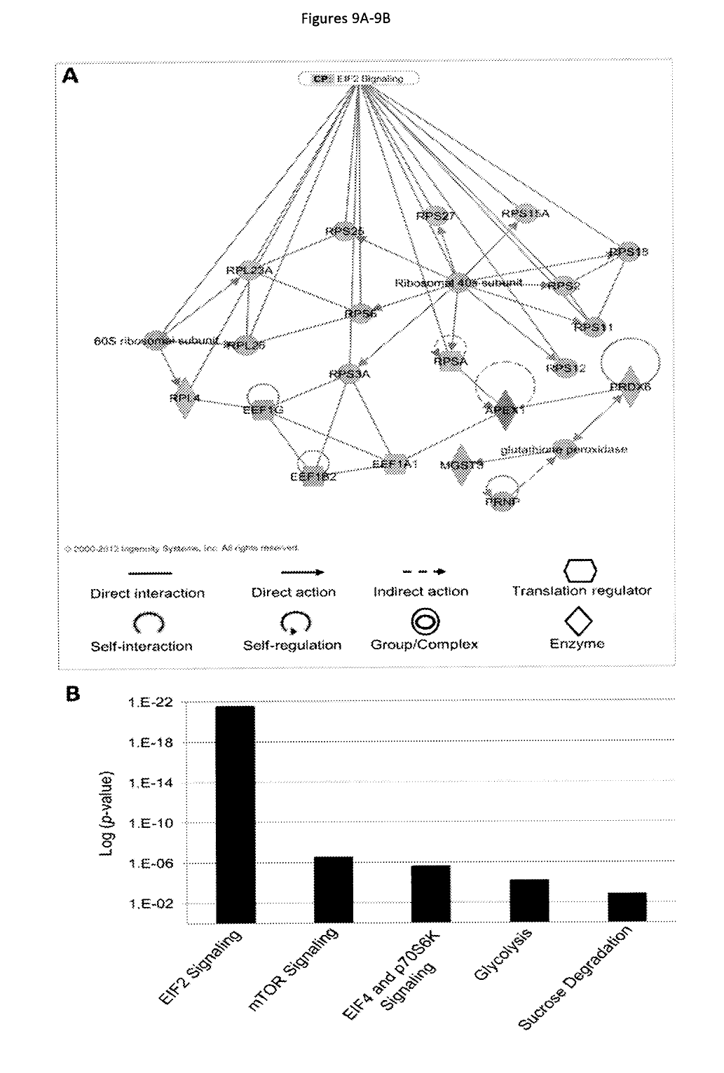

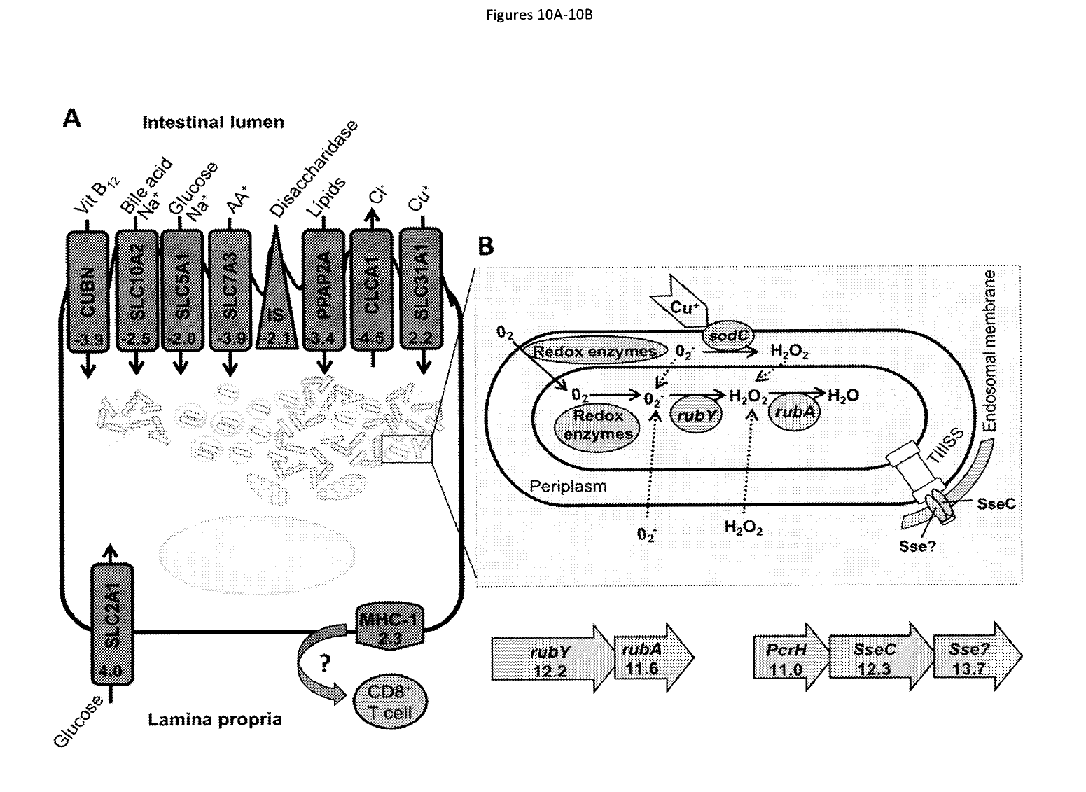

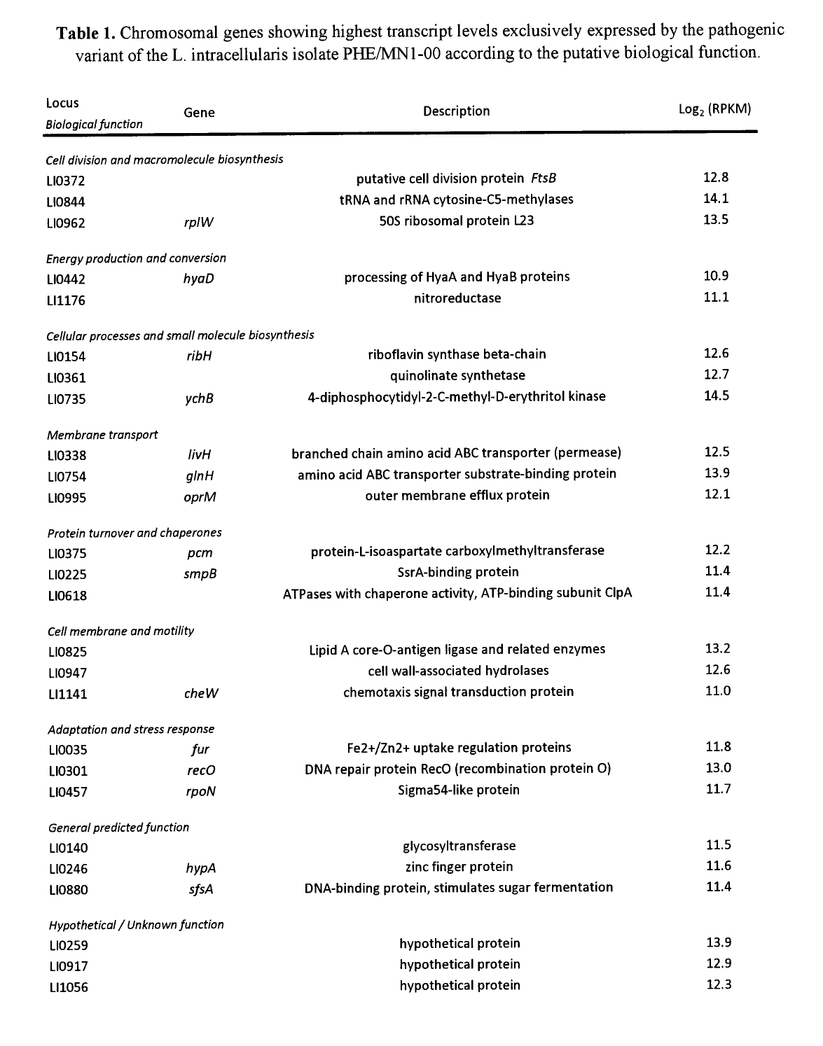

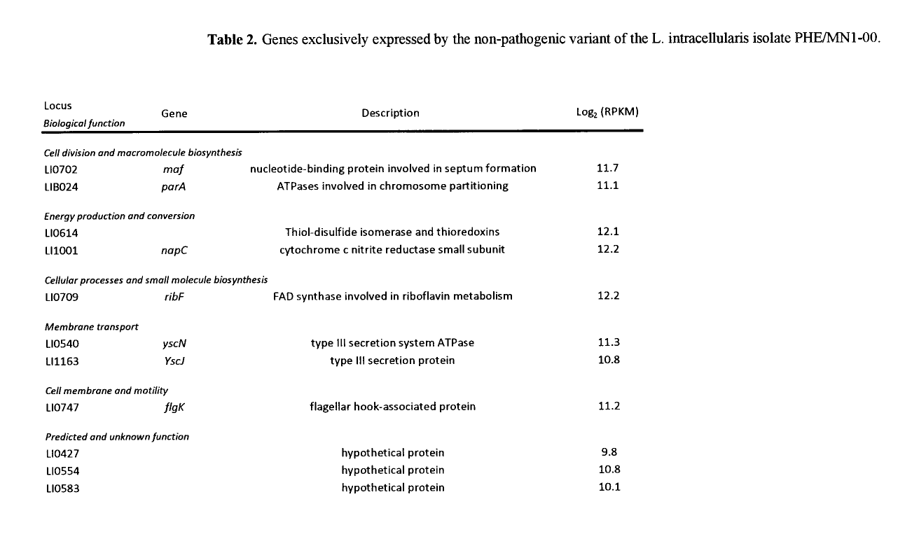

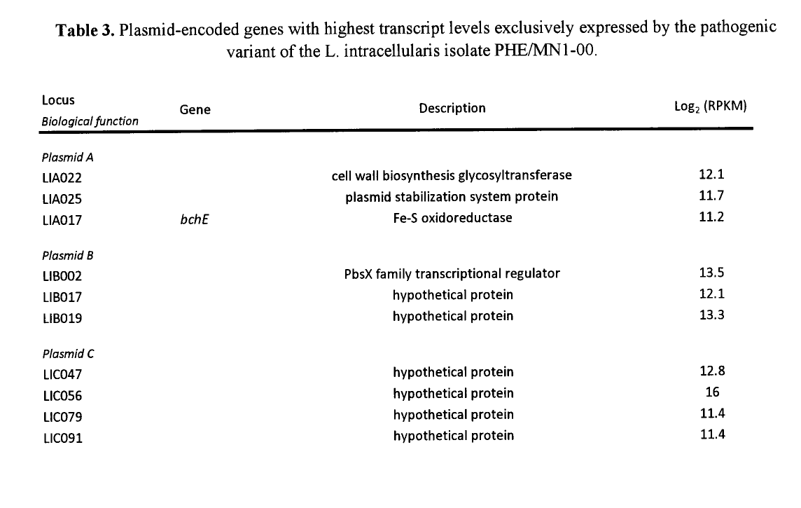

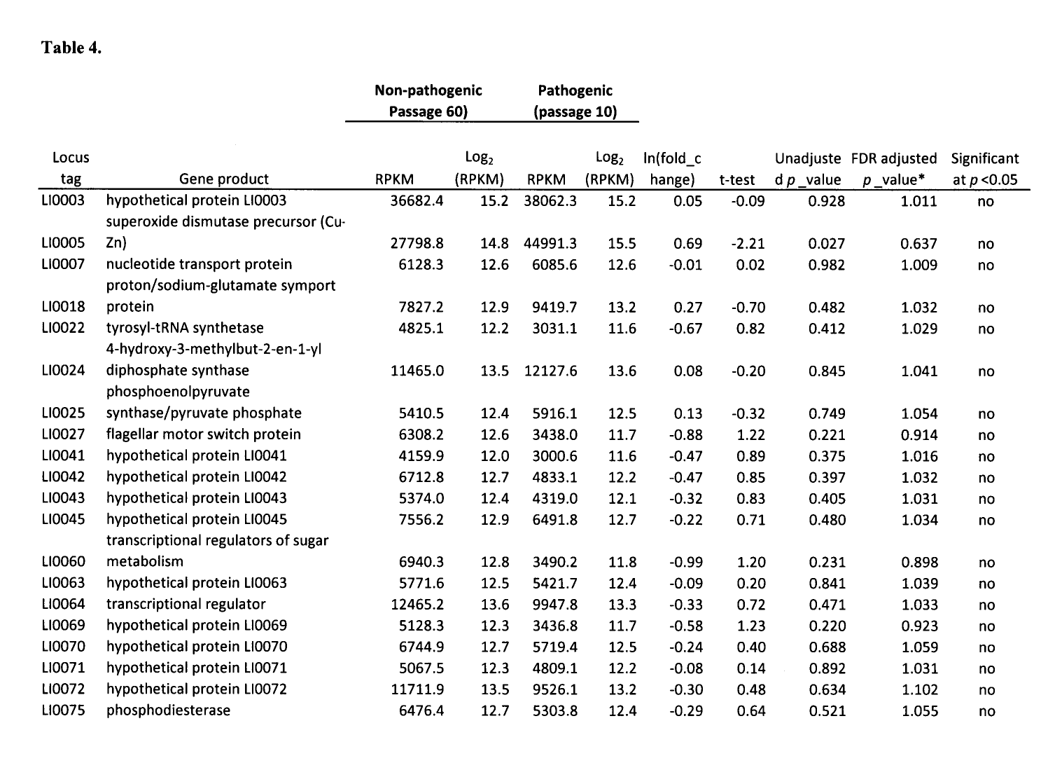

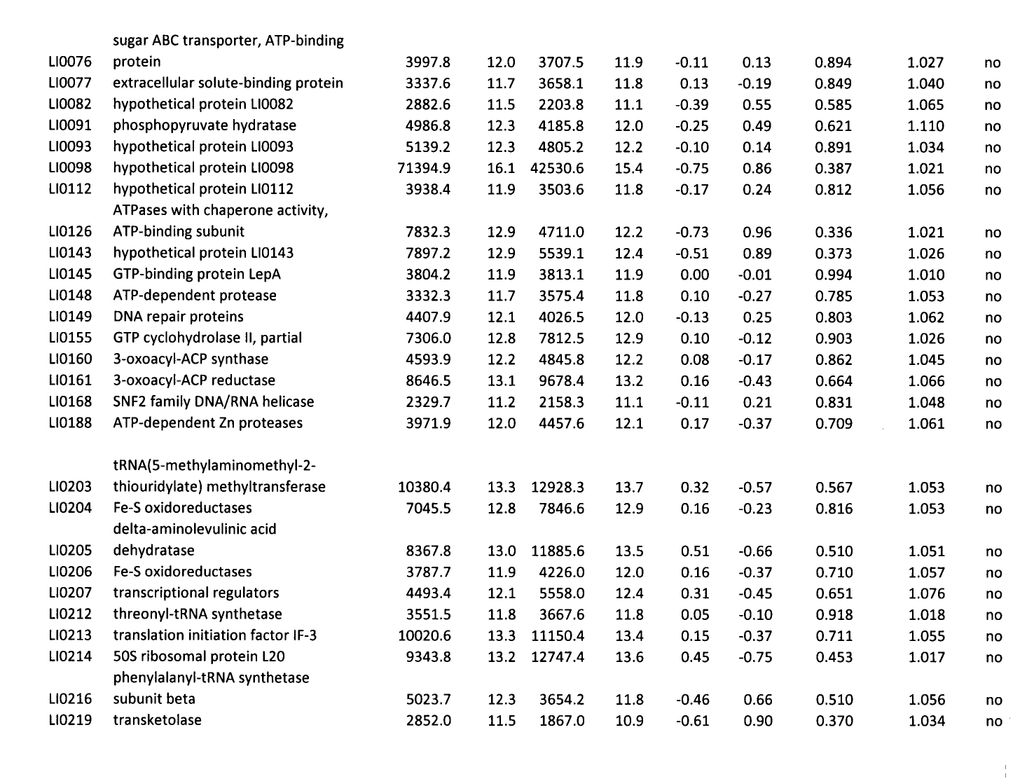

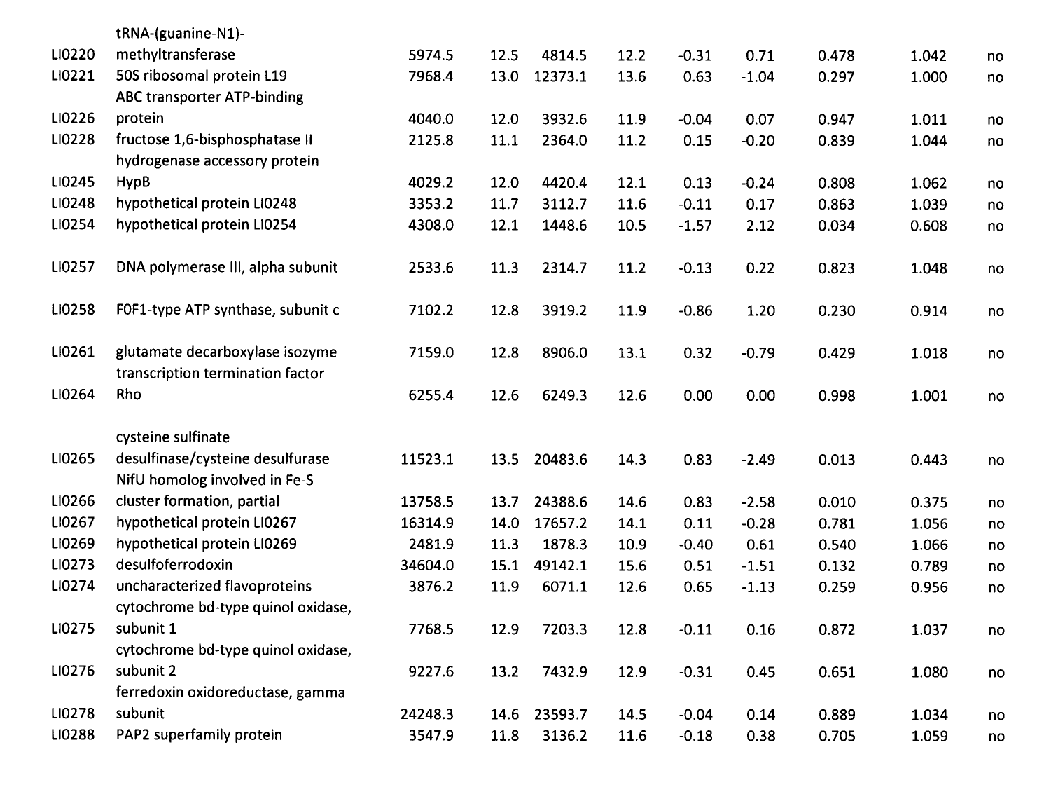

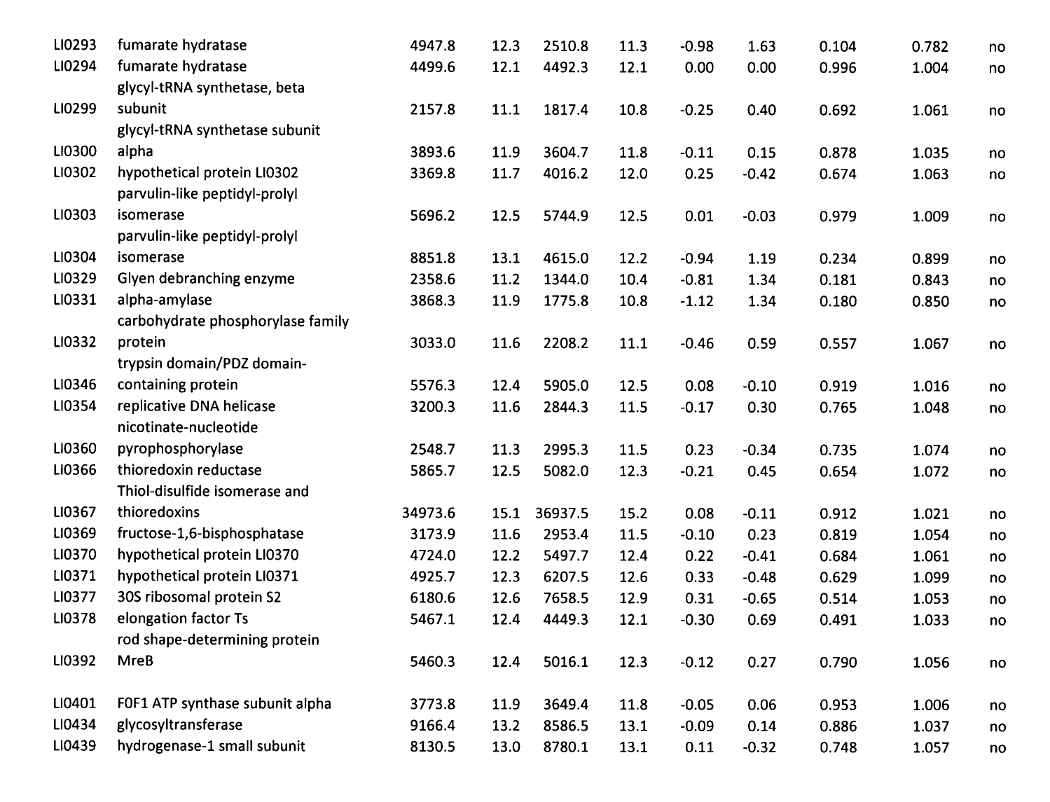

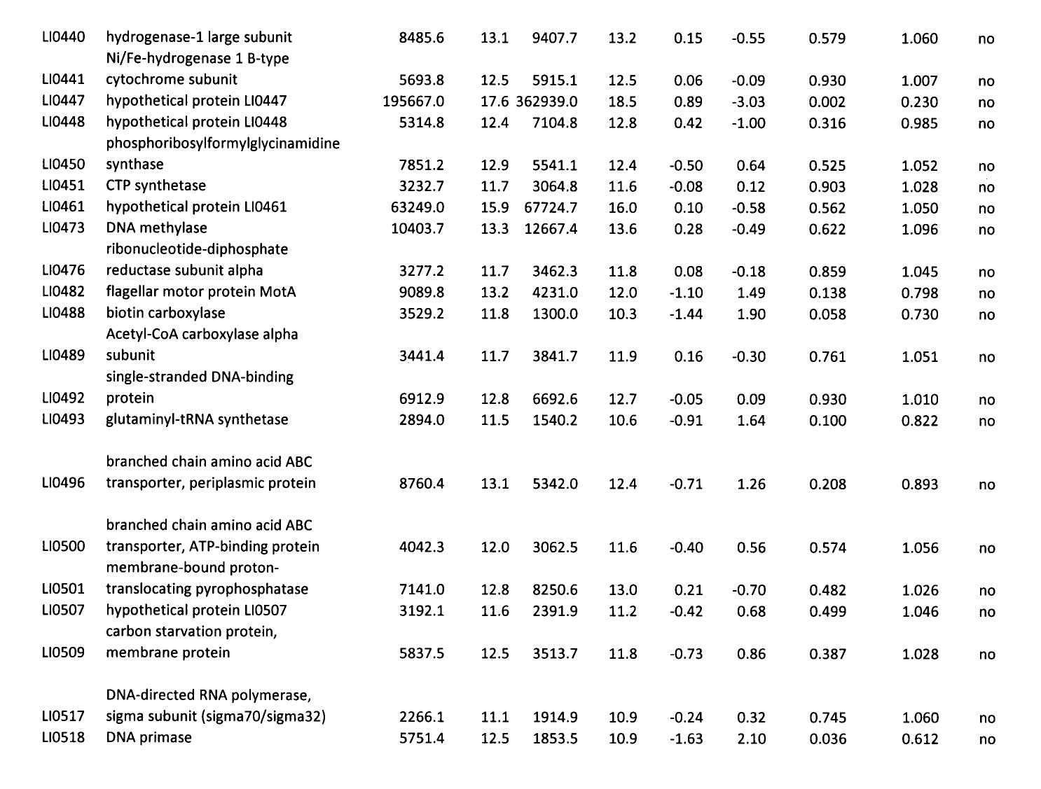

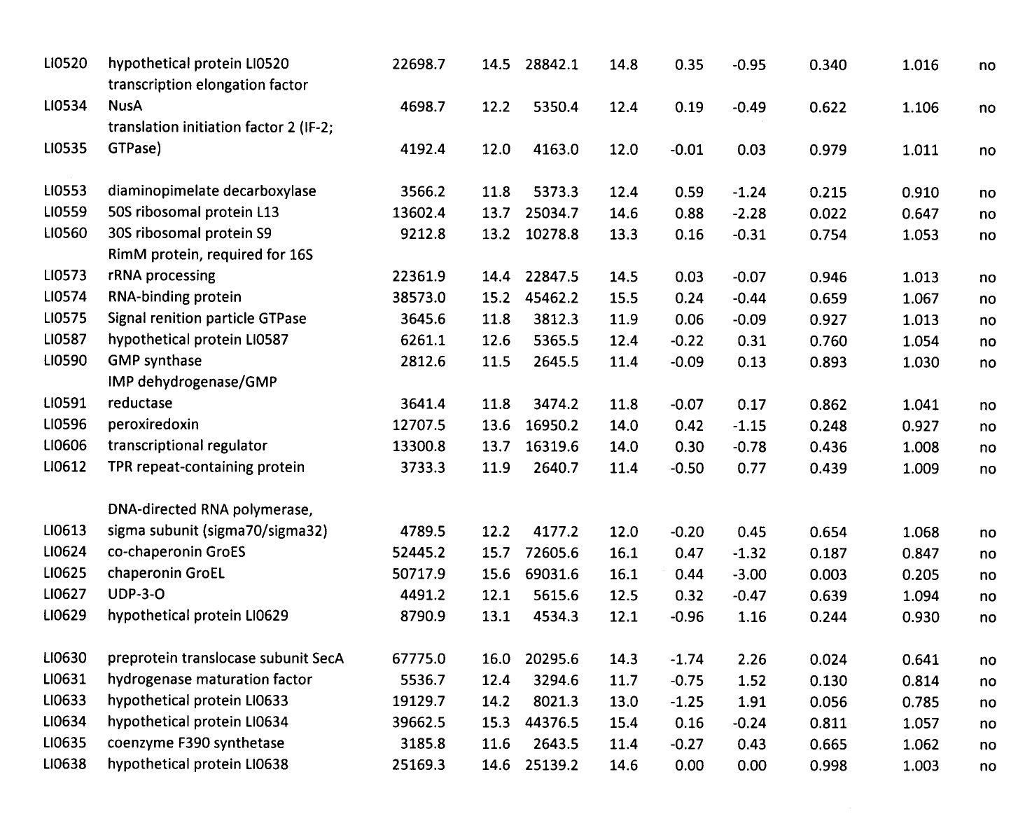

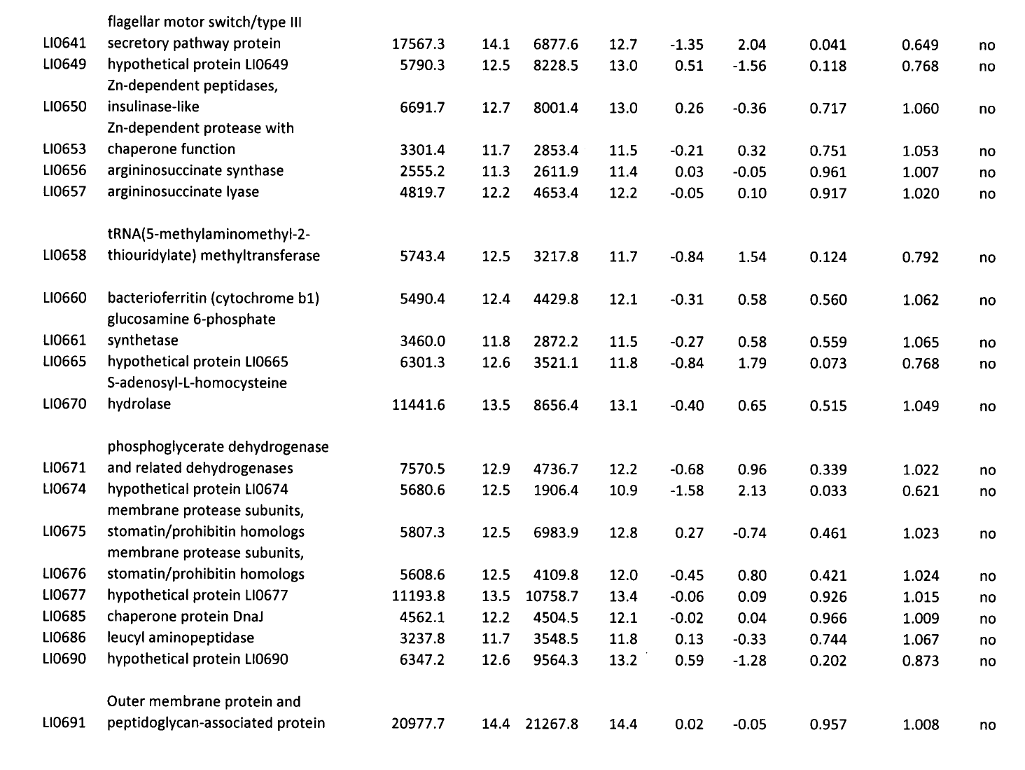

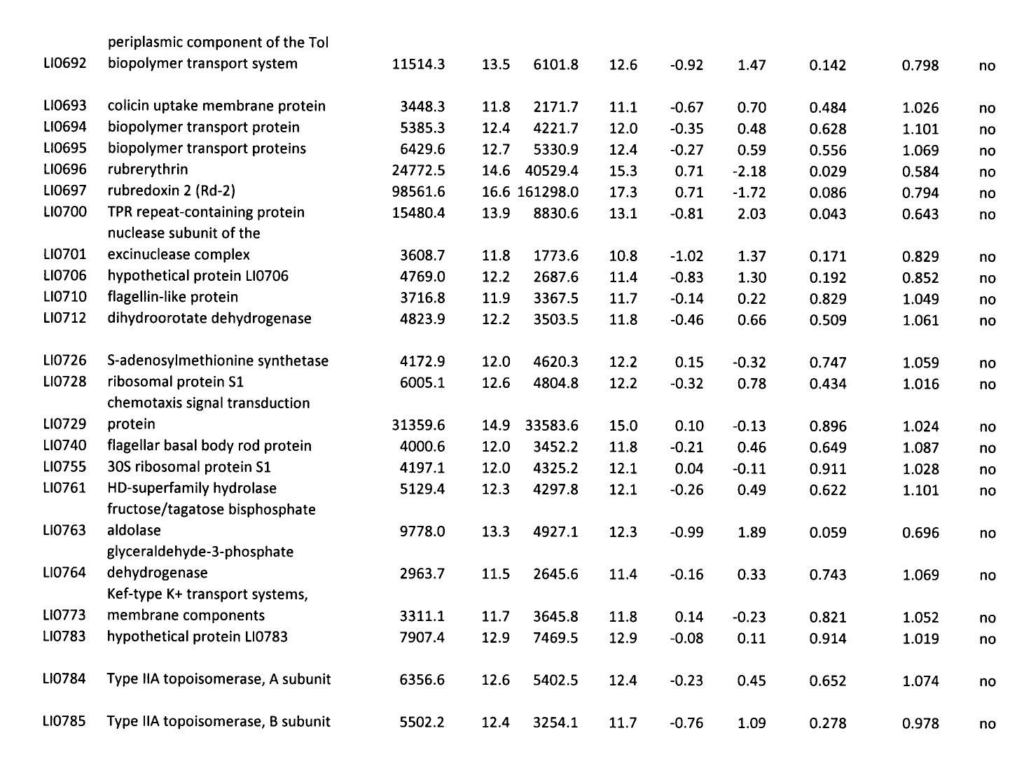

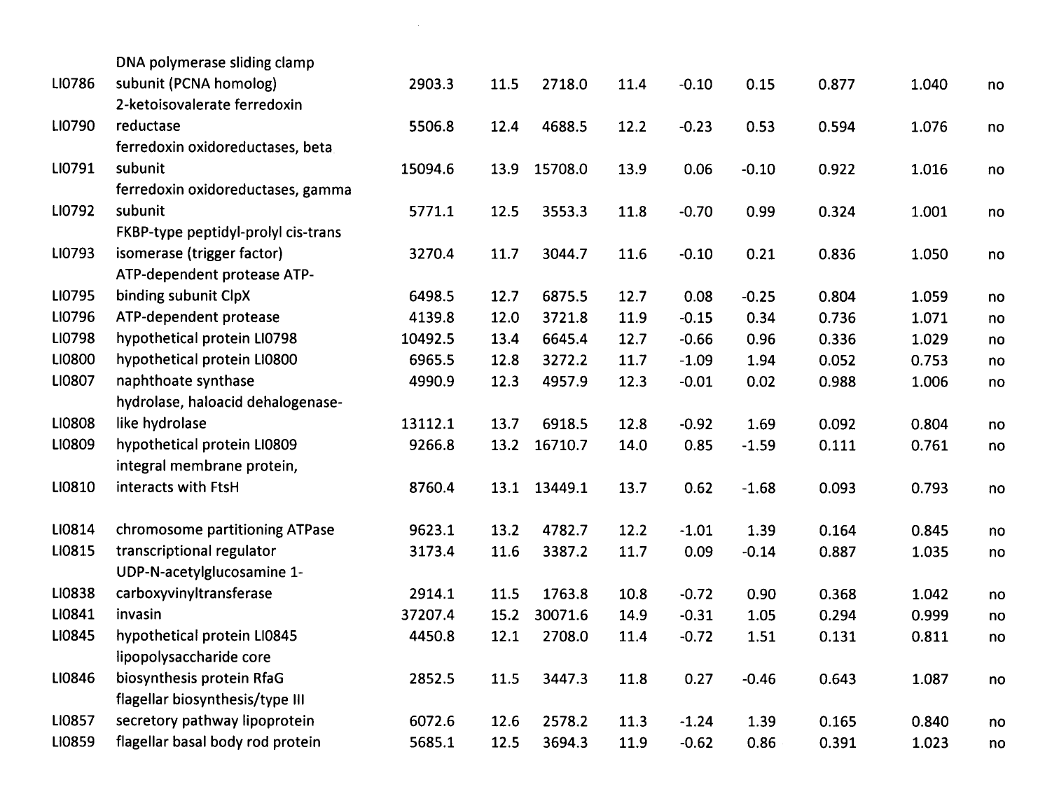

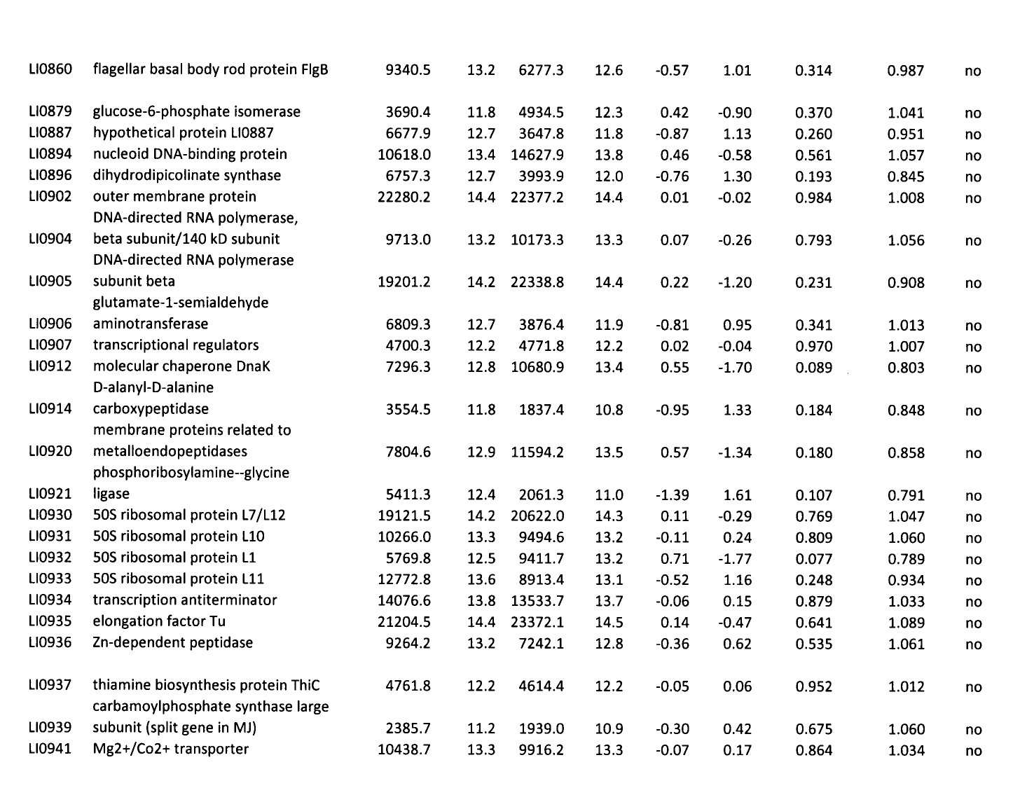

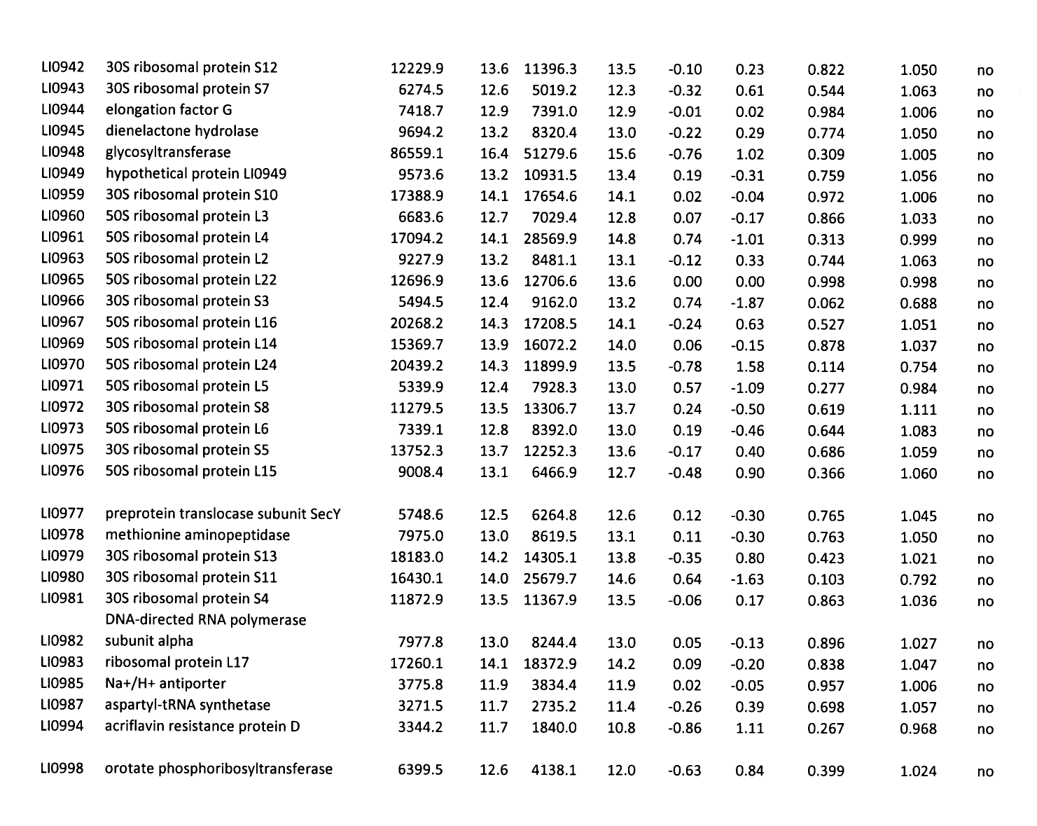

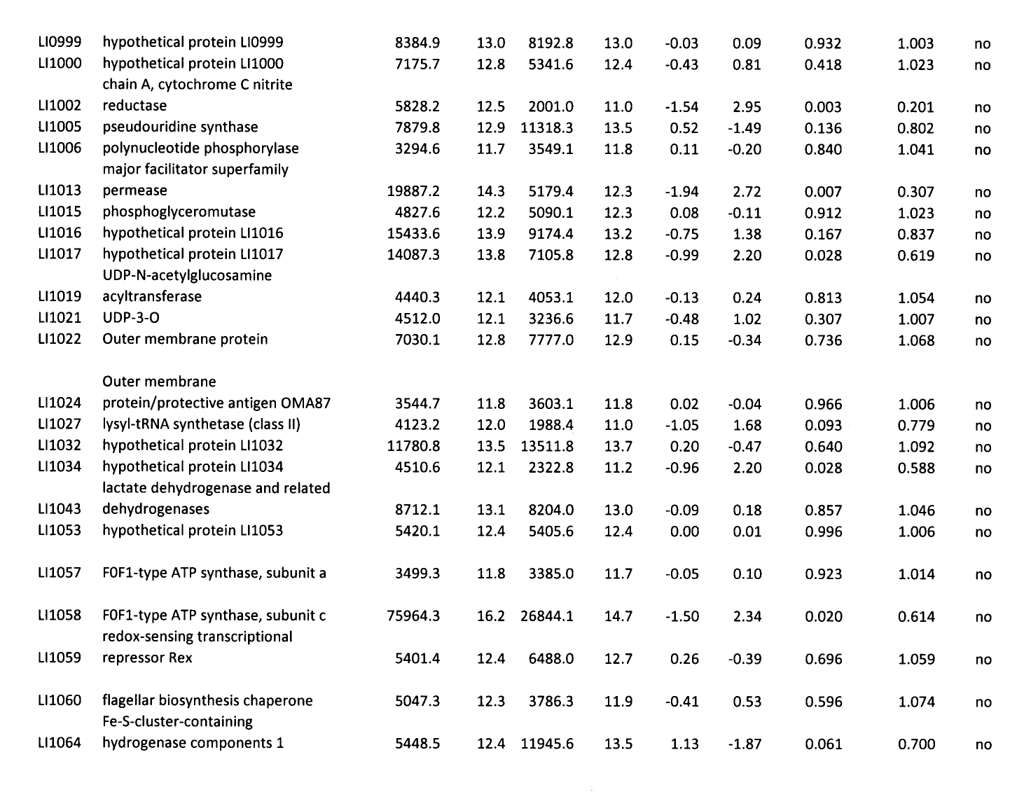

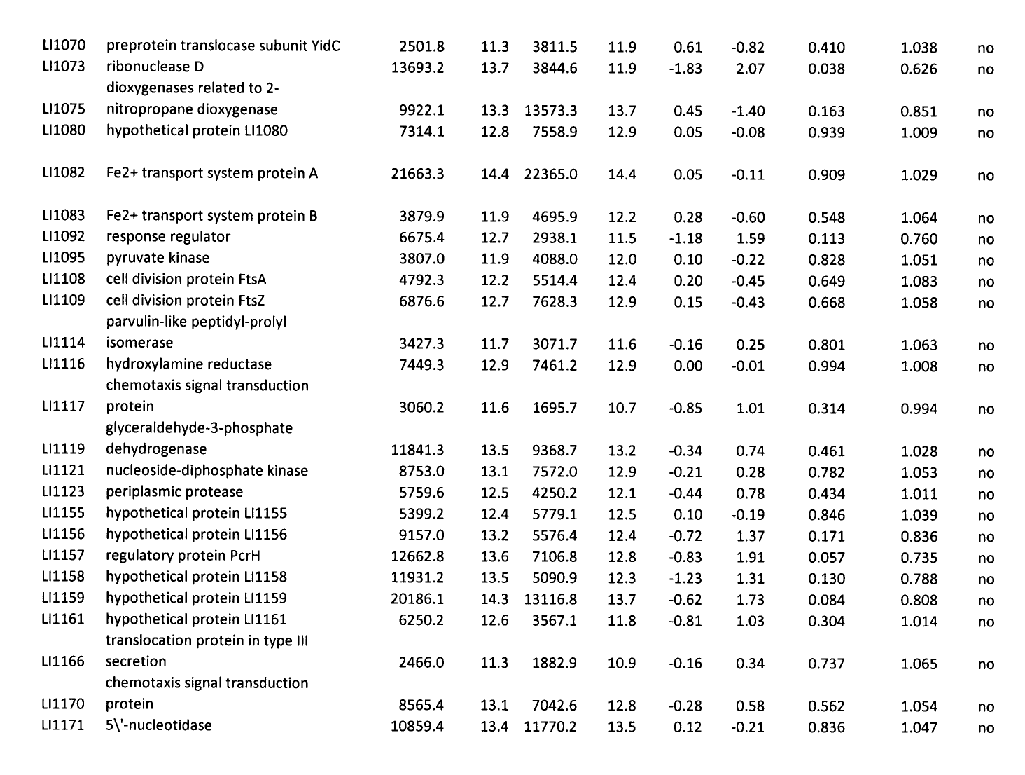

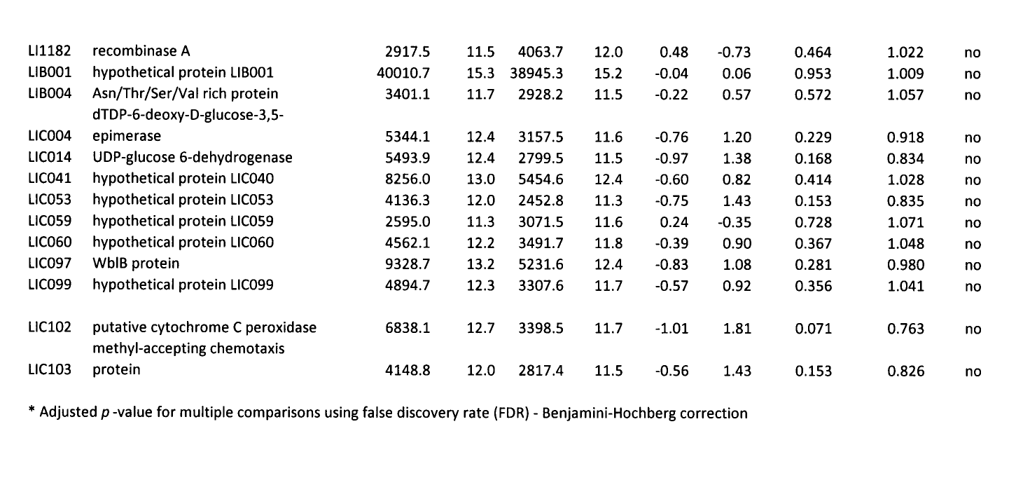

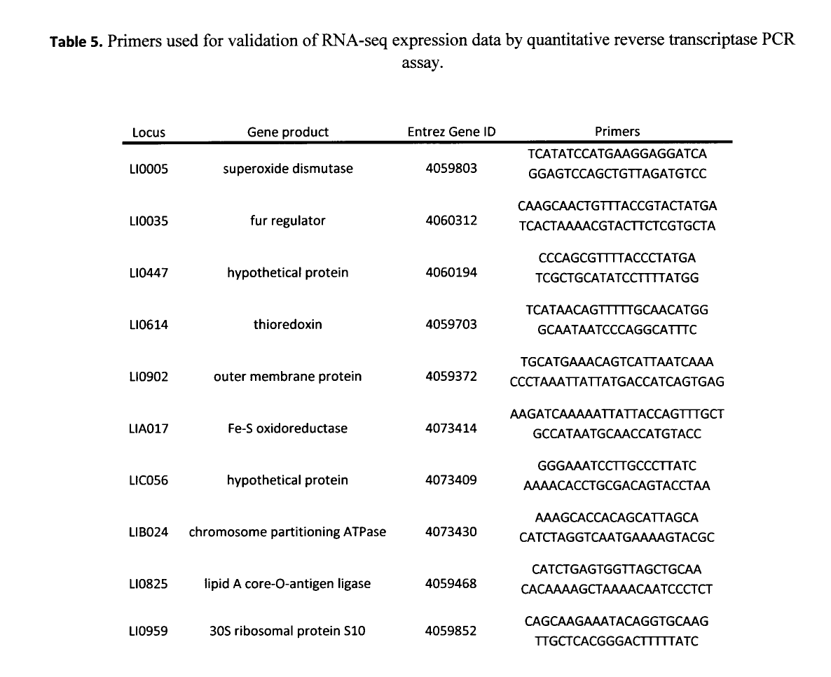

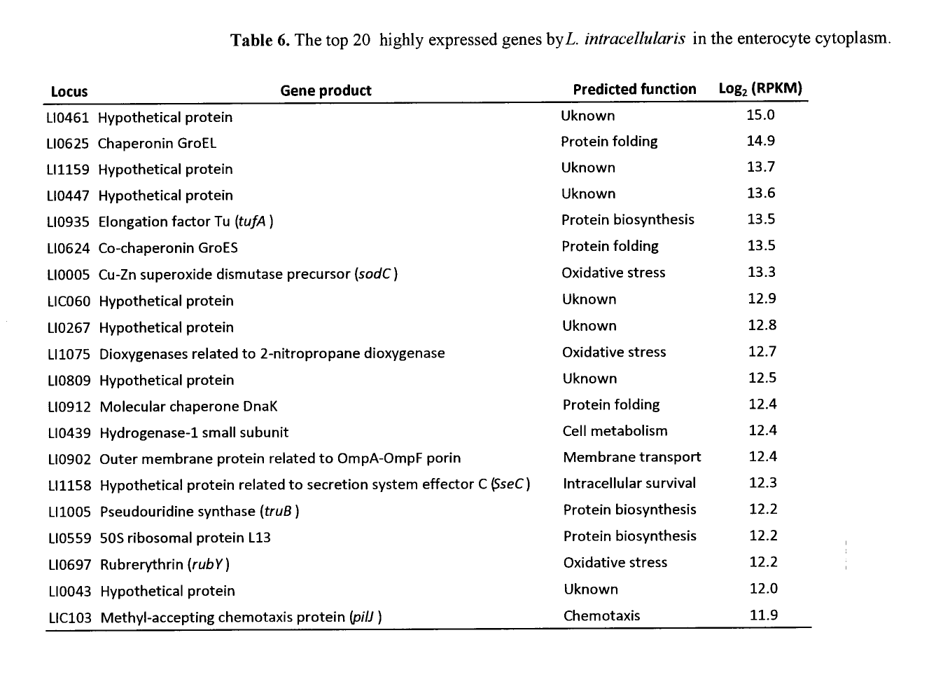

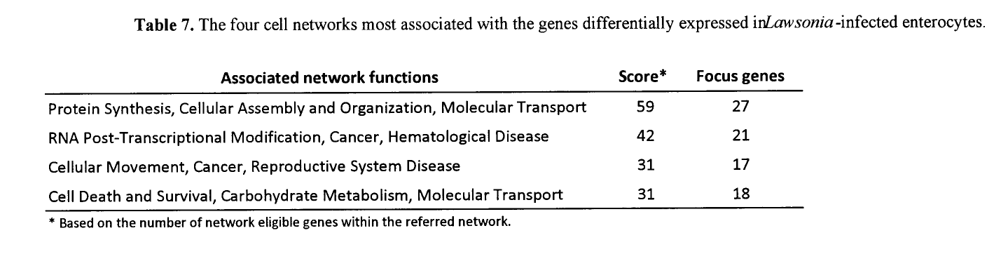

The quality of RNA samples extracted from microdissected tissues was evaluated by assessing bacterial and eukaryotic ribosomal RNAs using Agilent Bioanalyzer 2100 (FIG. 7C). The successful recovery of high quality eukaryotic RNA from microdissected cells using the LCM procedures described in our study has been well-established (Mikulowska-Mennis A, Taylor T B, Vishnu P, et al. High-quality RNA from cells isolated by laser capture microdissection. Biotechniques 2002; 33:176-9). Since studies using prokaryotic RNA from microdissected cells have not been well-explored, the feasibility of using L. intracellularis mRNA for sequencing was confirmed by one-step RT-PCR using specific primers targeting three housekeeping genes (FIG. 7D).

The sequence reads representing the RNA transcripts derived from the host cells and the intracellular bacteria were mapped onto both pig (S. scrofa 10.2) and the L. intracellularis (PHE/MN1-00) reference genomes available at NCBI. From a total average of 22,136,064 reads generated, 82% (18,280,138) and 4% (1,000,440) were mapped against the porcine and the bacterium genome, respectively (Table 10). A total of 11,778 genes were expressed in the porcine transcriptome and met the criteria for differential expression analysis. Up-regulation of 119 and down-regulation of 46 protein coding genes were identified in infected enterocytes (fold-change≧2.0; p<0.05) (Table 11).

In the bacterial transcriptome analysis, 754 protein coding genes had at least one mapped read against the reference DNA sequence of the L. intracellularis PHE/MN1-00 isolate, which was the same isolate used in the experimental infection. The top 20 genes highest expressed by L. intracellularis and their respective normalized transcription levels based on the RPKM values are described in Table 6.

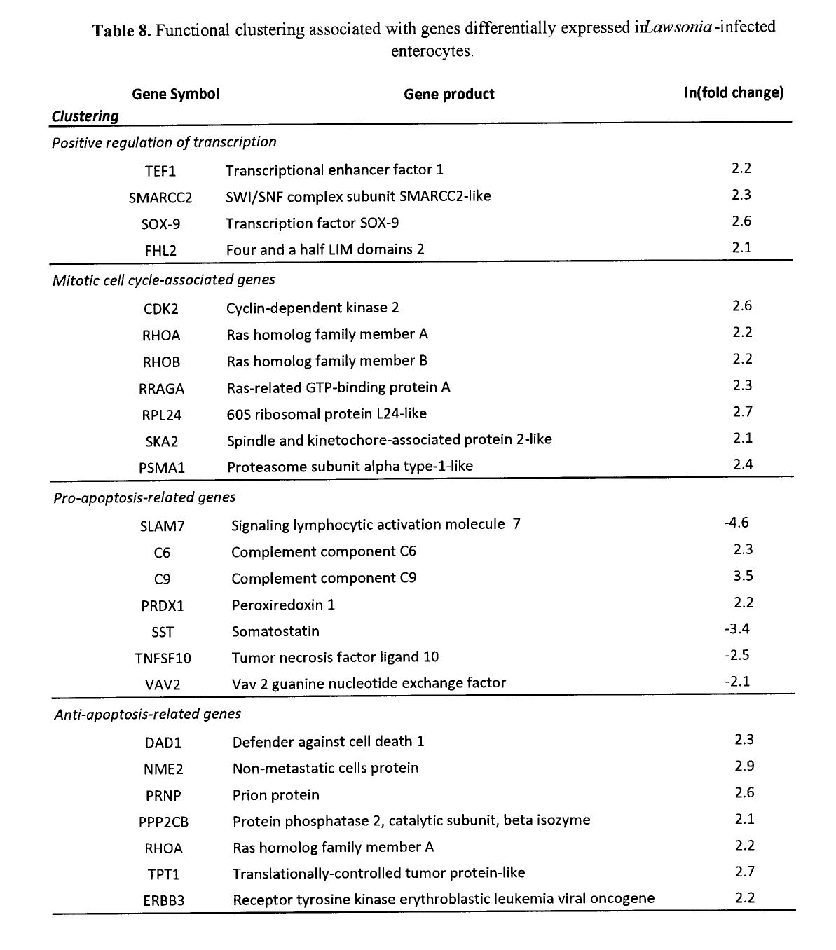

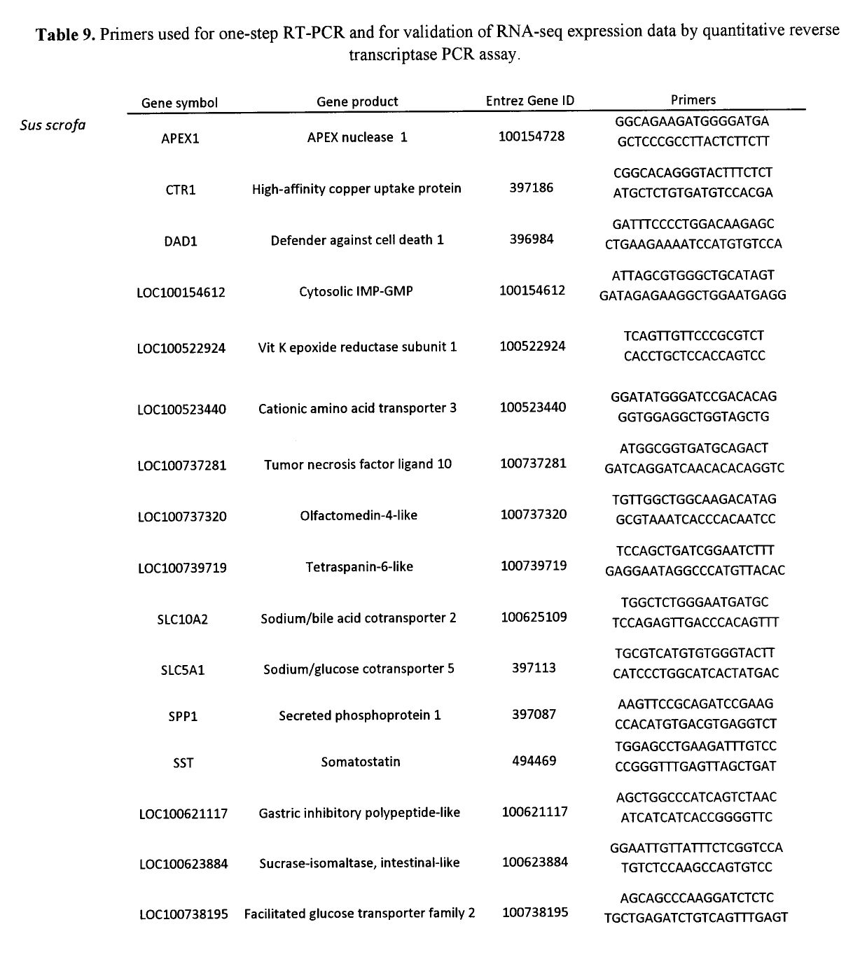

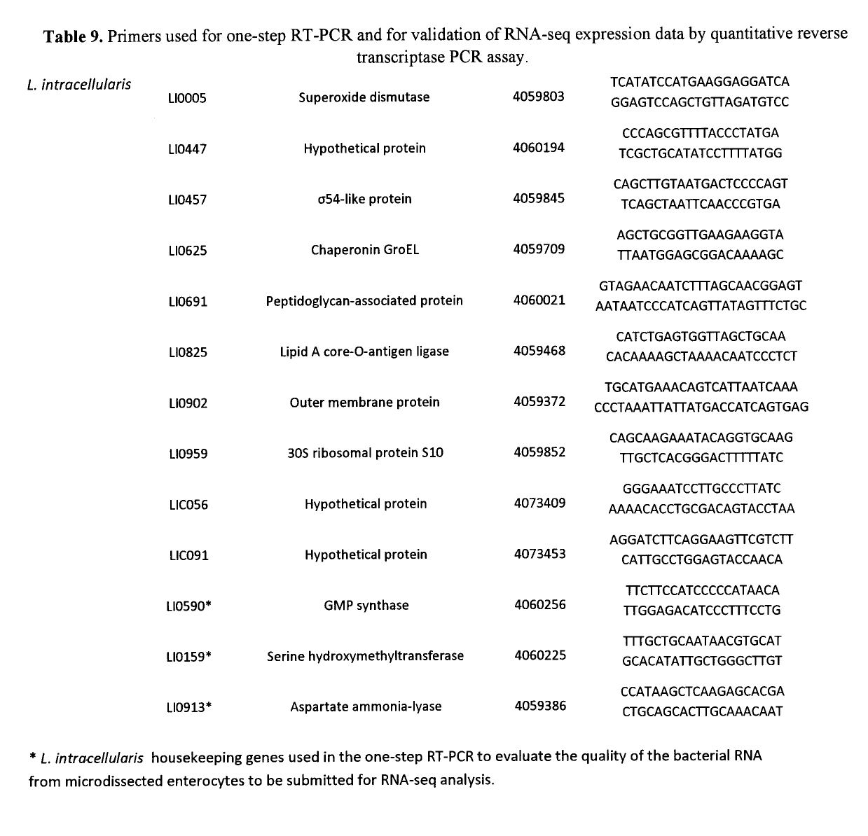

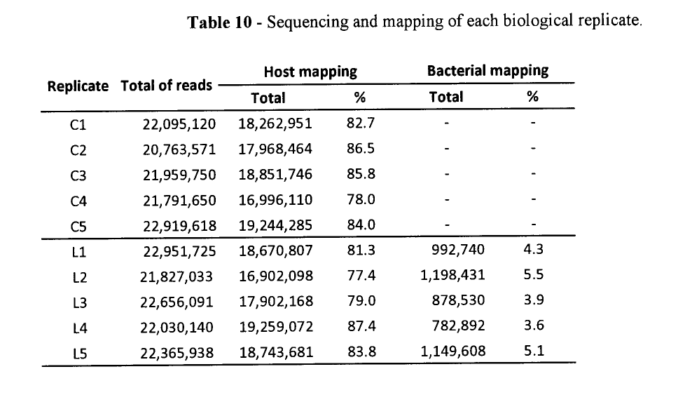

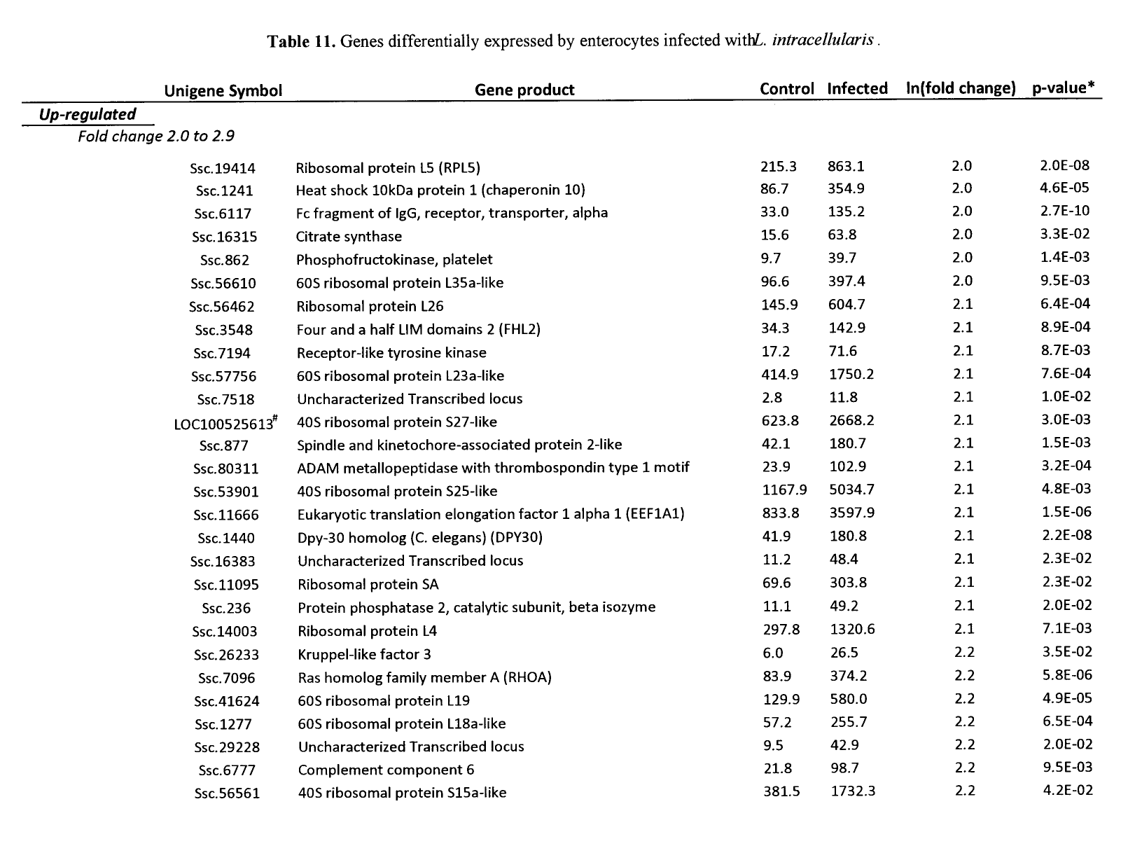

The RNA-seq expression data were validated by qRT-PCR based on the relative quantification of 16 differentially expressed genes from the host and 10 bacterial genes (Table 9). The averages of fold-change in gene expression from infected enterocytes and the relative transcriptional levels of L. intracellularis genes were plotted against the log₂ (RPKM) (FIGS. 8A-8B. The log₂ transformed fold-change from the host gene and transcript levels from the bacterial genes were positively correlated on a linear regression model (p-value<0.05). Based on the qRT-PCR validation, the RNA-seq data properly estimated the fold-change expression in infected enterocytes and the transcription levels of L. intracellularis genes.

Protein Biosynthesis and Transcription

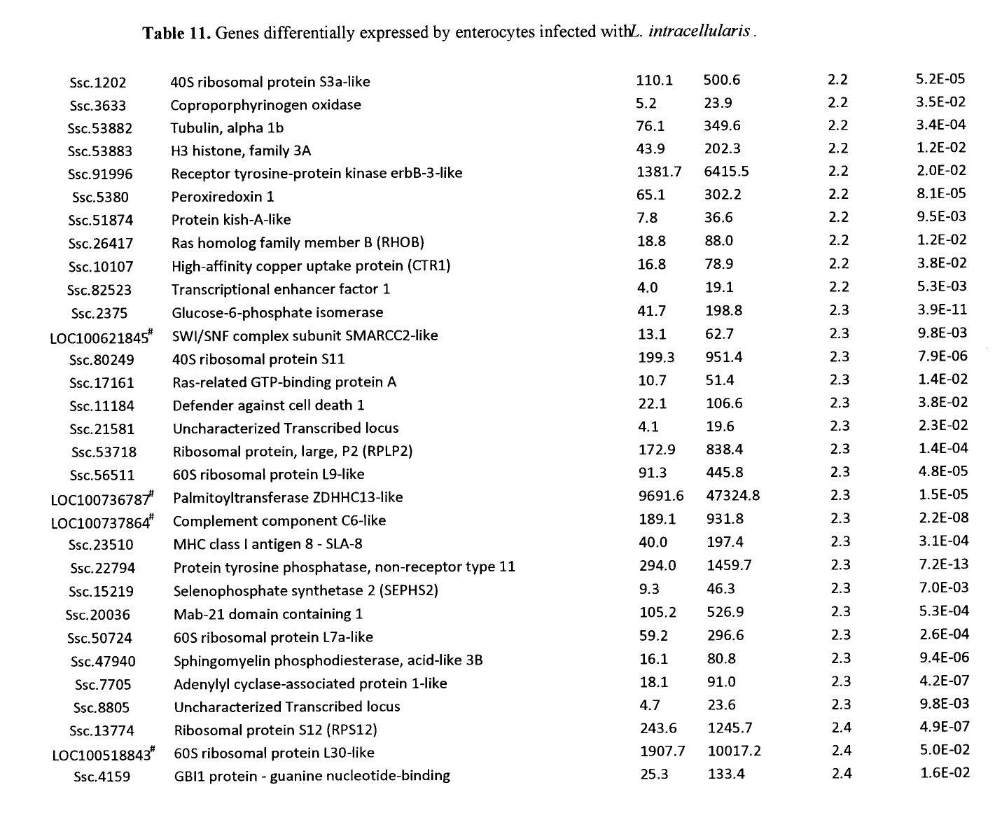

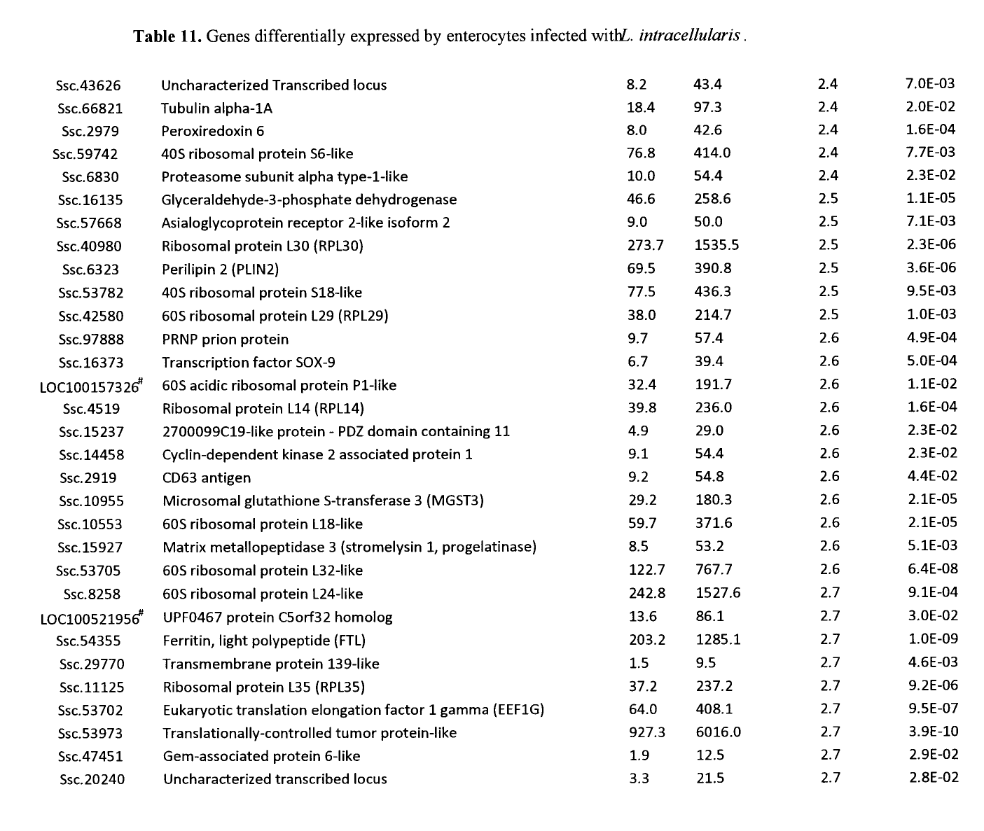

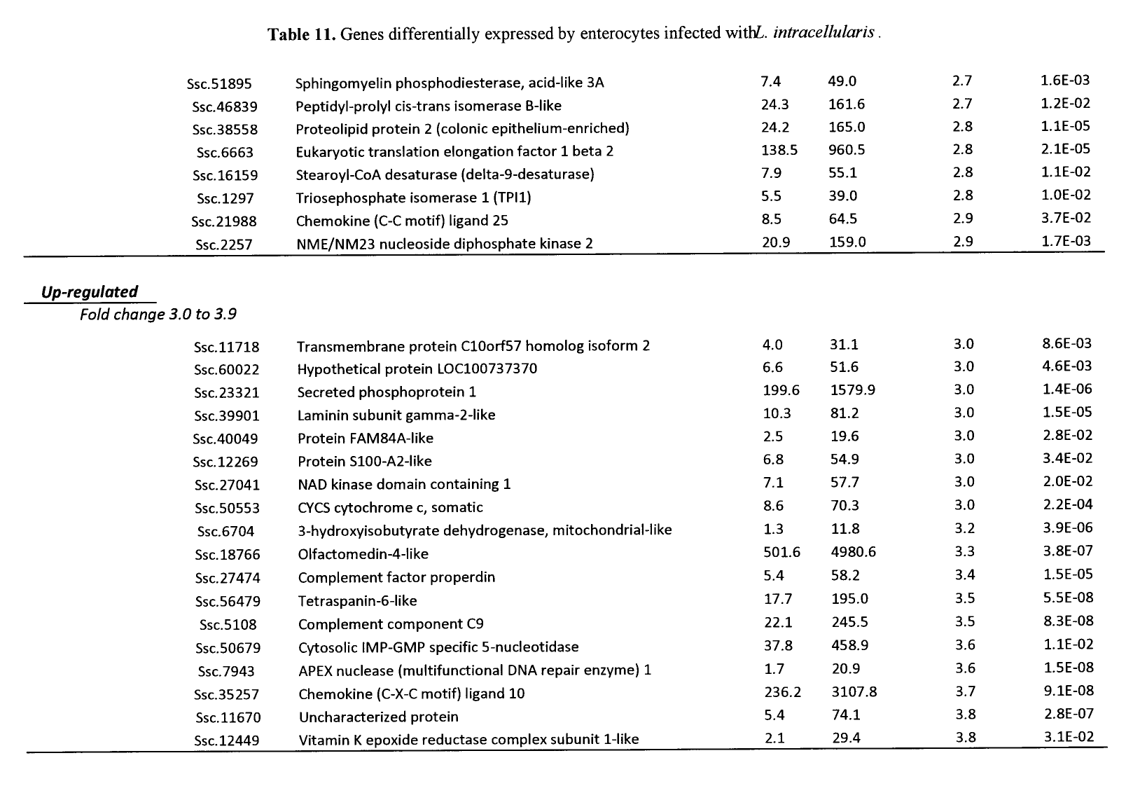

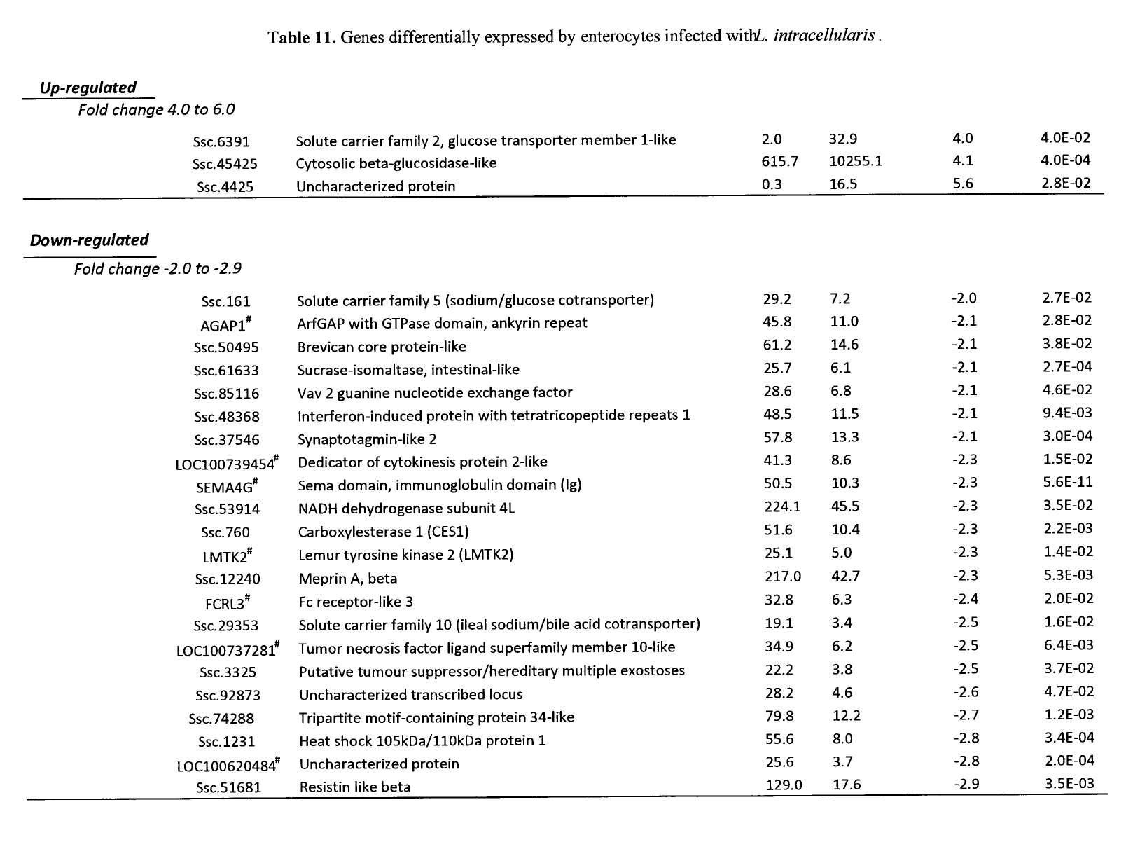

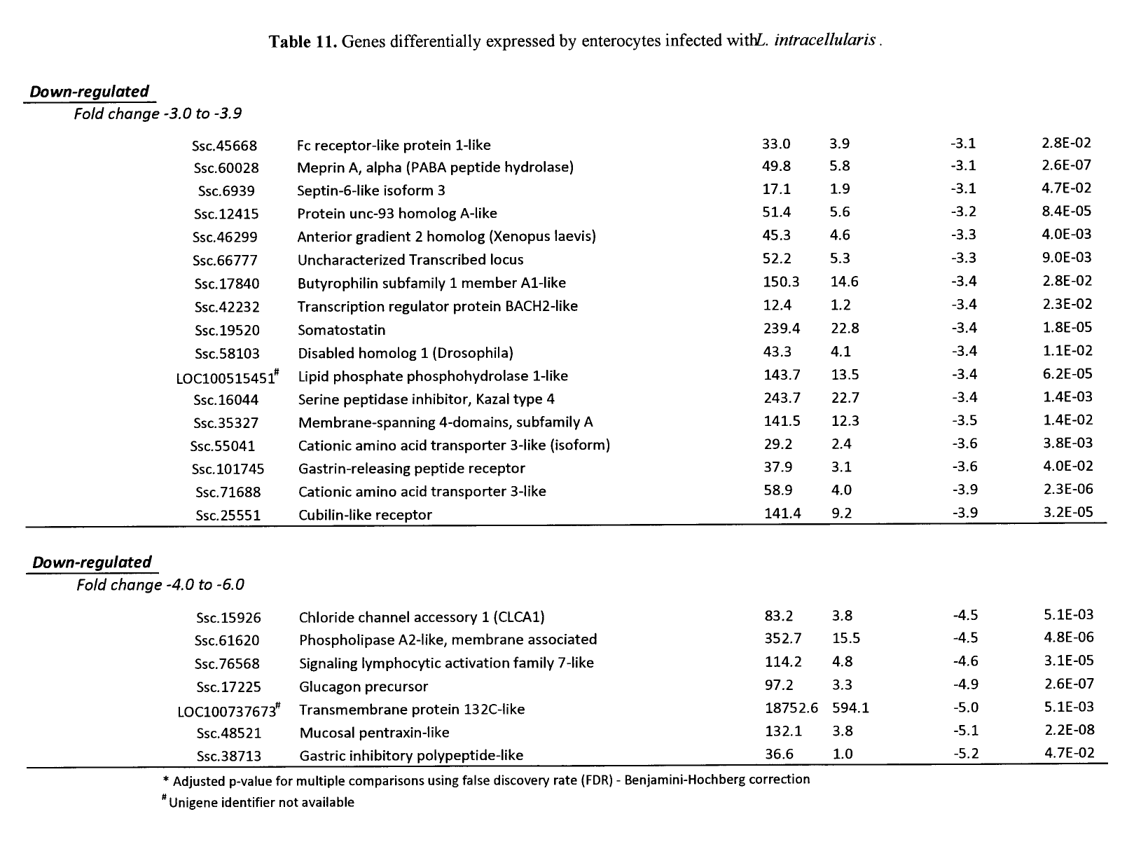

The 165 porcine genes differentially expressed in Lawsonia-infected enterocytes were analyzed regarding their biological functions and molecular networks based on the mammalian gene expression information available in the Ingenuity® System. The system knowledge database recognized and analyzed 144 differentially expressed genes. The IPA system associates the set of differentially expressed genes with cellular networks (focus genes) and creates a score based on the number of network eligible genes they contain (Table 7). The protein biosynthesis network was most correlated with the set of genes differentially expressed and up-regulated in Lawsonia-infected enterocytes. FIG. 9A shows the molecular interaction of this network which mainly includes ribosomal proteins and mRNA translation factors. The eukaryotic initiation factor (IEF2) signaling was the canonical cell pathway (CP) more significantly associated (p-value 2.76E-22) with the genes differentially expressed (FIG. 9B). A remarkable influence of IEF2 signaling on the protein biosynthesis was identified by merging both network and pathway analysis (FIG. 9A).

Functional clustering analysis using DAVID knowledge database confirmed the involvement of the 27 differentially expressed genes in the protein biosynthesis (data not shown) and also showed the association of four genes acting in the positive regulation of transcription (Table 8). The activation of these two cellular processes (transcription and protein biosynthesis) revealed a global increasing in the cell metabolism in response to the L. intracellularis infection and it has also been described to occur during the gap phase 1 (G₁) of the host cell cycle.

Cell Cycle and Apoptosis

In addition to the positive regulation of transcription, DAVID clustering analysis identified genes associated with cell cycle and apoptotic events differentially expressed in infected enterocytes (Table 8). Cell cycle-associated genes mainly represented by Ras homolog proteins were significantly induced in association with Cyclin-dependent kinase 2 (CDK2). The aberrant activation of Rho-genes including those described in our study has been well-described in oncogenesis by causing deregulation of cell cycle progression and promoting cell proliferation. Additionally, the co-expression of Rho proteins and Cyclin-dependent kinases (CDKs) specifically act by stimulating the entry and progression of the G₁ phase of the host cell cycle.

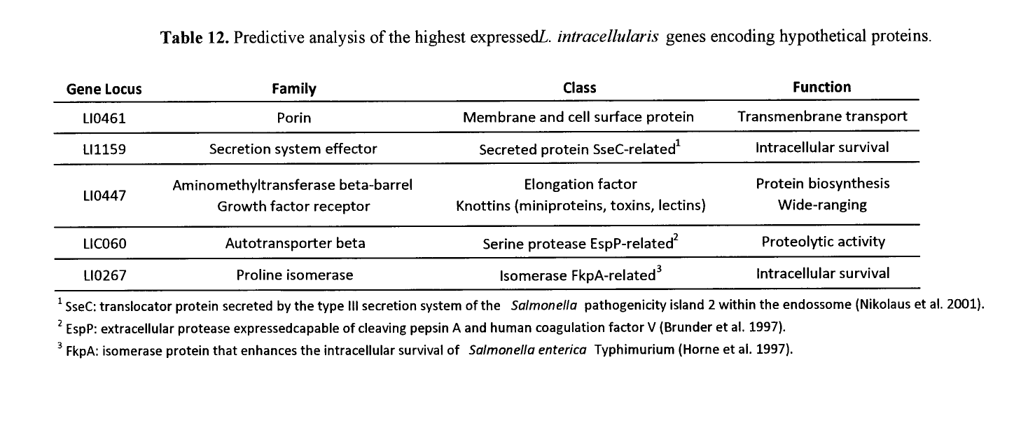

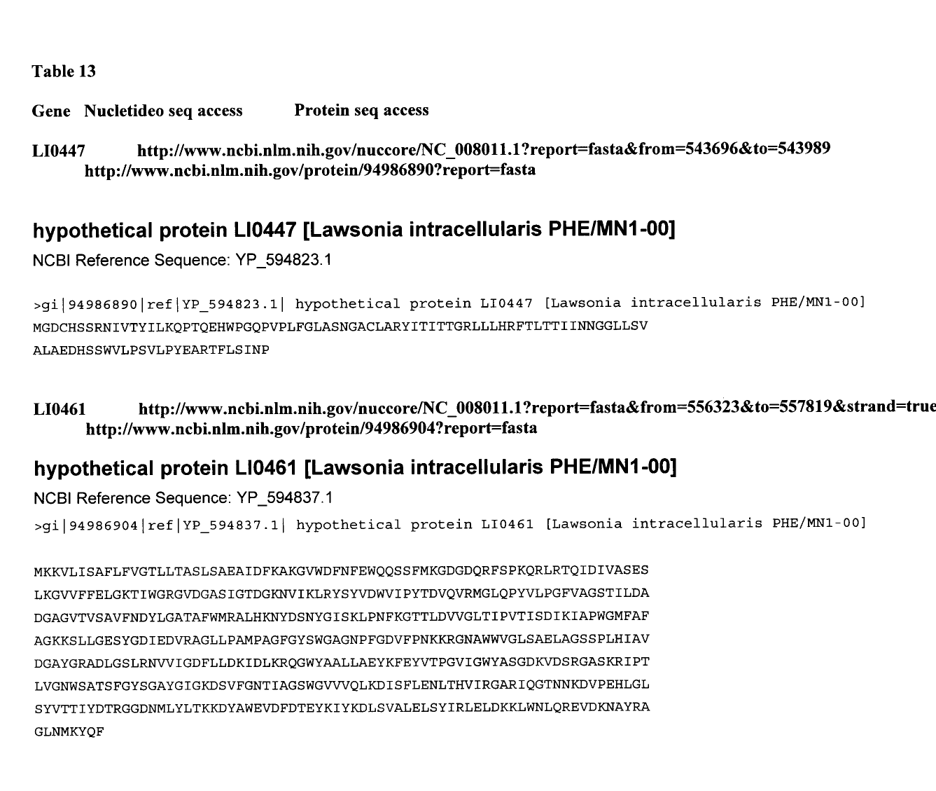

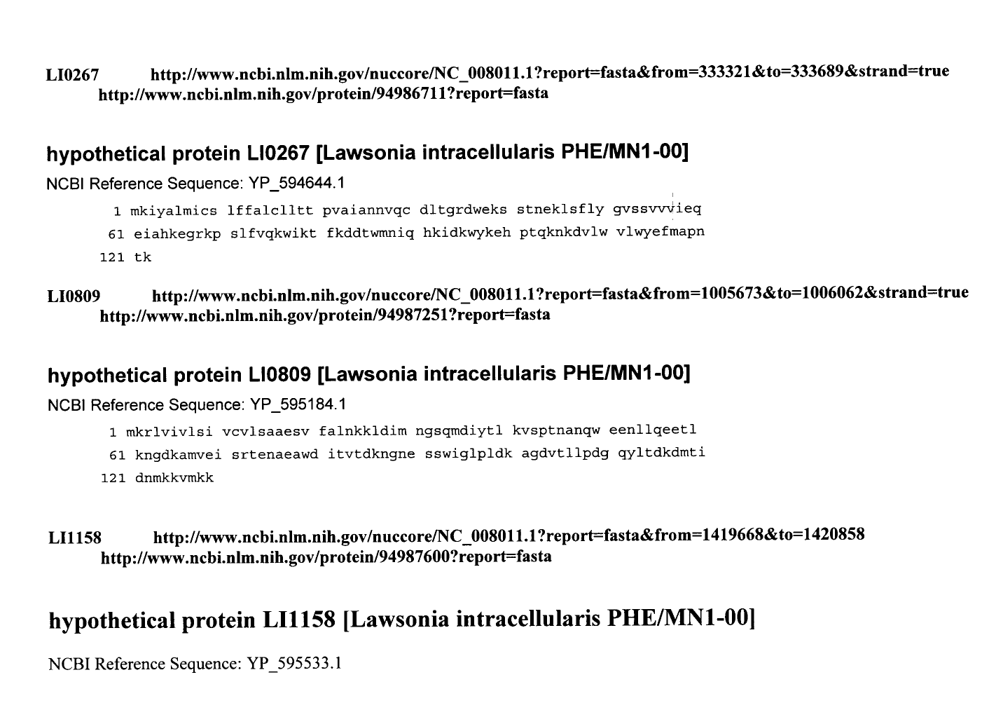

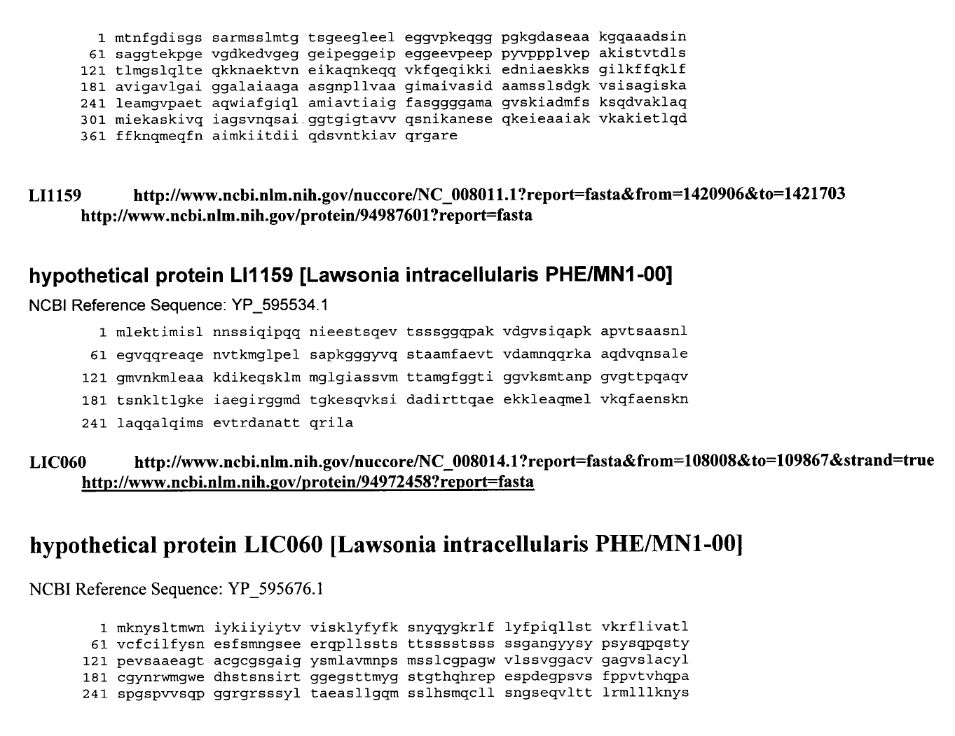

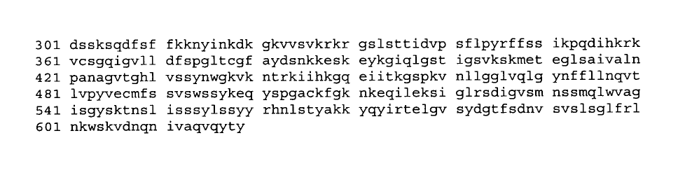

The functional clustering analysis revealed 14 differentially expressed genes associated with pro- and anti-apoptotic events (Table 8). All seven genes related to anti-apoptotic events were up-regulated in infected cells. Among the pro-apoptotic genes, four were down-regulated and three up-regulated. Interestingly, two of these pro-apoptotic genes that are also involved in the cellular immune response against intracellular pathogens (Signaling lymphocytic activation molecule 7 and Tumor necrosis factor ligand 10) were down-regulated. On the other hand, significant up-regulation of genes encoding the major histocompatibility complex class I (MHC-I) was identified in Lawsonia-infected enterocytes (FIG. 10A).

Nutrient Acquisition and Electrolyte Secretion

Consistent down-regulation of numerous genes expressed in the apical membrane of enterocytes that are involved in nutrient acquisition was observed in Lawsonia-infected cells (FIG. 10A). These membrane transporters are involved in the absorption of carbohydrates (sodium/glucose co-transporter and sucrase-isomaltase), amino acids (cationic amino acid transporter), bile acid (sodium/bile acid co-transporter), lipids (lipid phosphate phosphohydrolase) and Vitamin B₁₂ (cubilin receptor). Additionally, the intracellular infection also affects the electrolyte secretion by decreasing expression of the chloride channel gene (CLCA1). The reduction of both nutrient acquisition and electrolyte secretion indicates that L. intracellularis may be able to prevent cell differentiation in immature enterocytes.

In contrast to the down-regulation of genes expressed on the apical membrane, the gene encoding the glucose transporter 1 (also known as solute carrier family 2, glucose transporter member 1) was highly up-regulated in infected enterocytes. This transporter was the third highest expressed gene (Table 9) and its expression also has been reported on the basolateral membrane of human enterocytes in vitro and rat jejunum in vivo. Additionally, significant up-regulation of the high-affinity copper uptake protein (CTR1) involved in copper absorption was also found in infected enterocytes (FIG. 10A).

Bacterial Interactome

The transcriptional landscape of the intracellular bacteria was determined by classifying all genes with at least one mapped read into one of three levels of expression: low, moderate and high. As expected, genes encoding ribosomal-related proteins were the functional category most associated with the mapped reads and the majority of these genes exhibited moderate to high expression. The L. intracellularis interactome was characterized and discussed based on the most highly expressed bacterial genes (Table 6).

The functional categories of bacterial genes that were highly expressed included those involved in protein folding (e.g. groES-groEL operon and chaperone dnaK) and biosynthesis (e.g. elongation factor Tu), oxidative stress (e.g. Cu—Zn superoxide dismutase, rubY-rubA operon and dioxygenases), secretion system effector-related proteins (PcrH-SseC-LI1159 operon) and various hypothetical proteins. Since we were not able to distinguish free bacteria in the cell cytoplasm from those organisms within cell endosomes, the interactome was built and discussed considering both scenarios. FIG. 10B illustrates the biological activities of the bacterial proteins encoded by highly expressed genes considering the intracellular microenvironment of Lawsonia-infected enterocytes.

While redox enzymes catalyze reduction of O₂ derived from the cell cytoplasm, Cu—Zn superoxide dismutase C (sodC) and rubrerythrin-rubredoxin operon (rubY-rub) neutralize reactive oxygen species (O₂⁻ and H₂O₂) generated in the endosome or from the reduced O₂ molecule (FIG. 10B). An operon genetically related to the Salmonella pathogenicity island 2 (SPI2) composed of three genes, a chaperone (PcrH), an effector protein (SseC) and a hypothetical protein LI1159 (referred as Sse?) was highly expressed (FIG. 10B). Interestingly, genes encoding the type III secretion system (TIIISS) apparatus whereby these effector proteins would be delivered to the cell cytoplasm showed only moderate expression (data not shown) and were located downstream of the SPI2-related operon on the L. intracellularis chromosome.

Among the ten genes most highly expressed by the intracellular bacterium, five represented hypothetical proteins. A summary of the predictive analyses evaluating their structures and biological functions are described in the supplemental material (Table 12). Two different families of proteins were predicted for the gene locus LI0447 (aminomethyltransferase beta-barrel and growth factor receptor domain). Additionally, transmembrane proteins (porin and autotransporter), proline isomerase, extracellular protease and secretory factor were also identified based on predictive motifs.

Discussion

The present study used LCM technology to microscopically dissect enterocytes from pigs experimentally infected with L. intracellularis and to characterize the cell-specific transcriptional landscape associated and the bacterial interactome using high throughput sequencing (RNA-seq). The results demonstrated the usefulness of coupling LCM and RNA-seq techniques to study the host-pathogen interaction in a specific cell population present in a heterogeneous tissue. The simultaneous evaluation of the gene expression changes in both the host and the pathogen has been recently designated dual RNA-seq (Westermann A J, Gorski S A, Vogel J. Dual RNA-seq of pathogen and host. Nat Rev Microbiol 2012; 10:618-30). The challenge in studying both RNA transcriptomes from a common sample arises mainly because of the extensive variety of the bacterial organisms regarding their genomic compositions (e.g. % CG contents) and the particular characteristics of each infectious process at the cellular level. Since the abundance of eukaryotic RNA is significantly higher compared to the prokaryotic RNA in an infected cell, a sufficient number of bacteria per host cell followed by an unbiased RNA amplification step are crucial for studying the cell-bacteria interactome. In light of this, we established the endpoint of our study (21 days PI), based on the chronological course of the L. intracellularis infection previously described (Guedes R M, Gebhart C J. Comparison of intestinal mucosa homogenate and pure culture of the homologous Lawsonia intracellularis isolate in reproducing proliferative enteropathy in swine. Vet Microbiol 2003; 93:159-66; McOrist S, Roberts L, Jasni S, et al. Developed and resolving lesions in porcine proliferative enteropathy: possible pathogenetic mechanisms. J Comp Pathol 1996; 115:35-45). Confirming these previous observations, our study showed at 21 days PI, numerous bacteria in proliferative enterocytes indicating the active stage of the infectious process. We hypothesized that the disease at this point was approaching its peak when the intracellular bacteria were exhibiting their virulence factors and the host cells were responding to the pathogen at appropriate levels to allow us to evaluate the changes in gene expression. We believe that earlier endpoints would not provide a sufficient amount of RNA to be recovered and later stages of the disease would not represent the logarithmic phase of the bacterial growth. Additionally, the increasing presence of L. intracellularis within the lamina propria along the disease progression would also reduce the number of bacteria in enterocytes.

A previous chronological analysis using microarray technology evaluated the host response of fibroblastic cells in vitro at three time points (24, 48 and 72 hours PI) (Oh Y S, Lee J B, McOrist S. Microarray analysis of differential expression of cell cycle and cell differentiation genes in cells infected with Lawsonia intracellularis. Vet J 2010; 184:340-5). Altered transcription of genes related to cell cycle and cell differentiation were described. However, cellular proliferation which is the main phenotypic characteristic of the L. intracellularis infections in vivo has not been reproduced in in vitro models (Lawson G H, McOrist S, Jasni S, et al. Intracellular bacteria of porcine proliferative enteropathy: cultivation and maintenance in vitro. J Clin Microbiol 1993; 31:1136-42; Vannucci F A, Wattanaphansak S, Gebhart C J. An Alternative Method for Cultivation of Lawsonia intracellularis. J Clin Microbiol 2012; 50:1070-2). Furthermore, the transcriptional response of mesenchymal cells which are not the natural target cells for L. intracellularis needs to be interpreted with caution.

A single time point analysis of the host transcriptome using intestinal tissues from pigs naturally affected with PE was performed. Although this study provided an interesting snapshot of the transcriptional host response, it used field cases of diarrhea where the samples were co-infected with porcine circovirus type 2 which may be a confounding factor in the evaluation of the expression of genes especially related to the immune response. Additionally, the microarray was performed using entire intestinal tissues. As a result, the specific characterization of the transcriptional host response was impaired by either the heterogeneity of the cell population included in the intestinal tissues or the virus infection. By applying the LCM technique to isolate ileal enterocytes in our study, we confirmed the identification of several genes specifically expressed in the intestinal epithelium of the ileum (e.g. ileal sodium/bile acid cotransporter) corroborating with a recent comprehensive study describing the gene expression atlas of the domestic pig (Freeman T C, Ivens A, Baillie J K, et al. A gene expression atlas of the domestic pig. BMC Biol 2012; 10:90).

Gene network and pathway analyses revealed that protein biosynthesis and activation of transcription were the host cellular events most associated with the genes differentially expressed in Lawsonia-infected enterocytes (FIG. 9A). In agreement with these molecular findings, ultrastructural studies in vivo using electron microscopy identified the numerous bacteria occupying the apical cytoplasm of infected enterocytes which was otherwise composed almost entirely of free ribosomes and scattered mitochondria. These morphological findings associated with our molecular results suggest a global increase in the cell metabolism in response to L. intracellularis infection. Taking the host cell cycle events into consideration, an increase in protein synthesis and transcription is required during the G₁ phase in order to prepare the cell for subsequent division. Interestingly, this protein synthesis network was also associated with cell proliferation at an early stage of infection with human immunodeficiency virus.

The differential expression of cell cycle-associated genes identified by the functional clustering analysis (Table 8) showed specific activation of Rho family genes (RhoA, RhoB and Rho GTPase). These molecules play a key role in carcinogenesis through their aberrant activation which results in cell proliferation. Rho family proteins specifically act on the G₁-checkpoint of the cell cycle when the transition to commit the cell to the proliferative stage occurs. If the signals responsible for promoting this transition are not present then the cells enter into the non-proliferative phase (G₀). Additionally, a gene encoding Rho GTPase was also highly up-regulated during L. intracellularis infection in vitro, suggesting that exacerbated activation of the G₁ phase is an important mechanism involved in the proliferative changes induced by L. intracellularis in infected enterocytes (Oh Y S, Lee J B, McOrist S. Microarray analysis of differential expression of cell cycle and cell differentiation genes in cells infected with Lawsonia intracellularis. Vet J 2010; 184:340-5).

In addition to their roles in cancer development, Rho proteins are also pathologically activated by bacterial toxins also known as cyclomodulins. Cytotoxic necrotizing factor (CNF) found in uropathogenic Escherichia coli, Pasteurella multocida toxin (PMT) and dermonecrotic toxin of Bordetella spp. act directly on Rho family proteins to bring about their irreversible activation. Furthermore, CNF-expressing E. coli establish a persistent intracellular infection in the urogenital tract and suppress apoptosis by affecting the transcription levels of Bcl-2 family genes. It is thought that apoptosis inhibition in target cells may favor bacterial persistence at the epithelium surface, thereby favoring bacterial replication and spread inside the host cell. Although our study showed a predominant activation of anti-apoptotic-related genes compared with pro-apoptotic events, the Bcl-2 gene was not significantly activated in infected cells (log₂-fold change=1.06). Therefore, other mechanisms may be involved in the predominant activation of anti-apoptotic genes identified in the present study.

The contribution of apoptotic mechanisms for the pathogenesis of L. intracellularis infections has been speculated over the years and still needs to be elucidated. Initially, a temporary reduction in apoptosis was hypothesized to be an important mechanism involved in the cell proliferation. Later studies suggested an increase in apoptosis based on the Caspase-3 immunohistochemical staining. One of these studies described the dynamic of the Caspase-3 staining through the chronological evaluation of experimentally-infected animals and showed variations on the pattern of Caspase-3 staining within different parts of the intestinal mucosa on day 19 PI (Mulvey M A, Lopez-Boado Y S, Wilson C L, et al. Induction and evasion of host defenses by type 1-piliated uropathogenic Escherichia coli. Science 1998; 282:1494-7). Our study showed activation of the gene-encoding Caspase-3 (logₑ-fold change=1.12) in infected enterocytes, but the level did not reach the parameters for statistical significance. Considering the complexity and dynamism of the apoptosis process, the stage of the infection at the cellular level may directly influence the apoptotic gene network. Therefore, an in vitro model displaying the proliferative phenotype would be an ideal starting point to address this question.

In association with pro-apoptotic events, we identified down-regulation of two genes involved in the cellular immune response against intracellular pathogens (SLAM7 and TNFSF10) (Table 8). The apparent poor immune response in L. intracellularis infections has been indicated over the years. A microarray study also identified poor activation of immune response-related genes in general in field cases of PE. Despite the reduced expression of these two immune response-associated genes identified in our study, we observed significant up-regulation of MHC-I genes in infected cells, indicating that Lawsonia-derived antigen is presented to the lamina propria through the basal membrane of infected enterocytes (FIG. 10A).

Although the physiopathology of diarrhea in L. intracellularis infections remains to be elucidated, the significant down-regulation of numerous genes related to nutrient acquisition observed in the present study indicates that malabsorptive diarrhea represents the major mechanism involved in the poor performance and growth of affected animals. Supporting our observations, a lower absorption of glucose and electrolytes was reported using a hamster experimental model of PE. The reduced expression of nutrient acquisition-related genes also indicates the lack of cell differentiation in infected enterocytes.

The deficiency in nutrient acquisition described above seems to contrast with the increase in the cell metabolism characterized by the activation of transcription and protein synthesis-related genes also described here. As proposed in the FIG. 10A, the high expression of the glucose transporter SLC2A1 on the basolateral membrane appears to compensate for this lack of nutrient acquisition from the intestinal lumen, since it has been described as being expressed in the basolateral membrane of human enterocytes in vitro and rat jejunum in vivo.

The only gene significantly up-regulated in infected cells which is involved in nutrient acquisition and is physiologically expressed on the apical membrane is the high-affinity copper uptake gene (Sharp P A. Ctr1 and its role in body copper homeostasis. Int J Biochem Cell Biol 2003; 35:288-91). Copper is an essential metal used by eukaryotic cells as a biochemical cofactor especially during the process of oxygen reduction by cytochrome c oxidase which leads to the production of ATP. The production of ATP by the eukaryotic host is crucial for metabolism of Lawsonia and two other groups of obligate intracellular bacteria (Chlamydiales and Rickettsiales). These organisms express ATP/ADP translocase which catalyze the exchange of bacterial ADP for host ATP allowing bacteria to exploit their hosts' energy pool, a process referred to as energy parasitism. In addition to the ubiquitous essentiality for host cells, an increase in copper uptake has also been reported as a defense mechanism against intracellular pathogens because of its toxic properties. Free intracellular copper can lead to oxidative stress whereby cycling of copper oxidation and reduction produce reactive oxygen species through the Fenton reaction. Interestingly, our study identified L. intracellularis genes related to oxidative stress highly expressed within the host cytoplasm (FIG. 10B).

L. intracellularis genes belonging to the oxidative stress protection were the functional cluster most associated with genes highly expressed in the host cytoplasm. Although this fastidious organism requires a strict microaerophilic environment for cultivation in cell culture, Lawsonia is often located close to the cell mitochondria, where the transport of oxygen is continuous due to the oxidative phosphorylation. Additionally, this intracellular location is important to exploit the host energy pool by exchanging bacterial ADP for host generated ATP, as discussed earlier. Based on this scenario, our study demonstrated that L. intracellularis displays a sophisticated mechanism to survive in this microenvironment by coping with the oxidative stress and potentially incorporating intracytoplasmic copper taken by the host to combat the intracellular infection (FIG. 10A).

Our study identified high expression of an operon in which the first two genes (PcrH and SseC) were genetically related to the SPI2 and the last gene (hypothetical protein LI1159) was the third highest expressed gene by L. intracellularis. The proteins included within this operon act as a translocon attached to the phagosomal membrane to allow translocation of effector proteins into the cell cytoplasm. These effector proteins interfere with the intracellular trafficking favoring bacterial survival. Since we could not distinguish intracellular bacteria free in the cytoplasm from those organisms present within endosomes, we hypothesize that this operon was highly expressed by L. intracellularis organisms in the cell endosome (FIG. 10B).

We found five genes encoding hypothetical proteins among the ten highest expressed by L. intracellularis (Table 6). From the host transcriptome, we identified significant activation of Rho family genes which are crucial for the progression of the G₁ phase of the host cell cycle and are targeted by bacterial toxins. Taken together, this evidence suggests the presence of an unrecognized cyclomodulin encoded by those highly expressed genes of L. intracellularis.

### EXAMPLE 3

### Preparation and Quantification of L. intracellularis Inoculum

The porcine pathogenic L. intracellularis strain PHE/MN1-00 was previously isolated and continuously grown in cell culture using murine fibroblast-like McCoy cells (ATCC CRL 1696) for 10 passages (Guedes R M, Gebhart C J. Onset and duration of fecal shedding, cell-mediated and humoral immune responses in pigs after challenge with a pathogenic isolate or attenuated vaccine strain of Lawsonia intracellularis. Vet Microbiol 2003; 91:135-45). The bacteria were then pelleted, suspended in sucrose-potassium glutamate (pH 7.0; 0.218 M sucrose, 0.0038 M KH₂PO₄, 0.0072 M K₂HPO₄ and 0.0049 M potassium glutamate) solution with 10% fetal bovine serum and stored at −80° C. until the day of infection. The number of L. intracellularis organisms was assessed by direct counting after immunoperoxidase staining (Guedes R M, Gebhart C J, Deen J, et al. Validation of an immunoperoxidase monolayer assay as a serologic test for porcine proliferative enteropathy. J Vet Diagn Invest 2002; 14:528-30) and by quantitative PCR (qPCR), as described elsewhere (Vannucci F A, Pusterla N, Mapes S M, et al. Evidence of host adaptation in Lawsonia intracellularis infections. Vet Res 2012; 43:53).

### Tissue Collection and Preservation

All animals were euthanized 21 post-infection (PI) approaching the peak of the clinical disease, as described previously (Vannucci F A, Beckler D, Pusterla N, et al. Attenuation of virulence of Lawsonia intracellularis after in vitro passages and its effects on the experimental reproduction of porcine proliferative enteropathy. Vet Microbiol 2012). Immediately after euthanasia, the ileal mucosa was washed three times with PBS solution containing 2.0 U/μl RNAse inhibitor (Roche Applied Science) (Brown A L, Smith D W. Improved RNA preservation for immunolabeling and laser microdissection. RNA 2009; 15:2364-74.). A total of six tissue samples from each animal were collected, placed into plastic cryomold cassette (Tissue-Tek® Sakura Finetek) and embedded in optimal cutting temperature (OCT) compound (Tissue-Tek® Sakura Finetek). The samples were then placed onto blocks of dry ice until the tissues and OCT were frozen to a solid white. All samples were stored at −80° C. until use.

### Laser Capture Microdissection

The LCM procedures were performed in the second, third and fourth serially-cut slides using Histogene™ staining (Life Technologies). Briefly, the specimens were thawed at room temperature for 30 s, fixed in nuclease-free 75% ethanol, rehydrated in nuclease-free water for 30 s, stained with HistoGene® solution for 20 s and rinsed in water for 30 s, then dehydrated by sequential immersion into 75%, 95% and 100% ethanol for 30 s each. All microdissection procedures were performed at the College of Biological Sciences Imaging Center at the University of Minnesota using PixCell® II System (Life Technologies). Single-cells were captured from the slides using CapSure LCM caps (Life Technologies) with laser spot diameter 7.5 μm, power 60 mW and duration 650 μs. One thousand cells were captured from the bottom to the upper part of the intestinal crypts (FIGS. 7A and 7B) in each of the three stained slides in approximately ten minutes, incubated at 42° C. for 30 min in 500 extraction buffer and stored at −80° C. The microdissected cells from each of the three serially-cut slides were pooled, totaling 3000 cells, and used for the RNA extraction.

### RNA Isolation and Amplification

Total RNA was isolated from microdissected tissues using a PicoPure™ Kit (Life Technologies) according to the manufacturer's instructions. Genomic DNA was digested using DNase I (Qiagen). RNA samples were then assessed with a NanoDrop ND-8000 Spectrophotometer and Agilent Bioanalyzer 2100. Amplified cDNA was prepared from 100 ng of total RNA using the Ovation® RNA-Seq System V2 (Nugen®), following the manufacturer's instructions. The amplification was initiated at the 3′ end as well as randomly throughout the sample which allows the amplification of eukaryotic and prokaryotic RNA transcripts. The amplified cDNA generated was then purified using MinElute Reaction Cleanup Kit (Qiagen) for library preparation.

Along with the comprehensive host transcriptome, three to five percent of the sequence reads generated in our study also mapped against the L. intracellularis genome. We believe the reduced bias introduced during the RNA amplification step using random primers associated with the generation of amplified cDNA through SPIA (single primer isothermal amplification) technology was crucial in the enrichment of bacterial transcripts. However, the detection of prokaryote transcripts may vary among bacterial species especially due the wide variation in genomic composition and the amount of organisms in infected cells (Westermann A J, Gorski S A, Vogel J. Dual RNA-seq of pathogen and host. Nat Rev Microbiol 2012; 10:618-30).

### Library Preparation and Sequencing

The library preparation and sequencing were conducted in the core facility of the Biomedical Genomics Center at the University of Minnesota. Briefly, 100 ng of the amplified cDNA was fragmented, ends repaired, enriched by PCR and validated using High Sensitivity Chip on the Agilent2100 Bioanalyzer. Following quantification of the cDNA generated for the library using PicoGreen Assay, the samples were clustered and loaded on the Illumina® Genome Analyzer GA IIx platform which generated on average 22,136,064 paired reads of 100 bp. Base calling and quality filtering were performed following the manufacturer's instructions (Illumina® GA Pipeline).

### Filtering and Mapping of Sequence Reads

Quality control, trimming and mapping were performed in the Galaxy platform (Giardine B, Riemer C, Hardison R C, et al. Galaxy: a platform for interactive large-scale genome analysis. Genome Res 2005; 15:1451-5). Initially, FastQC tool was applied to the raw sequence data followed by FastQ Trimmer (Blankenberg D, Gordon A, Von Kuster G, et al. Manipulation of FASTQ data with Galaxy. Bioinformatics 2010; 26:1783-5). As a result, 15 base pairs were trimmed from the 5′end and ten from the 3′end of each raw sequence read. Sequences containing 75 bp and a phred quality score of more than 20 were used in the gene expression analysis. Using Bowtie short read aligner with no more than two mismatches, the filtered sequences were mapped onto the pig genome S. scrofa 10.2 and the L. intracellularis isolate PHE/MN1-00 reference genome, both obtained from the National Center for Biotechnology Information (NCBI) database (Langmead B, Trapnell C, Pop M, et al. Ultrafast and memory-efficient alignment of short DNA sequences to the human genome. Genome Biol 2009; 10:R25). The number of reads mapped within each annotated transcript was calculated in order to estimate the level of transcription for each gene. Cufflinks tool was used to estimate the relative abundances of the transcript reads for each gene (Trapnell C, Roberts A, Goff L, et al. Differential gene and transcript expression analysis of RNA-seq experiments with TopHat and Cufflinks. Nat Protoc 2012; 7:562-78). For comparing the levels of gene expression between infected and non-infected enterocytes and characterizing the abundance of L. intracellularis transcripts expressed in the host cytoplasm, the read counts were normalized based on the number of reads per kilobase of coding sequence per million mapped reads (RPKM).

In order to determine any potential co-infection associated with the intracytoplasmic L. intracellularis, the sequence reads were screened against 4,279 viral sequences available at NCBI database. Additionally, MetaPhlAn (Metagenomic Phylogenetic Analysis) was performed to identify other bacterial genome sequences in the experimental samples (Segata N, Waldron L, Ballarini A, et al. Metagenomic microbial community profiling using unique Glade-specific marker genes. Nat Methods 2012; 9:811-4).

### Differential Gene Expression

The differential gene expression between infected and non-infected enterocytes was assessed using the CuffDiff tool. This tool defines the total read count for each gene by combining the expression data from all the replicates in each experimental group and testing for differential regulation in expressed transcripts present in at least three replicates. RPKM values were expressed in log₂ (RPKM) to allow the statistical comparison. As a result, log₂-fold change in abundance of each transcript was obtained by log₂ (RPKM [infected]/RPKM [non-infected]). P-values were calculated and adjusted for multiple comparisons using false discovery rate (FDR) (Benjamini Y, Hochberg Y. Controlling the False Discovery Rate: A Practical and Powerful Approach to Multiple Testing. Journal of the Royal Statistical Society. Series B 1995; 57:289-300). Significant differential expression was determined in genes with FDR-adjusted p-values<0.05 and fold change±2.

### Functional Gene Ontology and Pathway Analysis

Biological functions and interactions of the genes differentially expressed were determined using the database for annotation visualization and integrated discovery (DAVID v6.7) and the Ingenuity pathway analysis (IPA) (Ingenuity® Systems). While the DAVID knowledge database was used for functional clustering, the mammalian knowledge database of the IPA was used for pathway and network analyses of the differentially expressed genes.

Predictive analysis regarding the ontology and motifs of gene-encoding hypothetical proteins highly expressed by L. intracellularis were performed using Kyoto Encyclopedia of Genes and Genomes (KEGG) database. Additionally, Phyre² (Protein Homology/analogY Recognition Engine) (Kelley L A, Sternberg M J. Protein structure prediction on the Web: a case study using the Phyre server. Nat Protoc 2009; 4:363-71) and ConFunc (Protein Function Prediction Server) (Wass M N, Sternberg M J. ConFunc—functional annotation in the twilight zone. Bioinformatics 2008; 24:798-806) software were used to predict their biological functions and three-dimensional structure.

### Reverse Transcription PCR

In addition to the assessment of RNA quality based on the bacterial and eukaryotic ribosomal RNAs using Agilent Bioanalyzer 2100, one-step RT-PCR was applied to the RNA extracted from microdissection tissues in order to evaluate the quality of bacterial mRNA for RNA-seq analysis. The one-step RT-PCR (Qiagen®) was used with specific primers (Table 9) targeting three housekeeping genes of L. intracellularis.

Quantitative RT-PCR (qRT-PCR) was performed for validating the expression data identified by RNA-seq using a specific set of genes from the host and bacterium (Table 9). RNA samples were synthesized to first-strand cDNA using SuperScript® II RT (Invitrogen). Duplicate qRT-PCR reactions from each primer probe set were validated by five serial dilutions of cDNA on the ABI7900HT instrument (Applied Biosystems). After validation, quantitative PCR was performed in duplicate using 15 ng of cDNA per sample with the following conditions: 60° C. for 2 min, 95° C. for 5 min and 45 cycles (95° C./10 sec and 60° for 1 min). The differential expression for the host transcriptome were described as relative fold-change between infected and non-infected enterocytes based on Ct values (2^{-ΔΔCt}), as previously described (Bookout A L, Cummins C L, Mangelsdorf D J, et al. High-throughput real-time quantitative reverse transcription PCR. Curr Protoc Mol Biol 2006; Chapter 15: Unit 15.8). The suitability of four porcine housekeeping genes (Beta actin, Cyclophilin A, Hypoxanthine phosphoribosyltransferase, Ribosomal protein L4) for qRT-PCR analysis was evaluated.

Based on consistent Cyclophilin A gene expression throughout the biological replicates, this gene was used to normalize the expression data. For the bacterial transcriptome, averages of relative transcriptional levels normalized by the 30S ribosomal gene were calculated and compared to the RNA-seq expression levels. A linear regression model was used to evaluate the correlation between average RPKM and qRT-PCR data, as described previously (Vannucci F A, Foster D N, Gebhart C J. Comparative Transcriptional Analysis of Homologous Pathogenic and Non-Pathogenic Lawsonia intracellularis Isolates in Infected Porcine Cells. PLoS One 2012; 7:e4670).

### Results

Technical Characterization of the LCM Coupled with RNA-seq Analysis

Based on previous studies reporting the chronological course of the L. intracellularis infection in vivo, experimentally-infected pigs in our study were monitored every other day regarding clinical signs (diarrhea and general attitude) and quantitative fecal shedding of L. intracellularis DNA until the end point of the study (21 PI). The negative control group was also monitored and its negative status was consistent throughout the study period. One animal from the infected group exhibiting only mild to moderate lesions typical of PE associated with grade 1 and 2 in the IHC grading scheme was discarded from the experiment. In order to have the same number of replicates in both infected and control groups, one animal was randomly discarded from the negative control group. As a result, five biological replicates (animals) from each group were included in the LCM procedures and RNA-seq analysis.

In order to screen for the presence of any other microbial organisms in the experimental samples, the sequence reads were also mapped against viral, bacterial and archaeal genome databases. While no detectable hits were found against the viral and archaeal genomes, 0.052% and 0.084% of the sequence reads from the infected and control group, respectively, were mapped against the bacterial database. Three unculturable bacterial species (Candidatus Zinderia, Candidatus Carsonella ruddii, Candidatus Sulcia muelleri) and Propionibacterium acnes were commonly found in both groups. An uncharacterized species of the genus Peptoniphilus previously reported in the oral cavity of a human was exclusively found in the control group.

Less than 0.1% of the sequence reads mapped against four bacterial species commonly found in both infected and non-infected groups, when screened for the presence of virus and other bacteria. While three of these species represent unculturable organisms (Candidatus) related to insect symbionts, one (Propionibacterium acnes) is part of the normal flora of the skin, oral cavity, large intestine, the conjunctiva and the external ear canal of humans. The identification of the genus Peptoniphilus, found exclusively in the control group, may be due to human contamination during the experimental procedures, since it was reported in the oral cavity through the human microbiome project (NCBI accession: PRJNA52051).

Although the foregoing specification and examples fully disclose and enable the present invention, they are not intended to limit the scope of the invention, which is defined by the claims appended hereto. All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for detecting the presence of a Lawsonia intracellularis biomarker in an animal, comprising identifying in a physiological sample from the animal (i) an antibody specific for an amino acid of SEQ ID NO:2 (LI0447), LI0461, LI0267, LI0809, LI1158, LI1159 or LIC060, or (ii) an amino acid of SEQ ID NO:2, LI0461, LI0267, LI0809, LI1158, LI1159 or LIC060.

2. The method of claim 1, wherein the sample comprises an antibody.

3. The method of claim 1, wherein the biomarker is an amino acid of SEQ ID NO:2, LI0461, LI0267, LI0809, LI1158, LI1159 or LIC060.

4. The method of claim 1, wherein the animal is a pig or a horse.

5. An immunogenic composition comprising a purified polypeptide encoded by a nucleic acid sequence having at least 90% identity to a nucleic acid sequence SEQ ID NO:1, or a nucleic acid having at least 90% identity to a nucleic acid sequence encoding LI0461, LI0267, LI0809, LI1158, LI1159 or LIC060.

6. The immunogenic composition of claim 5, wherein the nucleic acid sequence consists of nucleic acid sequence SEQ ID NO:1, or a nucleic acid sequence encoding LI0461, LI0267, LI0809, LI1158, LI1159 or LIC060.

7. The immunogenic composition of claim 5, which further comprises a physiologically-acceptable vehicle.

8. The immunogenic composition of claim 5, which further comprises an effective amount of an adjuvant.

9. The immunogenic composition of claim 5, wherein the polypeptide is capable of generating an antibody that specifically recognizes the polypeptide, and wherein the amount of the immunogenic composition is effective to ameliorate colonization or infection by L. intracellularis in a susceptible mammal.

10. A method of treating a susceptible animal against colonization or infection by L. intracellularis comprising administering to the animal an effective amount of the immunogenic composition of claim 5, wherein the polypeptide is capable of generating an antibody specific to the polypeptide, and wherein the amount of the immunogenic composition is effective to ameliorate colonization or infection by L. intracellularis in the susceptible animal.

11. The method of claim 10, wherein the immunogenic composition is administered by subcutaneous injection, by intramuscular injection, by oral ingestion, intranasally, or combinations thereof.

12. The method of claim 10, wherein the animal is a pig or a horse.

13. A vaccine comprising an immunogenic amount of the composition of claim 5, which amount is effective to inhibit in an animal an infection by L. intracellularis, in combination with a physiologically-acceptable, non-toxic vehicle.

14. The composition of claim 5, wherein the polypeptide has the amino acid sequence of SEQ ID NO:2, LI0461, LI0267, LI0809, LI1158, LI1159 or LIC060.

15. A purified antibody that binds specifically to the amino acid of SEQ ID NO:2, LI0461, LI0267, LI0809, LI1158, LI1159 or LIC060.


SEQUENCE LISTING (86 sequences)

SEQ ID NO: 1 - DNA, 294 bp, Lawsonia intracellularis
atgggcgactgtcattcatctaggaatatagttacctatattctcaagcaacctacccaggaacattggccgggtcagcctgttcccctatttggtcttgcttcaaacggggcttgcctagctcgctatatcactataacgactggtagactcttactccaccgtttcacccttaccactattattaataatggcggtttactttctgttgcgcttgccgaagatcactcttcctgggtgttacccagcgttttaccctatgaagcccggactttcctctccataaatccataa

SEQ ID NO: 2 - protein, 97 aa, Lawsonia intracellularis
MGDCHSSRNIVTYILKQPTQEHWPGQPVPLFGLASNGACLARYITITTGRLLLHRFTLTTIINNGGLLSVALAEDHSSWVLPSVLPYEARTFLSINP

SEQ ID NO: 3 - DNA, 22 bp, Artificial Sequence
  Description of Artificial Sequence Synthetic primer
tcatatccatgaaggaggatca

SEQ ID NO: 4 - DNA, 22 bp, Artificial Sequence
  Description of Artificial Sequence Synthetic primer
ggagtccagctgttagatgtcc

SEQ ID NO: 5 - DNA, 25 bp, Artificial Sequence
  Description of Artificial Sequence Synthetic primer
caagcaactgtttaccgtactatga

SEQ ID NO: 6 - DNA, 25 bp, Artificial Sequence
  Description of Artificial Sequence Synthetic primer
tcactaaaacgtacttctcgtgcta

SEQ ID NO: 7 - DNA, 20 bp, Artificial Sequence
  Description of Artificial Sequence Synthetic primer
cccagcgttttaccctatga

SEQ ID NO: 8 - DNA, 21 bp, Artificial Sequence
  Description of Artificial Sequence Synthetic primer
tcgctgcatatccttttatgg

SEQ ID NO: 9 - DNA, 23 bp, Artificial Sequence
  Description of Artificial Sequence Synthetic primer
tcataacagtttttgcaacatgg

SEQ ID NO: 10 - DNA, 20 bp, Artificial Sequence
  Description of Artificial Sequence Synthetic primer
gcaataatcccaggcatttc

SEQ ID NO: 11 - DNA, 24 bp, Artificial Sequence
  Description of Artificial Sequence Synthetic primer
tgcatgaaacagtcattaatcaaa

SEQ ID NO: 12 - DNA, 27 bp, Artificial Sequence
  Description of Artificial Sequence Synthetic primer
ccctaaattattatgaccatcagtgag

SEQ ID NO: 13 - DNA, 27 bp, Artificial Sequence
  Description of Artificial Sequence Synthetic primer
aagatcaaaaattattaccagtttgct

SEQ ID NO: 14 - DNA, 21 bp, Artificial Sequence
  Description of Artificial Sequence Synthetic primer
gccataatgcaaccatgtacc

SEQ ID NO: 15 - DNA, 20 bp, Artificial Sequence
  Description of Artificial Sequence Synthetic primer
gggaaatccttgcccttatc

SEQ ID NO: 16 - DNA, 23 bp, Artificial Sequence
  Description of Artificial Sequence Synthetic primer
aaaacacctgcgacagtacctaa

SEQ ID NO: 17 - DNA, 20 bp, Artificial Sequence
  Description of Artificial Sequence Synthetic primer
aaagcaccacagcattagca

SEQ ID NO: 18 - DNA, 24 bp, Artificial Sequence
  Description of Artificial Sequence Synthetic primer
catctaggtcaatgaaaagtacgc

SEQ ID NO: 19 - DNA, 21 bp, Artificial Sequence
  Description of Artificial Sequence Synthetic primer
catctgagtggttagctgcaa

SEQ ID NO: 20 - DNA, 24 bp, Artificial Sequence
  Description of Artificial Sequence Synthetic primer
cacaaaagctaaaacaatccctct

SEQ ID NO: 21 - DNA, 22 bp, Artificial Sequence
  Description of Artificial Sequence Synthetic primer
cagcaagaaatacaggtgcaag

SEQ ID NO: 22 - DNA, 21 bp, Artificial Sequence
  Description of Artificial Sequence Synthetic primer
ttgctcacgggactttttatc

SEQ ID NO: 23 - DNA, 18 bp, Artificial Sequence
  Description of Artificial Sequence Synthetic primer
ggcagaagatggggatga

SEQ ID NO: 24 - DNA, 20 bp, Artificial Sequence
  Description of Artificial Sequence Synthetic primer
gctcccgccttactcttctt

SEQ ID NO: 25 - DNA, 20 bp, Artificial Sequence
  Description of Artificial Sequence Synthetic primer
cggcacagggtactttctct

SEQ ID NO: 26 - DNA, 20 bp, Artificial Sequence
  Description of Artificial Sequence Synthetic primer
atgctctgtgatgtccacga

SEQ ID NO: 27 - DNA, 20 bp, Artificial Sequence
  Description of Artificial Sequence Synthetic primer
gatttcccctggacaagagc

SEQ ID NO: 28 - DNA, 22 bp, Artificial Sequence
  Description of Artificial Sequence Synthetic primer
ctgaagaaaatccatgtgtcca

SEQ ID NO: 29 - DNA, 20 bp, Artificial Sequence
  Description of Artificial Sequence Synthetic primer
attagcgtgggctgcatagt

SEQ ID NO: 30 - DNA, 22 bp, Artificial Sequence
  Description of Artificial Sequence Synthetic primer
gatagagaaggctggaatgagg

SEQ ID NO: 31 - DNA, 18 bp, Artificial Sequence
  Description of Artificial Sequence Synthetic primer
tcagttgttcccgcgtct

SEQ ID NO: 32 - DNA, 18 bp, Artificial Sequence
  Description of Artificial Sequence Synthetic primer
cacctgctccaccagtcc

SEQ ID NO: 33 - DNA, 20 bp, Artificial Sequence
  Description of Artificial Sequence Synthetic primer
ggatatgggatccgacacag

SEQ ID NO: 34 - DNA, 18 bp, Artificial Sequence
  Description of Artificial Sequence Synthetic primer
ggtggaggctggtagctg

SEQ ID NO: 35 - DNA, 18 bp, Artificial Sequence
  Description of Artificial Sequence Synthetic primer
atggcggtgatgcagact

SEQ ID NO: 36 - DNA, 22 bp, Artificial Sequence
  Description of Artificial Sequence Synthetic primer
gatcaggatcaacacacaggtc

SEQ ID NO: 37 - DNA, 20 bp, Artificial Sequence
  Description of Artificial Sequence Synthetic primer
tgttggctggcaagacatag

SEQ ID NO: 38 - DNA, 20 bp, Artificial Sequence
  Description of Artificial Sequence Synthetic primer
gcgtaaatcacccacaatcc

SEQ ID NO: 39 - DNA, 20 bp, Artificial Sequence
  Description of Artificial Sequence Synthetic primer
tccagctgatcggaatcttt

SEQ ID NO: 40 - DNA, 22 bp, Artificial Sequence
  Description of Artificial Sequence Synthetic primer
gaggaataggcccatgttacac

SEQ ID NO: 41 - DNA, 18 bp, Artificial Sequence
  Description of Artificial Sequence Synthetic primer
tggctctgggaatgatgc

SEQ ID NO: 42 - DNA, 21 bp, Artificial Sequence
  Description of Artificial Sequence Synthetic primer
tccagagttgacccacagttt

SEQ ID NO: 43 - DNA, 20 bp, Artificial Sequence
  Description of Artificial Sequence Synthetic primer
tgcgtcatgtgtgggtactt

SEQ ID NO: 44 - DNA, 21 bp, Artificial Sequence
  Description of Artificial Sequence Synthetic primer
catccctggcatcactatgac

SEQ ID NO: 45 - DNA, 19 bp, Artificial Sequence
  Description of Artificial Sequence Synthetic primer
aagttccgcagatccgaag

SEQ ID NO: 46 - DNA, 20 bp, Artificial Sequence
  Description of Artificial Sequence Synthetic primer
ccacatgtgacgtgaggtct

SEQ ID NO: 47 - DNA, 20 bp, Artificial Sequence
  Description of Artificial Sequence Synthetic primer
tggagcctgaagatttgtcc

SEQ ID NO: 48 - DNA, 20 bp, Artificial Sequence
  Description of Artificial Sequence Synthetic primer
ccgggtttgagttagctgat

SEQ ID NO: 49 - DNA, 20 bp, Artificial Sequence
  Description of Artificial Sequence Synthetic primer
agctggcccatcagtctaac

SEQ ID NO: 50 - DNA, 19 bp, Artificial Sequence
  Description of Artificial Sequence Synthetic primer
atcatcatcaccggggttc

SEQ ID NO: 51 - DNA, 22 bp, Artificial Sequence
  Description of Artificial Sequence Synthetic primer
ggaattgttatttctcggtcca

SEQ ID NO: 52 - DNA, 19 bp, Artificial Sequence
  Description of Artificial Sequence Synthetic primer
tgtctccaagccagtgtcc

SEQ ID NO: 53 - DNA, 19 bp, Artificial Sequence
  Description of Artificial Sequence Synthetic primer
agcagcccaaggatctctc

SEQ ID NO: 54 - DNA, 23 bp, Artificial Sequence
  Description of Artificial Sequence Synthetic primer
tgctgagatctgtcagtttgagt

SEQ ID NO: 55 - DNA, 22 bp, Artificial Sequence
  Description of Artificial Sequence Synthetic primer
tcatatccatgaaggaggatca

SEQ ID NO: 56 - DNA, 22 bp, Artificial Sequence
  Description of Artificial Sequence Synthetic primer
ggagtccagctgttagatgtcc

SEQ ID NO: 57 - DNA, 20 bp, Artificial Sequence
  Description of Artificial Sequence Synthetic primer
cccagcgttttaccctatga

SEQ ID NO: 58 - DNA, 21 bp, Artificial Sequence
  Description of Artificial Sequence Synthetic primer
tcgctgcatatccttttatgg

SEQ ID NO: 59 - DNA, 22 bp, Artificial Sequence
  Description of Artificial Sequence Synthetic primer
cagcttgtaatgactccccagt

SEQ ID NO: 60 - DNA, 20 bp, Artificial Sequence
  Description of Artificial Sequence Synthetic primer
tcagctaattcaacccgtga

SEQ ID NO: 61 - DNA, 20 bp, Artificial Sequence
  Description of Artificial Sequence Synthetic primer
agctgcggttgaagaaggta

SEQ ID NO: 62 - DNA, 20 bp, Artificial Sequence
  Description of Artificial Sequence Synthetic primer
ttaatggagcggacaaaagc

SEQ ID NO: 63 - DNA, 25 bp, Artificial Sequence
  Description of Artificial Sequence Synthetic primer
gtagaacaatctttagcaacggagt

SEQ ID NO: 64 - DNA, 27 bp, Artificial Sequence
  Description of Artificial Sequence Synthetic primer
aataatcccatcagttatagtttctgc

SEQ ID NO: 65 - DNA, 21 bp, Artificial Sequence
  Description of Artificial Sequence Synthetic primer
catctgagtggttagctgcaa

SEQ ID NO: 66 - DNA, 24 bp, Artificial Sequence
  Description of Artificial Sequence Synthetic primer
cacaaaagctaaaacaatccctct

SEQ ID NO: 67 - DNA, 24 bp, Artificial Sequence
  Description of Artificial Sequence Synthetic primer
tgcatgaaacagtcattaatcaaa

SEQ ID NO: 68 - DNA, 27 bp, Artificial Sequence
  Description of Artificial Sequence Synthetic primer
ccctaaattattatgaccatcagtgag

SEQ ID NO: 69 - DNA, 22 bp, Artificial Sequence
  Description of Artificial Sequence Synthetic primer
cagcaagaaatacaggtgcaag

SEQ ID NO: 70 - DNA, 21 bp, Artificial Sequence
  Description of Artificial Sequence Synthetic primer
ttgctcacgggactttttatc

SEQ ID NO: 71 - DNA, 20 bp, Artificial Sequence
  Description of Artificial Sequence Synthetic primer
gggaaatccttgcccttatc

SEQ ID NO: 72 - DNA, 23 bp, Artificial Sequence
  Description of Artificial Sequence Synthetic primer
aaaacacctgcgacagtacctaa

SEQ ID NO: 73 - DNA, 23 bp, Artificial Sequence
  Description of Artificial Sequence Synthetic primer
aggatcttcaggaagttcgtctt

SEQ ID NO: 74 - DNA, 20 bp, Artificial Sequence
  Description of Artificial Sequence Synthetic primer
cattgcctggagtaccaaca

SEQ ID NO: 75 - DNA, 20 bp, Artificial Sequence
  Description of Artificial Sequence Synthetic primer
ttcttccatcccccataaca

SEQ ID NO: 76 - DNA, 20 bp, Artificial Sequence
  Description of Artificial Sequence Synthetic primer
ttggagacatccctttcctg

SEQ ID NO: 77 - DNA, 20 bp, Artificial Sequence
  Description of Artificial Sequence Synthetic primer
tttgctgcaataacgtgcat

SEQ ID NO: 78 - DNA, 20 bp, Artificial Sequence
  Description of Artificial Sequence Synthetic primer
gcacatattgctgggcttgt

SEQ ID NO: 79 - DNA, 20 bp, Artificial Sequence
  Description of Artificial Sequence Synthetic primer
ccataagctcaagagcacga

SEQ ID NO: 80 - DNA, 20 bp, Artificial Sequence
  Description of Artificial Sequence Synthetic primer
ctgcagcacttgcaaacaat

SEQ ID NO: 81 - protein, 498 aa, Lawsonia intracellularis
MKKVLISAFLFVGTLLTASLSAEAIDFKAKGVWDFNFEWQQSSFMKGDGDQRFSPKQRLRTQIDIVASESLKGVVFFELGKTIWGRGVDGASIGTDGKNVIKLRYSYVDWVIPYTDVQVRMGLQPYVLPGFVAGSTILDADGAGVTVSAVFNDYLGATAFWMRALHKNYDSNYGISKLPNFKGTTLDVVGLTIPVTISDIKIAPWGMFAFAGKKSLLGESYGDIEDVRAGLLPAMPAGFGYSWGAGNPFGDVFPNKKRGNAWWVGLSAELAGSSPLHIAVDGAYGRADLGSLRNVVIGDFLLDKIDLKRQGWYAALLAEYKFEYVTPGVIGWYASGDKVDSRGASKRIPTLVGNWSATSFGYSGAYGIGKDSVFGNTIAGSWGVVVQLKDISFLENLTHVIRGARIQGTNNKDVPEHLGLSYVTTIYDTRGGDNMLYLTKKDYAWEVDFDTEYKIYKDLSVALELSYIRLELDKKLWNLQREVDKNAYRAGLNMKYQF

SEQ ID NO: 82 - protein, 122 aa, Lawsonia intracellularis
MKIYALMICSLFFALCLLTTPVAIANNVQCDLTGRDWEKSSTNEKLSFLYGVSSVVVIEQEIAHKEGRKPSLFVQKWIKTFKDDTWMNIQHKIDKWYKEHPTQKNKDVLWVLWYEFMAPNTK

SEQ ID NO: 83 - protein, 129 aa, Lawsonia intracellularis
MKRLVIVLSIVCVLSAAESVFALNKKLDIMNGSQMDIYTLKVSPTNANQWEENLLQEETLKNGDKAMVEISRTENAEAWDITVTDKNGNESSWIGLPLDKAGDVTLLPDGQYLTDKDMTIDNMKKVMKK

SEQ ID NO: 84 - protein, 396 aa, Lawsonia intracellularis
MTNFGDISGSSARMSSLMTGTSGEEGLEELEGGVPKEQGGPGKGDASEAAKGQAAADSINSAGGTEKPGEVGDKEDVGEGGEIPEGGEIPEGGEEVPEEPPYVPPPLVEPAKISTVTDLSTLMGSLQLTEQKKNAEKTVNEIKAQNKEQQVKFQEQIKKIEDNIAESKKSGILKFFQKLFAVIGAVLGAIGGALAIAAGAASGNPLLVAAGIMAIVASIDAAMSSLSDGKVSISAGISKALEAMGVPAETAQWIAFGIQLAMIAVTIAIGFASGGGGAMAGVSKIADMFSKSQDVAKLAQMIEKASKIVQIAGSVNQSAIGGTGIGTAVVQSNIKANESEQKEIEAAIAKVKAKIETLQDFFKNQMEQFNAIMKIITDIIQDSVNTKIAVQRGARE

SEQ ID NO: 85 - protein, 265 aa, Lawsonia intracellularis
MLEKTIMISLNNSSIQIPQQNIEESTSQEVTSSSGGQPAKVDGVSIQAPKAPVTSAASNLEGVQQREAQENVTKMGLPELSAPKGGGYVQSTAAMFAEVTVDAMNQQRKAAQDVQNSALEGMVNKMLEAAKDIKEQSKLMMGLGIASSVMTTAMGFGGTIGGVKSMTANPGVGTTPQAQVTSNKLTLGKEIAEGIRGGMDTGKESQVKSIDADIRTTQAEEKKLEAQMELVKQFAENSKNLAQQALQIMSEVTRDANATTQRILA

SEQ ID NO: 86 - protein, 619 aa, Lawsonia intracellularis
MKNYSLTMWNIYKIIYIYTVVISKLYFYFKSNYQYGKRLFLYFPIQLLSTVKRFLIVATLVCFCILFYSNESFSMNGSEEERQPLLSSTSTTSSSSTSSSSSGANGYYSYPSYSQPQSTYPEVSAAEAGTACGCGSGAIGYSMLAVMNPSMSSLCGPAGWVLSSVGGACVGAGVSLACYLCGYNRWMGWEDHSTSNSIRTGGEGSTTMYGSTGTHQHREPESPDEGPSVSFPPVTVHQPASPGSPVVSQPGGRGRSSSYLTAEASLLGQMSSLHSMQCLLSNGSEQVLTTLRMLLLKNYSDSSKSQDFSFFKKNYINKDKGKVVSVKRKRGSLSTTIDVPSFLPYRFFSSIKPQDIHKRKVCSGQIGVLLDFSPGLTCGFAYDSNKKESKEYKGIQLGSTIGSVKSKMETEGLSAIVALNPANAGVTGHLVSSYNWGKVKNTRKIIHKGQEIITKGSPKVNLLGGLVQLGYNFFLLNQVTLVPYVECMFSSVSWSSYKEQYSPGACKFGKNKEQILEKSIGLRSDIGVSMNSSMQLWVAGISGYSKTNSLISSSYLSSYYRHNLSTYAKKYQYIRTELGVSYDGTFSDNVSVSLSGLFRLNKWSKVDNQNIVAQVQYTY